(19) United States
(12) Patent Application Publication
(10) Pub. No.: US 2007/0178572 A1
(43) Pub. Date: Aug. 2, 2007

(54) Protein crystal structure

(76) Inventors: Steven John Gamblin, London (GB); Jonathan Robert Wilson, London (GB); Philip Auld Walker, London (GB); Chun Jing, London (GB); George Michael Blackburn, Sheffield (GB); Bing Xiao, London (GB)

Correspondence Address: MORGAN LEWIS & BOCKIUS LLP, 1111 PENNSYLVANIA AVENUE NW, WASHINGTON, DC, 20004, US

(21) Appl. No.: 10/542,808

(22) PCT Filed: Jan. 22, 2004
(86) PCT No.: PCT/GB04/00233

§ 371 Date: Apr. 25, 2006

(30) Foreign Application Priority Data
Jan. 22, 2003 (GB) 0301450.3

(51) Int. Cl. C12N 9/12; G06F 19/00

(52) U.S. Cl. 435/194; 702/19

(57) ABSTRACT

Disclosed is a crystal comprising at least a catalytically active portion of a SET 7/9 histone methyltransferase, and various uses thereof, and ligands for SET 7/9.

### FIELD OF THE INVENTION

This invention relates to a protein crystal structure, various methods of utilising the structure and/or information derivable therefrom, and altered proteins.

### BACKGROUND OF THE INVENTION

In eukaryotic cells, DNA is maintained in a highly ordered and condensed in association with small, basic histone proteins. This packaged DNA is termed chromatin. Generally speaking, there are two forms of chromatin: heterochromatin which is tightly compacted and highly refractory to processes such as gene transcription; and euchromatin, which has a more open conformation and tends to be amenable to transcription.

The basic unit of chromatin is the nucleosome, which consists of approximately two turns of DNA around a histone core octamer comprising two monomers each of histones H2A, H2B, H3 and H4. The N terminal tails of the core histones protrude out of the core structure and make contact with adjacent nucleosomes.

The basic N terminal tails of the core histones are known to be subject to many different covalent modifications including acetylation and, in particular, methylation of lysine residues.

It is generally believed that modification of the histone tails affects chromatin structure and thereby has an impact on expression of genes within the DNA in the modified nucleosome. Thus it is widely held that histone modification provides a layer of epigenetic control of gene expression, although the details of the mechanisms involved have not yet been fully elucidated.

A number of enzymes which methylate histones (histone methyltransferases, or HMTs) are known. They tend to be highly specific e.g. a particular HMT will normally methylate only a particular lysine residue of a particular histone molecule (say, for example, the lysine residue at position 9 of histone H3). HMTs almost exclusively belong to the “SET” family of proteins, that is they contain a conserved methyltransferase domain called a SET domain (so called because the domain was first identified in the Drosophila protein Suvar)3-9, (Enhancer-of-zeste, Ttrithorax); (Su(var) is an abbreviation for “suppressor of variegation”).

It has been postulated that deregulation of SET domain function in SET proteins may be involved in the causation of many types of cancer (Schneider et al, 2002 TRENDS in Biochemical Sciences 27, 396-402) and, given the likely role of HMTs in regulation of gene expression, one can appreciate the basis for such a postulation.

The first example of an enzyme that specifically methylates lysine-4 of histone H3 in humans was independently characterised by two groups and separately named SET7 (Wang et al, 2001 Mol. Cell. 8, 1207-1217) and SET9 (Nishioka et al, 2002 Genes Dev. 16, 479-489). Accordingly the protein is now known as SET 719). This protein is among the smallest shown to possess HMTase activity and it also lacks SET domain-associated cysteine-rich regions.

The results of X-ray crystallographic analysis of the enzyme in isolation, or in complex with a reaction product, have been published (Wilson et al, 2002 Cell 777, 105-115; Jacobs et al, 2002 Nat. Struct. Biol. 9, 833-838). However; the crystal used for analysis by Wilson et al lacked a C-terminal segment of the enzyme (which is critical for catalytic activity) since no useful crystals could be obtained of the complete enzyme, due to the flexibility of the C terminal.

The SET 7/9 HMT comprises a conserved SET domain (i.e. the SET 7/9 domain) and flanking pre- and post-SET domains. Analysis by Rea et al, (2000 Nature 406, 593-599) suggested that, at least for SuV39H1, both the pre-SET and post-SET domains were required for HMTase activity. However, the function of the pre-SET and post-SET domains is still unclear. A variety of different sequences are found adjacent to the C terminus of SET domains across the family of HMTs, and this might reflect important differences in the precise specificity of the various enzymes.

The present inventors have now been able to prepare a crystal of a catalytically competent portion of the enzyme, in a complex with both its co-factor and a lysine-containing peptide substrate. This complex has allowed determination of the structure of the SET domain, including the essential C-terminal segment, to a surprisingly high resolution.

This information has, in turn, led the inventors to make a number of other inventions, described and exemplified below.

### SUMMARY OF THE INVENTION

In a first aspect the invention provides a crystal comprising at least a catalytically active portion of a SET 7/9 histone methyltransferase (HMT). In a preferred embodiment the crystal comprises SET 7/9 HMT in complex with a lysine-containing protein or peptide substrate and/or a methyl group-donating co-factor. It should be noted that the crystal complex may be formed before or (more preferably) after the enzyme-catalysed methylation reaction takes place, so the co-factor, if present, may be methylated or demethylated and, conversely, the substrate (if present) may be unmethylated or methylated.

In a preferred embodiment the invention provides a crystal comprising SET 7/9 HMT, optionally in a complex with a lysine-containing substrate and/or a (methyl group-donating) co-factor, which diffracts X-rays so as to allow for the determination of atomic co-ordinates of SET 7/9 HMT to a resolution of 1.7 Å or better.

A crystal in accordance with the invention typically has the space group P2₁, and unit cell dimensions of a=52.7 Å, b=75.4 Å, c=69.1 Å and β=94.2°.

In particular, a crystal in accordance with the invention comprises at least residues 117-366 of SET 7/9 HMT, which portion is catalytically active.

One crystal in accordance with the invention has the relative atomic co-ordinates set out in Annex 1, and these will generally be applicable for other crystals in accordance with the invention. However, those skilled in the art will appreciate that minor deviation from these precise co-ordinates will not significantly affect the structure, either in terms of its dominant characteristics or its usefulness in, for example, rational drug design (as discussed further below). Accordingly, for the purposes of the present specification, a crystal having a structure in which the atomic co-ordinates set out in Annex 1 may vary by up to 0.2 Å (more preferably no more than 0.1 Å) in any direction is considered as being a crystal in accordance with the present invention.

By way of explanation, Annex 1 shows (from left to right): the atom number and type, the residue type and number, the x, y and z co-ordinates of the atom (in Å); “OCC” is the occupancy and B is the B factor (in Å²). Atoms 1-3813 belong to the SET 7/9 domain. Atoms 3814-3855 belong to the S-AdoHomocysteine cofactor. Atoms 3859-4039 belong to the substrate peptide.

Using the data provided by the atomic co-ordinates, the inventors have identified residues of the SET 7/9 domain which are believed to interact with residues of the substrate. Thus in one particular embodiment the invention provides a crystal comprising a complex of SET 7/9 with a lysine-containing substrate peptide comprising at least the amino acid sequence ARTKQT, and wherein the complex involves one or more (preferably two or more, more preferably three or more, and most preferably four or more) of the interactions set out in Table 1 below. (The relative substrate residue numbering makes the lysine residue to be methylated position 0, with residues to the N terminal side being negative and residues to the C terminal side being positive):

TABLE 1

| SET 7/9 | |
|---|---|
| Residue interacts with: | Relative Substrate Residue |
| Trp 260 | −3 (e.g. Ala) |
| His 252 | −2 (e.g. Arg) |
| Asp 256 | −2 (e.g. Arg) |
| Ser 268 | −1 (e.g. Thr) |
| Thr 266 | Lys 0 |
| Ser 268 | Lys 0 |
| Ser 268 | +1 (e.g. Gln) |
| Val 355 | +1 (e.g. Gln) |
| Lys 317 | +2 (e.g. Thr) |

Those skilled in the art will readily appreciate that determination of the three dimensional ternary structure of SET 7/9 HMT in complex with its cofactor and/or a substrate provides the basis for the rational design of molecules which interact specifically with SET 7/9 HMT (alone, or in complex with its cofactor and/or a substrate). Such molecules may be referred to, for present purposes, as ligands. Of particular interest are ligands which will modulate the methyltransferase activity of SET 7/9 HMT. Such modulation may include inhibiting or enhancing the methylstransferase activity, altering its substrate specificity, and stabilising or destabilising the interaction between SET 7/9 HMT and its cofactor and/or a histone substrate. For present purposes, “destabilising” encompasses inhibition of the formation of a complex of SET 7/9 HMT with a substrate and/or its cofactor.

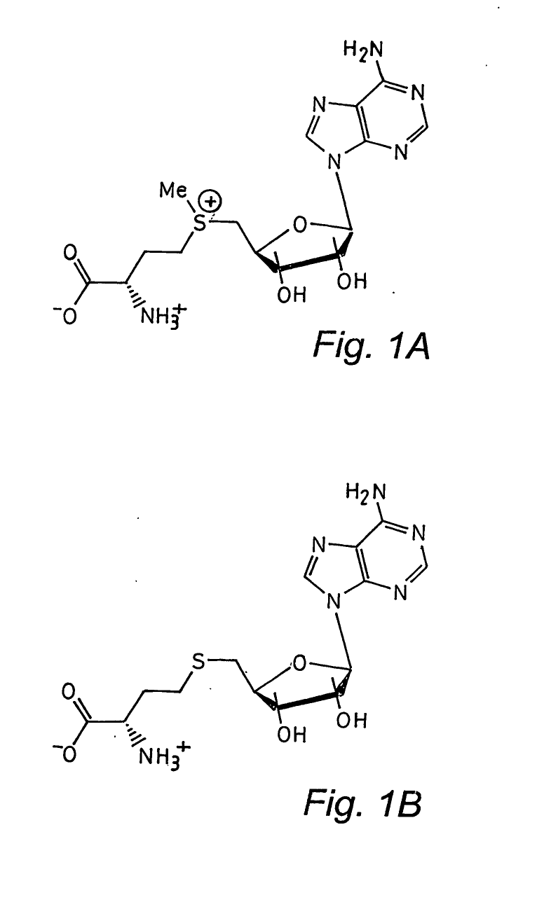

By way of explanation, SET 7/9 HMT requires a source of methyl groups for transfer to the lysine residue of the substrate to be methylated. The source of methyl groups is provided by a methyl group-donating cofactor. The natural cofactor for SET 7/9 is referred to as S-AdoMet (or AdoMet), in essence an adenosinylated methionine molecule. During the SET 7/9 HMT-catalysed reaction, the methyl group is transferred from S-AdoMet to the lysine residue of the substrate. The resulting demethylated cofactor is referred to as S-AdoHomocysteine (abbreviated as S-AdoHcy or AdoHcy). The structures of AdoMet and AdoHcy are shown in FIGS. 1a and 1b respectively.

In particular, there are several computer modelling packages commercially available which can facilitate the rational design of ligand molecules which are likely to modulate the methyltransferase activity of SET 7/9 HMT. Examples include computer programs such as GRAM, DOCK and AUTODOCK (Walters et al, 1998 Drug Discovery Today 3, 160-178; Dunbrack et al, 1997 Folding and Design 2, 2742) which can be employed, with knowledge of the ternary structure a complex comprising SET 7/9, to design potential reversible or irreversible SET 7/9 HMT inhibitors.

Alternatively a computer program may be employed to analyse the active site of SET 7/9 HMT and predict the structure of chemical moieties which will interact with the active site. An example of one such program is GRID (described by Goodford, 1985 J. Med. Chem. 28, 849-857).

In addition, the likely forces of attraction and repulsion, and the degree of any steric hindrance, between SET 7/9 HMT and a prospective ligand, can be estimated using computer programs. In general, the greater the forces of attraction (and the lower the forces of repulsion), the closer the fit and the fewer the number of steric hindrances, the tighter will be the interaction between SET 7/9 HMT and the ligand. In addition, the greater the specificity of binding of the ligand, the lower the likelihood of any adverse reactions from undesired interactions with other proteins.

Thus, in a further aspect, the invention provides a method of selecting or designing a ligand for SET 7/9, which method comprises use of at least part of the atomic co-ordinate data contained in Annex 1 (preferably a substantial part of the data i.e. the data relating to at least 50% of the atoms identified in Annex 1, more preferably most of the data i.e. the data relating to at least 75% of the atoms, and most preferably substantially all of the data i.e. the data relating to at least 95% of the atoms), or data derivable therefrom. Data derivable from the atomic co-ordinate data presented in Annex 1 include structure factor data (see Blundell et al, in “Protein Crystallography” Academic Press, New York, London and San Francisco (1976)). Where less than the complete data set is used for the modelling or designing method of the invention, it is preferred at least to include data relating to that part of the SET domain which constitutes the active site. In particular the inventors believe the following residues to be important to the catalytic activity of the SET 7/9 HMT:

Tyr 245, His 252, Hsp 256, Asn 263, Gly 264, Thr 266, Leu 267, Ser 268, Gly 292, His 293, Ala 295, Tyr 305, Lys 317, Tyr 335, and Tyr 337.

Accordingly, preferred methods of selecting or designing potential ligands will typically comprise use of atomic co-ordinate data for at least some of the atoms present in one or more (preferably most, more preferably all) of the residues identified immediately above.

Typically the method of this aspect of the invention comprises the step of modelling all or part of the structure of SET 7/9 HMT using a computer and identifying a potential ligand by designing or selecting a molecule based on its likely ability to interact with the modeled structure.

A potential ligand may be a new chemical entity, although this has the disadvantage that a method of synthesising the entity must be devised if the compound is to be tested in vitro. More preferably therefore, the potential ligand is a compound which is already available. Commercially available libraries of compound structures, such as the Cambridge Structural Database, allow for computer-based high throughput screening of compounds in order to identify and select potential ligands.

Potential ligands which have been designed or selected on the basis of computer modelling or otherwise may then be synthesised or, more preferably, obtained from commercial sources for in vitro testing. The potential ligand may be tested, for example, for any enhancing or inhibitory effect on the methyltransferase activity of SET 7/9 HMT. An assay of HMT activity is described herein and may readily be modified to investigate the effect of the ligand e.g. by contacting a preparation comprising SET 7/9 HMT with a suitable lysine-containing peptide or protein substrate and a suitable cofactor in the presence or absence of the potential ligand.

An assay of methyltransferase activity has the advantage of indicating not only whether a ligand binds to SET 7/9 HMT but also whether such binding has a modulating effect on the catalytic activity of the enzyme. However, other in vitro screening or analytical methods might usefully be employed as an alternative or as an adjunct to enzyme activity assays.

For example, the potential ligand could be labelled, conveniently with a fluorophore. Many suitable fluorophores are commercially available and include inter alia, fluorescein and rhodamine. Binding of the labelled potential ligand to SET 7/9 (alone or in complex with a substrate and/or cofactor) could then be readily detected and assayed (e.g. in a fluorescence polarization assay—see, for instance, Sokham et al, 1999 Anal. Biochem. 275, 156-161).

If desired, any complex resulting from interaction of the potential ligand with SET 7/9 HMT can be analysed to obtain detailed structural information about the binding of the (potential) ligand to SET 7/9. This allows for the structure of the selected or designed ligand to be altered in a rational way in order to optimise affinity and/or specificity of binding of the ligand to SET 7/9. In particular, if desired, a complex comprising SET 7/9 HMT and the ligand may be crystallised and subject to X-ray crystallography in order to obtain data to “fine tune” the interaction between the ligand and SET 7/9 HMT. The interaction may be optimised, for example, by adding or removing groups from the ligand, substituting groups or otherwise altering the overall shape of the ligand.

It will be appreciated that the process of computer modelling and in vitro testing and/or analysis could be performed in an iterative manner, if desired.

In another aspect the invention provides a computer readable medium comprising either (a) at least part of the atomic co-ordinate data contained within Annex 1, or (b) structure factor data derivable from at least part of the atomic co-ordinate data contained within Annex 1.

The invention also provides a computer system for the purpose of modelling structures and/or performing rational drug design of a potential ligand for the SET 7/9 HMT (alone or in complex with a substrate and/or cofactor), the system comprising either (a) at least part of the atomic co-ordinate data contained within Annex 1, or (b) structure factor data derivable from at least part of the atomic co-ordinate data contained within Annex 1.

Preferably the computer readable medium and/or computer system comprises positional data relating to, or derivable from, at least 50%, more preferably at least 75%, and most preferably at least 95% of the atoms detailed in Annex 1.

In yet another aspect the invention provides a method of obtaining a crystal comprising at least a catalytically active portion of a SET 7/9 domain, the method comprising the steps of:
(i) forming a complex comprising at least a catalytically active portion of a SET 7/9 domain with a lysine-containing substrate peptide or protein, and/or with a cofactor; and
(ii) forming a crystal comprising the complex.

More especially, the inventors have surprisingly found that optimal results are obtained if the lysine residue of the substrate which interacts with the active site of the SET 7/9 domain in the complex is methylated (especially, mono-methylated), and such represents a preferred feature of the invention. In addition, it is also preferred that the cofactor, if present in the complex, is in unmethylated form (e.g. AdoHcy).

The inventors have found that, in preferred embodiments, a crystal may be formed from a complex of a methylated lysine-containing substrate peptide or protein, a SET 7/9 domain, and an unmethylated cofactor (such as AdoHcy), which crystal diffracts X-rays so as to allow determination of the atomic co-ordinates of the SET 7/9 domain to a resolution of 1.7 Å or better.

In yet another aspect the invention provides a method of obtaining a crystal comprising at least a catalytically active portion of a SET 7/9 domain, the method comprising the steps of:
(i) forming a complex comprising at least a catalytically active portion of a SET 7/9 domain and a ligand therefor (the ligand being an inhibitor of SET 7/9 HMT activity); and
(ii) forming a crystal comprising the complex.

In the crystal-forming method aspects of the invention, the complex will typically comprise at least residues 108-366 of the SET domain, which portion is catalytically active.

In a further aspect, the invention provides a ligand for a SET 7/9 domain which modulates the methyltransferase activity thereof, said ligand having been selected or designed by analysis or modelling using at least part of the atomic co-ordinate data contained within Annex 1 or structure factor data obtainable therefrom. More specifically the ligand is typically selected or designed by analysis or modelling using data from at least 50%, more preferably at least 75% and most preferably at least 95% of the atoms detailed in Annex 1.

The ligand may, for example, be one which interacts with one or more of the residues of the SET 7/9 domain which the inventors believe to be important for the HMTase activity of the SET 7/9 domain, namely:

Tyr 245, His 252, Hsp 256, Asn 263, Gly 264, Thr 266, Leu 267, Ser 268, Gly 292, His 293, Ala 295, Tyr 305, Lys 317, Tyr 335, and Tyr 337.

Alternatively, or additionally the ligand may interact with one or more of the SET 7/9 residues identified in Table 1 above as interacting with the substrate. Preferred ligands will comprise or consist of a hydrophobic moiety which can occupy the highly hydrophobic lysine access channel, identified by the inventors in the SET 7/9 domain. Some examples of suitable ligands are given in Example 3.

Once ligands have been identified with acceptable binding specificity and affinity, they can be investigated for use as potential drugs as modulators of SET HMT activity (particularly as inhibitors). Thus, in a further aspect the invention provides a pharmaceutical composition comprising a modulator of SET HMT activity in admixture with a physiologically acceptable diluent, excipient or carrier, the modulator being a compound in accordance with general formula I or II (defined in Example 3) and/or being a modulator selected or designed by analysis or modelling using at least part of the atomic co-ordinate data contained within Annex 1 or structure factor data obtainable therefrom.

The invention also provides for use of a ligand molecule e.g. in accordance with general formula I or II, and/or selected or designed by the method of the invention as a modulator of SET HMT activity, (particularly as an inhibitor thereof). The invention additionally provides for use of a modulator of SET HMT activity to prepare a pharmaceutical composition for modulating the methylation of histones.

In addition, the data obtained by the inventors has enabled identification of key amino acid residues in the SET HMT protein which are essential to the histone methyltransferase activity of the enzyme or its specificity. Alteration of one or more of these key residues may affect the HMT properties in a useful way.

Those skilled in the art are readily able to make point mutations in proteins, provided the relevant nucleic acid sequencing encoding the protein to be mutated is available. Thus, for example, site directed mutagenesis and/or PCR could be used to introduce one or more mutations into the SET HMT protein at desired locations. Particular residues which are susceptible to mutation so as to alter HMTase activity include residues 245, 305 and 335 of the SET domain. The invention thus affords a method providing SET HMTases with altered enzyme activity, and altered SET HMTases obtained by the method.

The invention will now be further described by way of illustrative example and with reference to the accompanying drawings in which:

FIGS. 1A and 1B illustrate the structure of the SET 7/9 cofactor in its methylated (S-AdoMet) and demethylated (S-AdoHcy) states respectively;

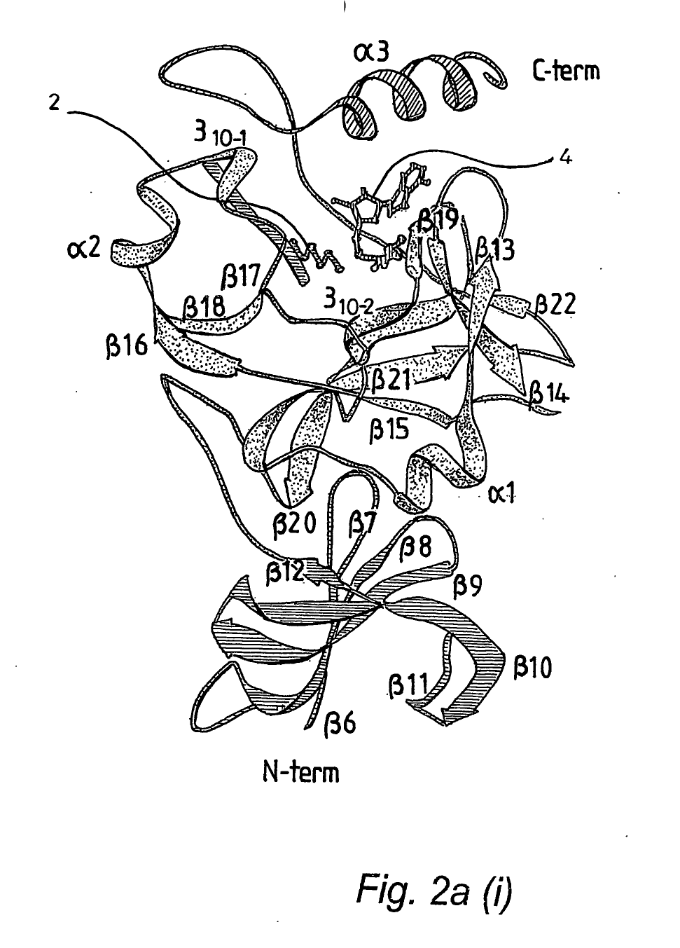

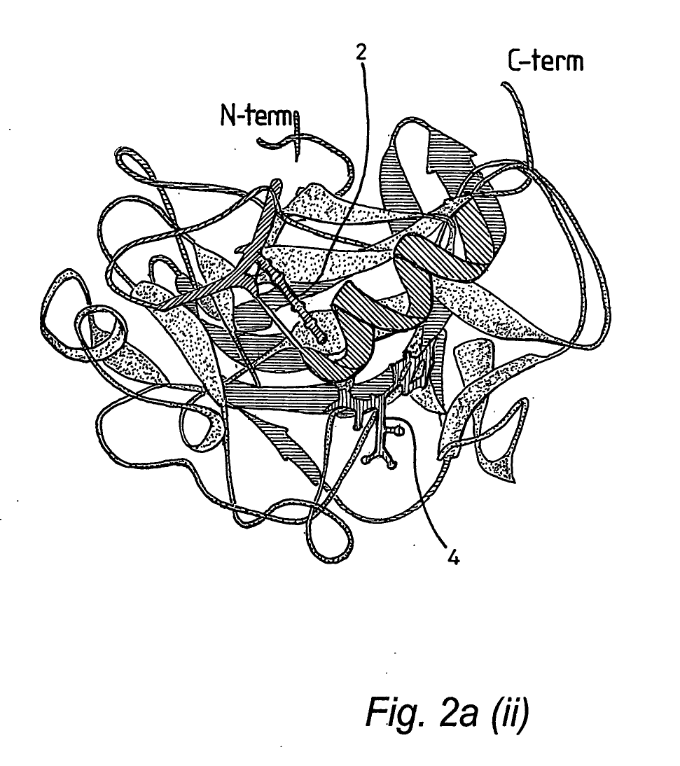

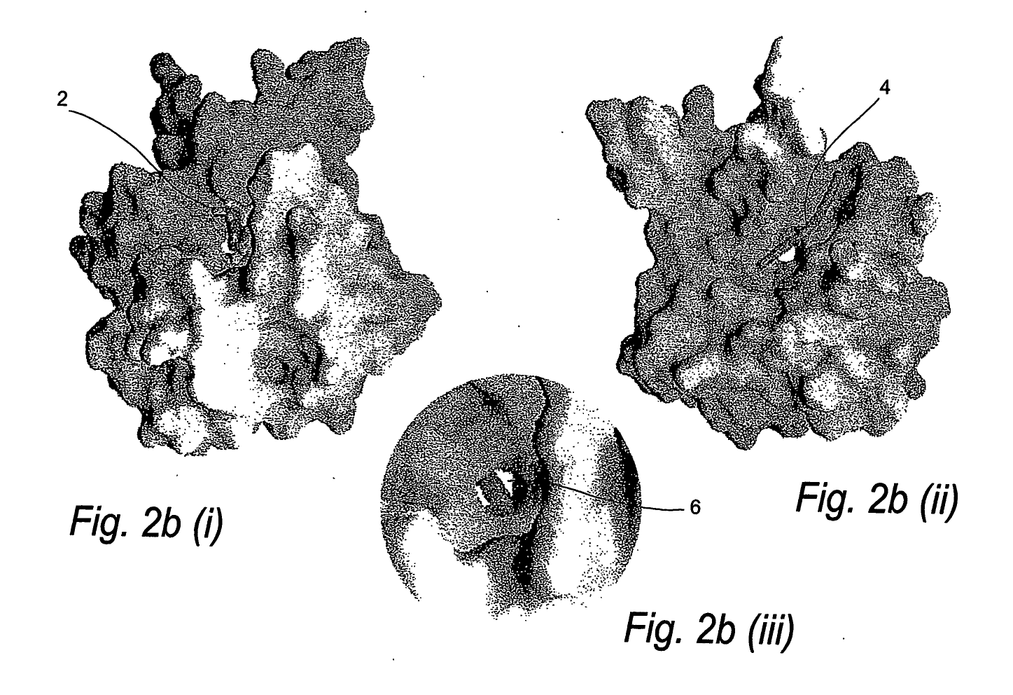

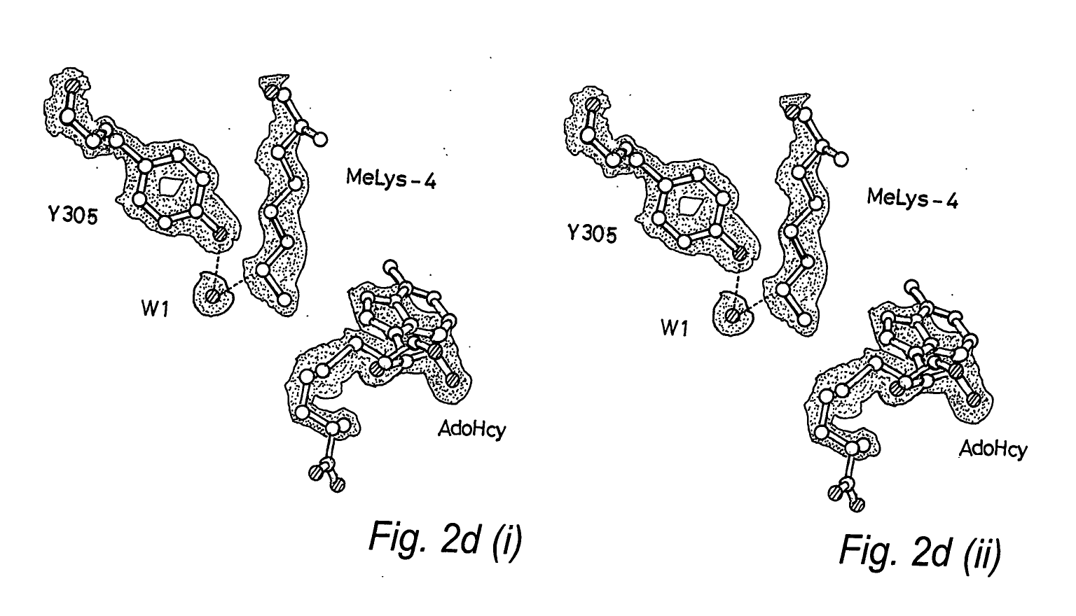

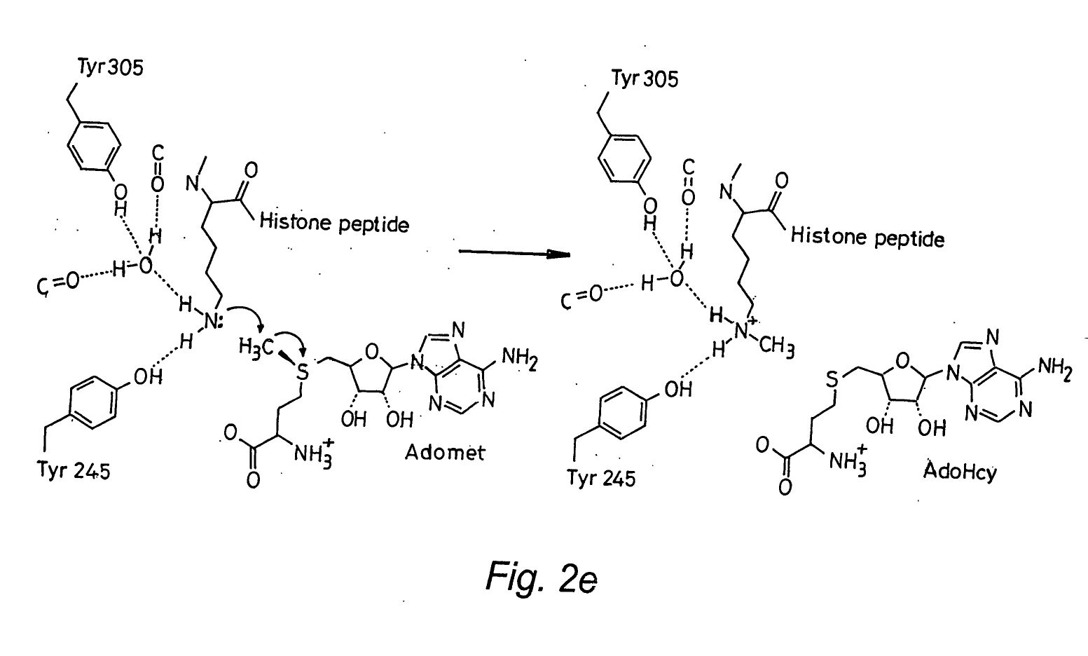

FIG. 2a(i) and 2a(ii) are two orthogonal views of the ternary structure of the SET 7/9 domain in complex with AdoHcy and a substrate peptide;

FIG. 2b(i) and 2b(ii) are two views of the SET domain using surface representation (views (i) and (ii) are related by a two-fold rotation about a vertical axis);

FIG. 2c(i)-(iv) are stereographic representations of selected active site residues of the SET 7/9 domain;

FIG. 2d(i) and (ii) are two representations of the electron density (2fo-2fc) over a small part of the active site of the SET 7/9 domain;

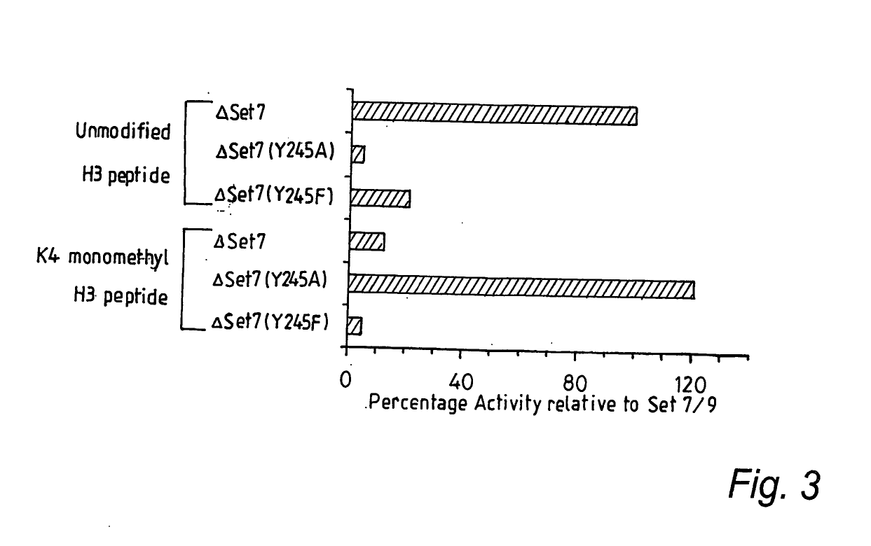

FIG. 3 is a bar chart showing the results of HMT assays of SET 7/9 and two point mutants thereof, for unmethylated or monomethylated lysine-containing peptide substrates;

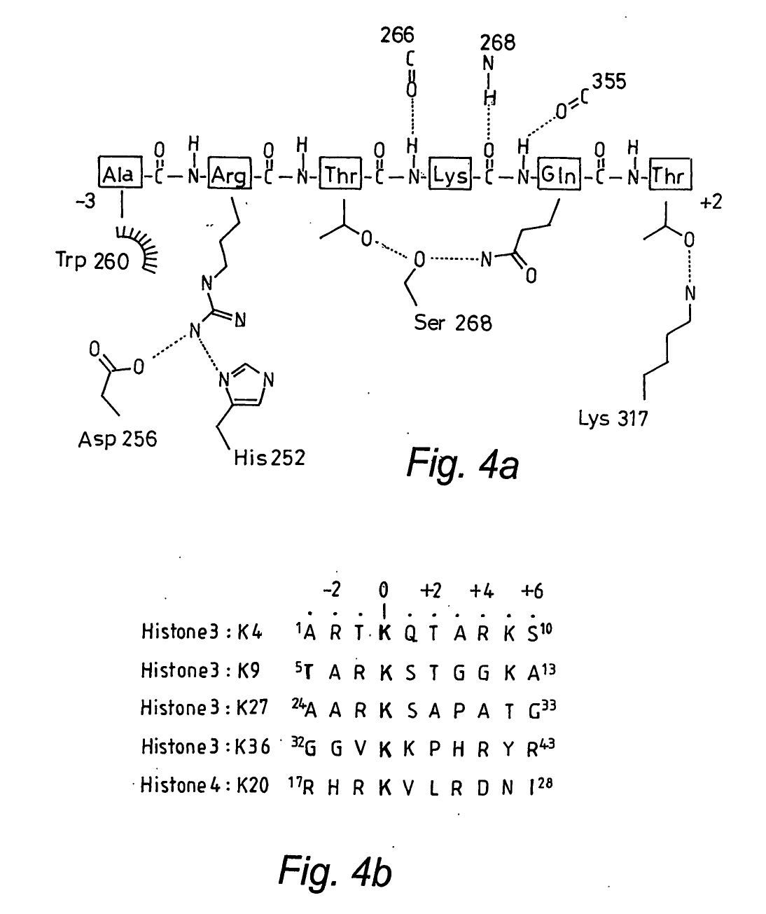

FIG. 4a is a schematic representation of various interactions made by the histone substrate peptide in complex with SET 7/9;

FIG. 4b shows the sequence of various portions of histone proteins which are known to be subject to methylation, aligned according to the position of the target lysine residues. The numbers at the top refer to the relative position of the residues with respect to the target lysine (K) residue. The numbers at the sides are the absolute positions of the residues within the various histone proteins;

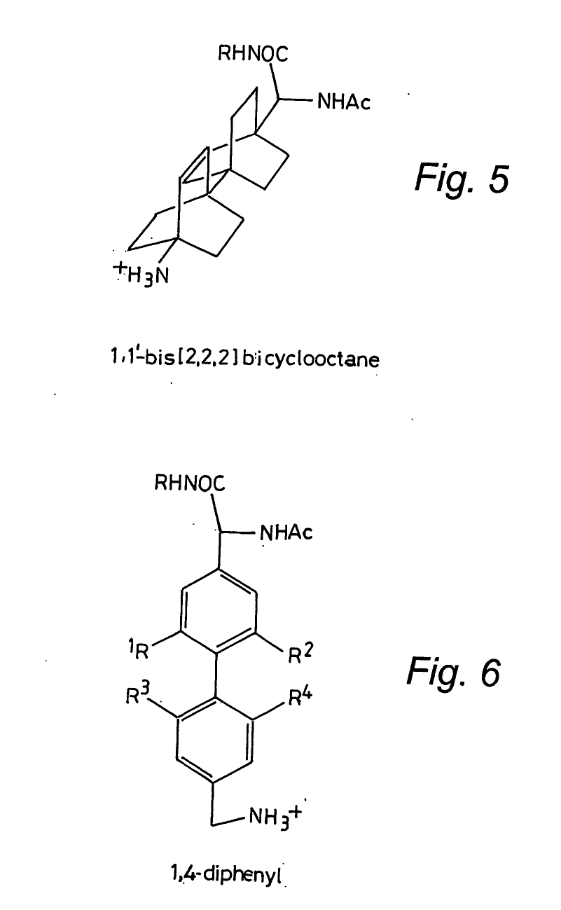

FIGS. 5 and 6 show examples of the structure of potential ligands for SET 7/9 proposed by the inventors; and

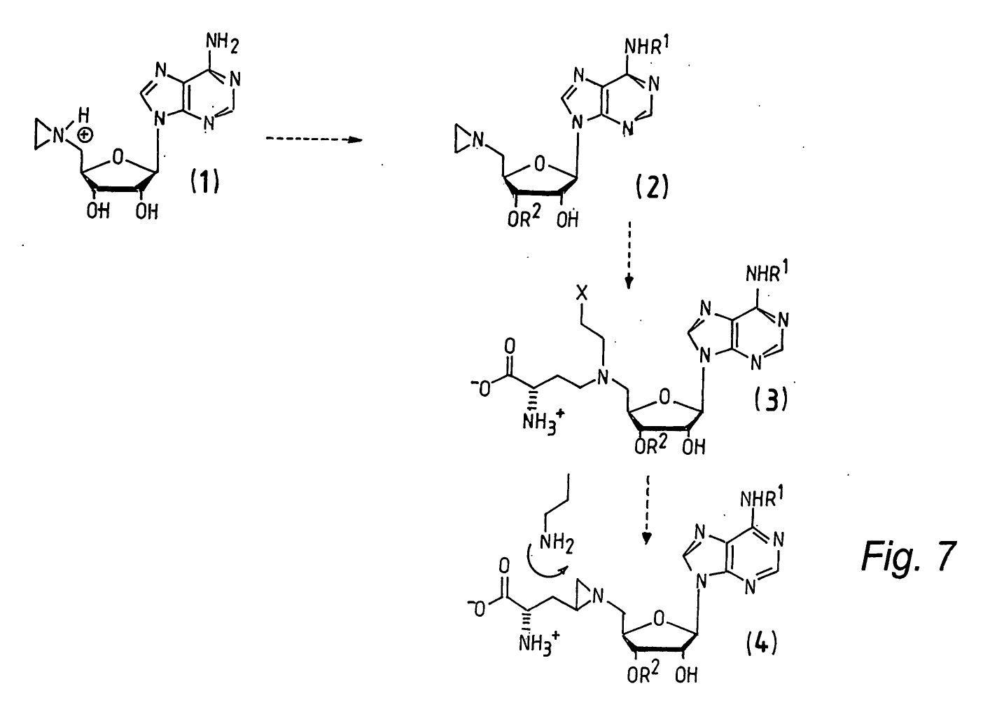

FIG. 7 is a schematic representation of a proposed synthesis scheme for preparing a further ligand of SET 7/9.

### EXAMPLES

### Example 1.1

Protein Constructs

A truncated form of the SET 7/9 HMT protein lacking the first 51 residues of the N terminal portion, referred to as ΔSET7/9 (residues 52-366), was expressed as a GST-fusion in pGEX 6P1 in E. coli BL21. The GST was removed by overnight treatment with PreScission™ Protease (Amersham) prior to gel filtration. Preparation of ΔSETT7/9 in D₂O for nmr studies resulted in a series of N-terminal degradation products which were still catalytically active. Subsequently a further truncated form of the protein, ΔΔSET7/9 (residues 108-366), was prepared as above and found to be stable for growth in D₂O and was consequently used for further nmr and crystallography experiments. Site-directed alanine mutations were introduced using the Stratagene Quikchange Mutagenesis kit, mutations were confirmed by DNA sequencing and electrospray mass spectrometry. Synthetic peptides were prepared using conventional in vitro techniques. AdoHcy was obtained from Fluka, Switzerland.

### Example 1.2

HMTase Activity Measurements

The methyltransferase activity of SET7/9 and the various mutant constructs described in the text were determined in a reaction volume of 20 μl containing 3 μM AdoMet supplemented with [methyl-³H] AdoMet (4 μCi) (Amersham Biosciences, UK) and 750 μM purified methylase in reaction buffer (50 mM Tris pH 8.5, 100 mm NaCl, 1 mM EDTA, 1 mM DTT) with 50 μM histone peptide (see below). Following incubation at 37° C. for 60 min the reaction was vacuum blotted onto membrane (Hybond-C, Amersham Biosciences, UK) washed and activity measured by scintillation counting.

### Example 1.3

Analytical Analysis of Histone Methylation

The histone methyltransferase assay for analytical purposes was carried out under slightly different conditions than those described in Example 1.2. For analytical purposes the reaction was performed at 37° C. in 50 mM Tris-HCl, pH 8.0, 100 mM NaCl, 1 mM DTT, with 300 μM AdoMet (Fluka, Switzerland), 100 μM H3 20mer peptide (ARTKQTARKSTGGKAPRKQY), and with 1.5 μM enzyme. At desired time intervals an aliquot of the reaction was removed and quenched in 8 M urea and acidified with glacial acetic acid. The reaction products were separated by reverse phase HPLC (Jasco (UK) Ltd) on a Zorbax 300SB-C18 column (Rockland Technologies, Inc. USA) using a gradient from 0 to 40% acetonitrile in the presence of 0.05% trifluoroacetic acid at 55° C. Fractions from the peptide peak were analysed using a Reflex III MALDI time-of-flight mass spectrometer (Bruker Daltonik, GmbH, Germany) to obtain positive ion mass spectra.

### Example 1.4

NMR

NMR spectra were recorded at 25° C. on a Varian Inova spectrometer operating at ¹H frequencies of 600 MHz and 800 MHz. Protein samples, 0.5 nM, were prepared in 50 mM Tris-HCl, 0.2 mM TCEP (Trialkylphosphine Tris (2-carboxyethyl)phosphine—a reducing agent), 10% D₂O, pH 6.5.

### Example 1.5

Crystallography

Protein stock solution was prepared at 100 mg/ml in 50 mM Tris, pH 7.0, 100 mM NaCl, and then incubated with a two-fold molar excess of mono-methylated Lys-410-mer peptide (ARTKQTARKS) and AdoHcy. Crystals were grown by vapour diffusion at room temperature as hanging drops. Drops were prepared by mixing equal volumes of protein complex with reservoir solution containing 0.1M Tris, pH 7.8 and 22% PEG3350.

Crystals were first transferred into mother liquor augmented with an additional 5% PEG 400, prior to plunging into liquid nitrogen. Data were collected from flash cooled crystals at 100K on an Raxis-II detector mounted on a Rigaku RU200 generator. Diffraction data were integrated and scaled using DENZO and SCALEPACK (Otwinoski & Minor in “Data Collection and Processing” (eds. Sawyer, Isaacs & Bailey) p 556-562 (SERC Daresbury Laboratory, Warrington 1993)). The crystals belong to the spacegroup P2₁, with a=52.7 Å, b=75.4 Å, c=69.1 Å and β=94.2°. The upper resolution of data collection was limited only by the minimum crystal-detector distance, for all data 20-1.75 Å the merging R factor is 0.052 with 90% completeness (0.20 and 52% for the bin, 1.8-1.75 Å); overall the data is 1-fold redundant. The structure was solved by molecular replacement using our previous model (1H3I.brk) with AMORE. Subsequent refinement was done using REFMAC5 (1994, Collaborative Computational Project 4, Acta Crystallogr. D50 p 760-763) and manual model building in 0 (Jones et al, 1991 Acta Crystallogr. A47 p 110-119). The final model comprises one AdoHcy molecule and residues 117-366 of the protein for both complexes in the asymmetric unit, for the A molecule residues 1-6 of the peptide are ordered while all 10 residues are ordered in the B molecule because of the contacts with another molecule in the lattice. At the current stage of refinement, using all data from 20-1.75 Å, there are 480 water molecules and R_{work}=0.21, R_{free}=0.24 and rms bonds=0.008.

### Example 2

### Example 2.1

Preliminary NMR Studies

Preliminary studies were performed using a complex comprising an unlabelled SET 7/9 domain (residues 52-366 of SET 7/9HMT) and a 20 amino acid residue peptide corresponding to the N terminal of histone H3, specifically labelled with ¹³C and ¹⁵N terminal of histone H3, specifically labelled with ¹³C and ¹⁵N in the lysine residue at position 4. Slightly sub-stoichiometric amounts (0.8 equivalents) of labelled peptide were used. Spectra were recorded at 25° C. using a Varian Inova spectrometer operating at ¹H frequency of 600 MHz. Studies were performed with or without overnight incubation with 5.0 equivalents of AdoMet cofactor, so as to allow comparison of methylated product and unmethylated substrate complexes.

The spectrum of the methylated product complex (data omitted for brevity) exhibited marked differences from that of the unmodified peptide, most notably a reversal of the relative populations of the Lysine-4 side-chain conformers. The relative intensities of the new peaks appearing on methylation (at 2.87, 51.4 and 2.63, 42.0 ppm) indicated that the methylated peptide is more stably bound than the unmethylated substrate and the predominant species (>90%) appeared to have an environment dissimilar to that of the free peptide. A small proportion of the H3 peptide remained unmodified.

### Example 2.2

Structural Determinations

Since the preliminary NMR studies suggested that a histone peptide containing monomethylated lysine-4 was better ordered in complex with SET 7/9 than unmodified peptide, the inventors used the products of the normal HMT reaction for crystallisation experiments (methylated lysine peptide and AdoHcy). In particular the inventors used a catalytically active construct that contains the complete C-terminal segment of the SET 7/9 domain. Well-ordered crystals of the ternary complex of SET 7/9 were obtained (using the methodology described in Example 1 and by Wilson et al, 2002 Cell 111, 105-115) that diffracted to at least 1.7 Å spacing and the structure was readily solved by molecular replacement. The C-terminal segment, the AdoHcy cofactor and most of the substrate peptide are well defined in the resulting electron density maps, as are all the important residues around the active site. The overall structure of the ternary complex of SET 7/9 is shown in two orthogonal views in FIG. 2a (i) and (ii).

FIG. 2a shows two orthogonal views [(i) and (ii)] of the SET 7/9 ternary complex using ribbon representation. The N- and C-terminals of the SET 7/9 domain are labelled “N-term” and “C-term” respectively. The side chain of the methylated lysine residue at position 4 in the substrate peptide is labelled with reference numeral 2. The demethylated AdoHcy cofactor is labelled with reference numeral 4. Elements of the secondary structure of the SET 7/9 domain are labelled (β16 etc). FIG. 2b shows two views [(I) and (ii)] of the SET 7/9 domain using surface representation, and the circular inset panel (iii) shows a magnified view of the lysine access channel (see description below) accommodating the methylated lysine side chain, as seen from the AdoHcy binding site.

FIG. 2b(i) shows the AdoHcy cofactor (2), and FIG. 2b(ii) shows the substrate peptide (4). FIG. 2b(iii) shows the lysine access channel, labelled with reference numeral 6, accommodating the lysine side chain.

The most stirring feature of the catalytic structure is that the AdoHcy and the peptide substrate are located on opposite sides of the SET domain and that there is a narrow channel (the “lysine access channel”) passing through the enzyme that connects the peptide and cofactor binding surfaces. The target lysine residue of the substrate (Lys-4) is inserted into this channel so that its amine can access the methyl donor (AdoMet). The packing of the C-terminal segment against the SET domain is required to form the lysine access channel explaining why this feature has not been observed in previous HMTase structures which did not include the C terminal segment of the SET domain (Wilson et al, 2002 Cell 111, 105-115).

The C-terminal segment (residues 345-366) is organised into two structural features; residues 337-349, belonging mainly to the SET domain, form an approximate β-hairpin structure that protrudes at a right angle to the surface of the enzyme. This is followed by three residues that accommodate a sharp bend in the polypeptide chain before the final stretch of the protein which adopts an α-helical conformation (FIG. 2a). Tyr-335 and Tyr-337, located just before the C-segment, are both important for the formation of the lysine access channel. The arrangements of the P-hairpin is such that it stabilises the conformation of these two tyrosine residues whilst also contributing to one of the sides of the groove into which the peptide binds. The second side of the peptide-binding groove is made up by residues 255-268 (including β-17). The α-helix at the end of the C-terminal segment packs against P-19, particularly Phe-299 (located just beyond the conserved NHS signature motif (Rea et al, 2000 Nature 406, 593-599), and makes hydrophobic packing interactions with the AdoHcy cofactor through Trp-352.

The mode of cofactor binding in the present structure is such that the methyl group to be transferred from the AdoMet to the amine is pointing into the lysine binding channel (see FIG. 2b). At the peptide binding site, the channel surface is largely made up by the side chains of Leu-267 and tyrosine residues -305-335 and -337 (FIGS. 2c and 2d). The alkyl component of the lysine side-chain therefore inhabits a hydrophobic environment.

The other end of the channel, where it opens onto the cofactor binding surface, is dominated by four phenoxyl hydorixdes (Tyr-245, and Tyr-305, -335, -337) and five main-chain carbonyl groups, all approximately oriented towards the lysine amine group. There are also several well defined water molecules in the vicinity of the active site, although only one is close to the lysine amine.

FIG. 2C, panels (i)-(iv), are stereographic representations of selected active site residues. The upper panels (i), (ii), show the AdoHcy and residues forming the channel occupied by the alkyl portion of the lysine side-chain. The lower panels (iii), (iv) show the arrangement of five main chain carbonyl groups around the amine group of the lysine side chain.

The arrangement is such that, along most of its length, the lysine access channel is highly hydrophobic, and thus insertion of the alkyl portion of the lysine side-chain into the channel is, energetically, very favourable. In contrast, at the far (co-factor) end, the channel is, relatively speaking, moderately hydrophilic due to the presence of the 5 carbonyl groups and this allows for accommodation of the polar amino group at the end of the lysine side-chain.

The structure, with its catalytic and binding surfaces, explains at last the role of a number of invariant residues, whose mutation to alanine has been shown to essentially abolish HMTase activity without significantly affecting the affinity for substrate or cofactor binding. The aromatic ring of Tyr-335 forms a significant part of the binding cavity for the alkyl portion of the Lys-4 residue while its hydroxyloxygen makes a single hydrogen bond to the main-chain carbonyl of residue 295. The importance of the correct positioning of this tyrosine residue is underscored by the observation that His-297 (of the NHS signature) acts to stabilise its conformation by acting as a hydrogen bond acceptor to its main-chain amide (FIG. 2c).

The electron density for the methyl-Lys-4 is very well defined and clearly shows the location of the single methyl group, FIG. 2d(i) and (ii). Examination of the active site also reveals that the lysine amine group donates hydrogen bonds to both the invariant Tyr-245 and to a tightly bound water molecule (W1). The lysine appears to be acting as a hydrogen bond donor in both cases because of the nature of the other hydrogen bonds made by Tyr-335 and W1 (FIG. 2c). The orientation of the lysine amine group is such that the amine-methyl bond is aligned towards the sulfur atom of the AdoHcy. Moreover, it is directed at sulfur along the tetrahedral vector corresponding to the (S) location of the methyl group in AdoMet (Hoffmann, 1986 Biochemistry 25, 4444-49).

FIG. 2e shows a schematic representation of the proposed reaction scheme. The structure clearly shows that the lysine has been stripped of all solvent molecules except for the one water used as a hydrogen bond acceptor to orient the amino group. This desolvation will lower the pKa of the lysine amino group and also enhance its nucleophilicity. At the same time, the local orientation of the dipoles of four main-chain carbonyl groups towards the nitrogen will stabilise the developing positive charge on that atom as the methylation reaction proceeds. There is no proximate proton acceptor to provide general base catalysis for the nucleophilic nitrogen. Mechanistically, it thus seems likely that the lysine sidechain enters the active site with difficulty in its protonated form, the passage of this cation through the channel being facilitated by the faces of the flanking tyrosines. In the active site, the desolvated lysine is deprotonated, possibly to one of the flanking tyrosine oxygens. Thereafter the methylation reaction proceeds without general base catalysis, facilitated simply by orientation of orbitals, by desolvation and by stabilisation of charge reorganisation.

The present inventors have further analysed the methylation reaction. Reaction mixtures were set up with unmodified peptide and AdoMet and conditions found where the reaction was driven essentially to completion. HPLC separation of the reaction products (results omitted for brevity) gave a peak which eluted at a different position to the unmodified peptide and MALDI analysis of this material revealed only mono-methylated peptide. These results strongly support the conclusion that SET 7/9 catalyses the addition of a single methyl group to its target lysine. The reason for SET 7/9 acting as a mono-methyltransferase is readily apparent from the crystal structure now presented. The peptide used for crystallization was synthesised using mono-methylated lysine at position 4. In the crystal structure there is very well defined electron density for this methyl group in just one position. The arrangement of the two hydrogen bond acceptor groups (for the lysine amine) provides an immediate explanation for the lack of rotation about the CE-NZ bond; Tyr-245 and W1 not only make favourable interactions that stabilise the observed rotamer, but they also preclude, on steric grounds, a methyl group in any other position. Thus the structure shows that the arrangement of protein side-chains and, indirectly, water molecules at the active site of SET 7/9 is such that it can only catalyse the addition of a single methyl group to the lysine amine.

In the light of the present findings, comparison of the sequence of other SET proteins (Lachner & Jenuwein 2002, Curr. Opinion in Cell Biol. 14, 286-298), in the vicinity of the active site, suggests why many of these enzymes catalyse di- or tri-methylation of their target lysines. Only Tyr-245 and Tyr-335 are invariant across the SET family, many other residues are highly variable. So, for example, Tyr-305 is substituted for a valine residue in SUV39H1 (a tri-methylase) and a proline in G9a (a di-methylase). Both of these substitutions would seem likely to produce a cavity at the active site that could accommodate a methyl substituent on the lysine amine.

It has previously been reported that the Y245>A mutation in SET 7/9 leads to a dramatic reduction in HMTase activity (Wilson et al, 2002 cited above). The inventors have now surprisingly found that although this mutant has greatly reduced activity with respect to an unmodified lysine substrate, it has substantial activity if assayed with mono-methylated Lys-4 substrate (see FIG. 3). All of these results taken together, suggest that it will be possible both to predict from the primary structure whether a particular SET protein has mono-, di- or tri-methylase activity and to engineer altered functionality into these enzymes.

FIG. 3 shows the results of a Histone methyltransferase assay of SET 7/9, and SET 7/9 with either of the point mutations Y245→F, using histone H3 peptide substrate either unmodified or with monomethylation of the lysine residue at position 4.

The histone peptide binds in a largely extended conformation into a shallow groove (as shown in FIG. 2b). The binding is mediated by a network of hydrogen and salt bonds involving both the main-chain and side-chains of the peptide (FIG. 4a). The target Lys-4 residue is located approximately at the centre of the defined peptide and the interactions of non-conserved SET 7/9 residues with the peptide seems to account for the enzyme's specificity. Arg(−2)_{PEP} (substrate residues are numbered relative to the target lysine) makes a salt bridge with Asp-256 and a hydrogen bond with His-252. Thr(−1)_{PEP} and Gln(+1)_{PEP} both form hydrogen bonds with Ser-268, and Thr(+2) hydrogen bonds to the side-chain of Lys-317. Arg(−2)_{PEP} therefore seems to play a particularly important role in mediating substrate specificity. Moreover, inspection of the various histone target sequences (FIG. 4b) shows that only Lys-4 of histone H3 contains a basic residue in this (−2) position. The others all have a basic residue either at the (−1) or )+1) position. Both activity measurements and peptide affinity measurements, using a peptide in which Arg(−2)_{PEP} is mutated to an alanine (data not shown), support the notion that this arginine residue contributes significantly to peptide binding to SET 7/9. The structure of the ternary complex of SET 7/9 disclosed herein will provide a good model for assessing the likely substrate specificity of other SET proteins from their primary sequence.

### Example 3

Rational Design of Ligands for SET 7/9

The determination of the structure of the SET 7/9 domain (in complex with its substrate) as disclosed herein has allowed the present inventors to identify a number of compounds which may act as ligands for the SET 7/9 domain and thus modulate the methyltransferase activity of HMTs containing the SET 7/9 domain.

In particular the inventors have identified (i) potential ligands which may bind to the SET domain ether in isolation or when the protein is in complex with a histone substrate; (ii) a second group of potential ligands which will bind to the SET domain only when the protein is in isolation. Potential ligands may be envisaged which will bind to the SET domain exclusively by covalent binding, or exclusively by non-covalent binding, or by a mixture of both covalent and non-covalent interactions.

In general, potential ligands may have some elements of structure which are analogous to the AdoMet cofactor. However, detailed knowledge of the SET structure has enabled the inventors to propose specific modifications which should improve binding affinity and/or specificity.

In general it will be desirable for the potential ligand to have a stabilised 5′ thioadenosine moiety. This can be achieved, for example, by replacing the S atom of AdoMet with N.

Such a molecule, AzaAdoMet, has been synthesized previously (1999 J. Org. Chem. 64, 7467). In addition, or as an alternative it may be advantageous to derivatise atoms which (in AdoMet) do not strongly interact with the SET protein. Examples include the 3′-OH of the ribosyl moiety and the —NH₂ group at position 4 of the adenyl moiety. Substitutions at one or more of these positions are likely to have beneficial effects on pharmacokinetics in general and on metabolic stability and/or bioavailability in particular. Examples of potential ligands may conform to the general formula I, 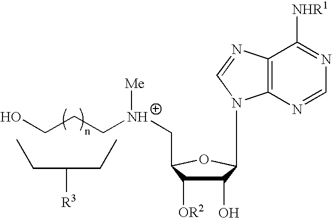
wherein R¹, R¹ and R³ are, independently: H, alkyl, haloakyl, aralkyl, acyl, cycloalkyl, alkenyl, or oxa-alkyl and wherein n=0, 1 or 2.

Preferred ligands however will comprise a hydrophobic moiety (e.g. a substituted or unsubstituted alkyl or alkenyl group) which will occupy the lysine access channel normally occupied by the side chain of the lysine residue of the substrate. Occupancy of the lysine access channel by a hydrophobic moiety of a ligand would allow for multiple strong hydrophobic interactions, providing a large binding energy for formation of the ligand/protein complex, which should be reflected in a high binding affinity. The lysine access channel is about 8 Å in diameter and, in principle, any hydrophobic moiety of suitable size might be useful for inclusion in a ligand to make use of this unusual feature of the SET 7/9 domain. Ligands of this type may, for example, conform to the general formula II, 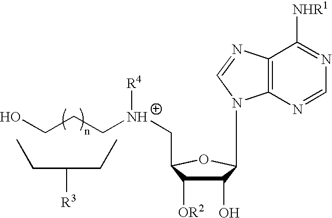
wherein R¹-R³ and are as in general formula I, and wherein R⁴ may be selected from any of the following: a linear, cyclic or branched alkyl group comprising 2 or more carbon atoms (preferably 2-6 carbon atoms, more preferably 2-5 carbon atoms); an alkenyl group having one or more double bonds; the alkyl or alkenyl group optionally being substituted at one or more positions (e.g. hydroxylated, halogenated, or aminated) and optionally comprising one or more oxa- aza- or thia-replacements of CH₂ groups.

In particular, potential ligands which might occupy the lysine access channel, and therefore bind to SET-domain containing proteins with high affinity might include 1,1′-bis [2,2,2]-bicyclooctane or 1,4-diphenyl moieties. The structures of proposed sample ligands is shown in FIGS. 5 and 6. For the diphenyl-based ligand (FIG. 6) R¹-R⁴ must be small in size, so that the diphenyl moiety can still be inserted into the lysine access channel. R¹, R¹, R³ and R⁴ will typically therefore each comprise a single atom or a small group. For example R₁-R₄ may be, independently, any one of H, F, Cl, Br, OH, methyl, Omethyl or ethyl.

A further family of potential ligands relate to an 5′-aziridinyl adenosine alkylating agent that becomes activated in the bound state as a result of protonation. The parent aziridine nucleoside has been exemplified for underivatised adenosines by E. Weinhold et al (Angew. Chem. Int. Ed., 1998, 37, 2888). An outline synthetic scheme is shown in FIG. 7. Starting from the compound (1) described in the prior art, desirable modifications (R¹, R²) could be introduced to generate species (2). Next, addition of a modified or alternated “homocysteine” group would require the previous introduction of an aziridine generating group on the 5′-nitrogen (as illustrated in species (3), where X is P, Cl, Br or Omes for example). Lastly, incorporation of the aziridine in the “homocysteine” moiety would restore the concept of drug activation by N-protonation following binding the SET-domain containing HMT. In all cases, one would expect that such a ligand would alkylate the terminal amino group of the lysine side chain of the substrate histone, “locking” it into the active site and causing essentially irreversible inhibition of the HMT.

Ligands which fill the hydrophobic lysine access channel of the SET 7/9 domain may well exhibit binding which is highly specific for histone methyltransferases and exhibiting very little, if any, binding affinity for other methyltransferases which do not possess a similar substrate access channel. Consequently, drugs based on such ligands should be similarly specific.

An alternative (and less preferred) approach would be to try to block or fill the lysine access channel from the “cofactor side” of the SET 7/9 protein, by marketing use of protein/protein interactions (e.g. by using a peptide mimetic approach), based on knowledge of the flanking amino acid residues surrounding the opening of the channel. Such peptides are readily synthesized in vitro in large quantities. Conceivably it might be possible to combine the two approaches by using a peptide mimetic with a hydrophobic moiety to be inserted into the lysine access channel—although this would need to allow for the relatively polar nature of the channel at the co-factor end.

Annex 1

| | |
|---|---|
| HEADER | TRANSFERASE 18-DEC-02 1O9S |
| TITLE | CRYSTAL STRUCTURE OF A TERNARY COMPLEX OF THE HUMAN HISTONE |
| TITLE | 2 METHYLTRANSFERASE SET7/9 |
| COMPND | MOL_ID; 1; |
| COMPND | 2 MOLECULE: HISTONE-LYSINE N-METHYLTRANSFERASE, H3 LYSINE-4 |
| COMPND | 3 SPECIFIC; |
| COMPND | 4 SYNONYM: HISTONE H3-K4 METHYLTRANSFERASE, H3-K4-HMTASE; |
| COMPND | 5 CHAIN: A; |
| COMPND | 6 FRAGMENT: N-DOMAIN, SET-DOMAIN, RESIDUES 108-366; |
| COMPND | 7 EC: 2.1.1.43; |
| COMPND | 8 ENGINEERED: YES; |
| COMPND | 9 MOL_ID: 2; |
| COMPND | 10 MOLECULE: GENE FRAGMENT FOR HISTONE H3; |
| COMPND | 11 CHAIN: K; |
| COMPND | 12 FRAGMENT: RESIDUES 2-11; |
| COMPND | 13 ENGINEERED: YES |
| SOURCE | MOL_ID: 1; |
| SOURCE | 2 ORGANISM_SCIENTIFIC: HOMO SAPIENS; |
| SOURCE | 3 ORGANISM_COMMON: HUMAN; |
| SOURCE | 4 EXPRESSION_SYSTEM: ESCHERICHIA COLI; |
| SOURCE | 5 MOL_ID: 2; |
| SOURCE | 6 SYNTHETIC: YES; |
| SOURCE | 7 ORGANISM_SCIENTIFIC: HOMO SAPIENS; |
| SOURCE | 8 ORGANISM_COMMON: HUMAN |
| KEYWDS | METHYLATION, HISTONE H3, METHYLTRANSFERASE, TRANSFERASE |
| EXPDTA | X-RAY DIFFRACTION |
| AUTHOR | B. XIAO, C. JING, J. R. WILSON, P. A. WALKER, N. VASISHT, G. KELLY, |
| AUTHOR | 2 S. HOWELL, I. A. TAYLOR, G. M. BLACKBURN, S. J. GAMBLIN |
| REVDAT | 1 19-DEC-02 1O9S 0 |
| JRNL | AUTH B. XIAO, C. JING, J. R. WILSON, P. A. WALKER, N. VASISHT, |
| JRNL | AUTH 2 G. KELLY, S. HOWELL, I. A. TAYLOR, G. M. BLACKBURN, |
| JRNL | AUTH 3 S. J. GAMBLIN |
| JRNL | TITL STRUCTURE AND CATALYTIC MECHANISM OF THE HUMAN |
| JRNL | TITL 2 HISTONE METHYLTRANSFERASE SET7/9 |
| JRNL | REF TO BE PUBLISHED |
| JRNL | REFN |
| REMARK | 2 RESOLUTION. 1.75 ANGSTROMS. |
| REMARK | E 501 SAH S-Adenosyl-homocyteine |
| CRYST1 | 52.690 75.438 69.099 90.00 94.16 90.00 P 1 21 1 4 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.018979 | 0.000000 | 0.001380 | 0.00000 |
| SCALE2 | 0.000000 | 0.013256 | 0.000000 | 0.00000 |
| SCALE3 | 0.000000 | 0.000000 | 0.014510 | 0.00000 |

| | No. | Type | Residue | | | X | Y | Z | Occ. | B | |
|---|---|---|---|---|---|---|---|---|---|---|---|
| ATOM | 1 | N | GLY | A | 117 | 31.852 | −2.694 | 2.451 | 1.00 | 13.90 | N |
| ATOM | 3 | CA | GLY | A | 117 | 30.788 | −1.711 | 2.826 | 1.00 | 13.55 | C |
| ATOM | 6 | C | GLY | A | 117 | 30.367 | −1.598 | 4.276 | 1.00 | 13.14 | C |
| ATOM | 7 | O | GLY | A | 117 | 29.481 | −0.788 | 4.552 | 1.00 | 14.03 | O |
| ATOM | 10 | N | VAL | A | 118 | 30.820 | −2.492 | 5.146 | 1.00 | 11.64 | N |
| ATOM | 12 | CA | VAL | A | 118 | 30.552 | −2.429 | 6.568 | 1.00 | 10.23 | C |
| ATOM | 14 | CB | VAL | A | 118 | 31.408 | −3.477 | 7.277 | 1.00 | 10.37 | C |
| ATOM | 16 | CG1 | VAL | A | 118 | 30.860 | −3.776 | 8.647 | 1.00 | 10.40 | C |
| ATOM | 20 | CG2 | VAL | A | 118 | 31.444 | −4.758 | 6.440 | 1.00 | 10.29 | C |
| ATOM | 24 | C | VAL | A | 118 | 30.691 | −1.045 | 7.215 | 1.00 | 9.12 | C |
| ATOM | 25 | O | VAL | A | 118 | 31.738 | −0.387 | 7.133 | 1.00 | 8.38 | [symbol] |
| ATOM | 26 | N | CYS | A | 119 | 29.609 | −0.620 | 7.860 | 1.00 | 7.88 | [symbol] |
| ATOM | 28 | CA | CYS | A | 119 | 29.560 | 0.642 | 8.582 | 1.00 | 7.63 | [symbol] |
| ATOM | 30 | CB | CYS | A | 119 | 28.553 | 1.595 | 7.946 | 1.00 | 7.90 | [symbol] |
| ATOM | 33 | SG | CYS | A | 119 | 28.136 | 3.044 | 8.935 | 1.00 | 8.87 | [symbol] |
| ATOM | 34 | C | CYS | A | 119 | 29.191 | 0.387 | 10.047 | 1.00 | 7.19 | [symbol] |
| ATOM | 35 | O | CYS | A | 119 | 28.248 | −0.351 | 10.346 | 1.00 | 6.63 | [symbol] |
| ATOM | 36 | N | TRP | A | 120 | 29.963 | 0.979 | 10.949 | 1.00 | 6.31 | [symbol] |
| ATOM | 38 | CA | TRP | A | 120 | 29.692 | 0.897 | 12.381 | 1.00 | 6.30 | [symbol] |
| ATOM | 40 | CB | TRP | A | 120 | 30.960 | 0.559 | 13.183 | 1.00 | 6.44 | [symbol] |
| ATOM | 43 | CG | TRP | A | 120 | 31.500 | −0.810 | 12.987 | 1.00 | 6.89 | [symbol] |
| ATOM | 44 | CD1 | TRP | A | 120 | 32.219 | −1.265 | 11.920 | 1.00 | 7.26 | [symbol] |
| ATOM | 46 | NE1 | TRP | A | 120 | 32.574 | −2.580 | 12.117 | 1.00 | 8.17 | [symbol] |
| ATOM | 48 | CE2 | TRP | A | 120 | 32.088 | −2.997 | 13.327 | 1.00 | 8.70 | [symbol] |
| ATOM | 49 | CD2 | TRP | A | 120 | 31.416 | −1.902 | 13.908 | 1.00 | 7.94 | [symbol] |
| ATOM | 50 | CE3 | TRP | A | 120 | 30.827 | −2.073 | 15.164 | 1.00 | 8.69 | [symbol] |
| ATOM | 52 | CZ3 | TRP | A | 120 | 30.926 | −3.312 | 15.792 | 1.00 | 9.60 | [symbol] |
| ATOM | 54 | CH2 | TRP | A | 120 | 31.604 | −4.378 | 15.188 | 1.00 | 9.83 | [symbol] |
| ATOM | 56 | CZ2 | TRP | A | 120 | 32.196 | −4.240 | 13.963 | 1.00 | 9.55 | [symbol] |
| ATOM | 58 | C | TRP | A | 120 | 29.190 | 2.262 | 12.847 | 1.00 | 6.01 | [symbol] |
| ATOM | 59 | O | TRP | A | 120 | 29.838 | 3.279 | 12.602 | 1.00 | 5.78 | [symbol] |
| ATOM | 60 | N | ILE | A | 121 | 28.033 | 2.284 | 13.495 | 1.00 | 5.92 | [symbol] |
| ATOM | 62 | CA | ILE | A | 121 | 27.493 | 3.517 | 14.047 | 1.00 | 6.04 | [symbol] |
| ATOM | 64 | CB | ILE | A | 121 | 26.105 | 3.817 | 13.469 | 1.00 | 6.35 | [symbol] |
| ATOM | 66 | CG1 | ILE | A | 121 | 26.154 | 3.835 | 11.936 | 1.00 | 6.79 | [symbol] |
| ATOM | 69 | CD1 | ILE | A | 121 | 24.786 | 4.013 | 11.282 | 1.00 | 7.65 | [symbol] |
| ATOM | 73 | CG2 | ILE | A | 121 | 25.600 | 5.165 | 13.962 | 1.00 | 6.50 | [symbol] |
| ATOM | 77 | C | ILE | A | 121 | 27.420 | 3.373 | 15.574 | 1.00 | 5.80 | [symbol] |
| ATOM | 78 | O | ILE | A | 121 | 26.590 | 2.634 | 16.101 | 1.00 | 5.54 | [symbol] |
| ATOM | 79 | N | TYR | A | 122 | 28.304 | 4.064 | 16.278 | 1.00 | 5.71 | [symbol] |
| ATOM | 81 | CA | TYR | A | 122 | 28.300 | 4.007 | 17.733 | 1.00 | 5.65 | [symbol] |
| ATOM | 83 | CB | TYR | A | 122 | 29.719 | 4.092 | 18.288 | 1.00 | 5.69 | [symbol] |
| ATOM | 86 | CG | TYR | A | 122 | 30.466 | 2.790 | 18.211 | 1.00 | 5.87 | [symbol] |
| ATOM | 87 | CD1 | TYR | A | 122 | 31.219 | 2.470 | 17.089 | 1.00 | 6.06 | [symbol] |
| ATOM | 89 | CE1 | TYR | A | 122 | 31.900 | 1.285 | 17.007 | 1.00 | 6.49 | [symbol] |
| ATOM | 91 | CZ | TYR | A | 122 | 31.842 | 0.396 | 18.055 | 1.00 | 6.75 | [symbol] |
| ATOM | 92 | OH | TYR | A | 122 | 32.531 | −0.783 | 17.973 | 1.00 | 9.10 | [symbol] |
| ATOM | 94 | CE2 | TYR | A | 122 | 31.106 | 0.683 | 19.184 | 1.00 | 6.65 | [symbol] |
| ATOM | 96 | CD2 | TYR | A | 122 | 30.420 | 1.877 | 19.257 | 1.00 | 5.78 | [symbol] |
| ATOM | 98 | C | TYR | A | 122 | 27.479 | 5.132 | 18.319 | 1.00 | 5.62 | [symbol] |
| ATOM | 99 | O | TYR | A | 122 | 27.680 | 6.304 | 17.993 | 1.00 | 6.01 | [symbol] |
| ATOM | 100 | N | TYR | A | 123 | 26.551 | 4.773 | 19.190 | 1.00 | 5.73 | [symbol] |
| ATOM | 102 | CA | TYR | A | 123 | 25.770 | 5.769 | 19.901 | 1.00 | 6.09 | [symbol] |
| ATOM | 104 | CB | TYR | A | 123 | 24.457 | 5.180 | 20.405 | 1.00 | 6.43 | [symbol] |
| ATOM | 107 | CG | TYR | A | 123 | 23.532 | 4.727 | 19.308 | 1.00 | 7.37 | [symbol] |
| ATOM | 108 | CD1 | TYR | A | 123 | 23.343 | 3.381 | 19.055 | 1.00 | 8.49 | [symbol] |
| ATOM | 110 | CE1 | TYR | A | 123 | 22.488 | 2.952 | 18.050 | 1.00 | 8.58 | [symbol] |
| ATOM | 112 | CZ | TYR | A | 123 | 21.815 | 3.883 | 17.284 | 1.00 | 9.47 | [symbol] |
| ATOM | 113 | OH | TYR | A | 123 | 20.969 | 3.448 | 16.296 | 1.00 | 11.11 | [symbol] |
| ATOM | 115 | CE2 | TYR | A | 123 | 21.984 | 5.234 | 17.515 | 1.00 | 8.85 | [symbol] |
| ATOM | 117 | CD2 | TYR | A | 123 | 22.840 | 5.648 | 18.528 | 1.00 | 8.33 | [symbol] |
| ATOM | 119 | C | TYR | A | 123 | 26.603 | 6.258 | 21.080 | 1.00 | 5.50 | [symbol] |
| ATOM | 120 | O | TYR | A | 123 | 27.512 | 5.561 | 21.556 | 1.00 | 5.44 | [symbol] |
| ATOM | 121 | N | PRO | A | 124 | 26.303 | 7.453 | 21.564 | 1.00 | 5.04 | [symbol] |
| ATOM | 122 | CA | PRO | A | 124 | 27.026 | 8.012 | 22.709 | 1.00 | 4.56 | [symbol] |
| ATOM | 124 | CB | PRO | A | 124 | 26.311 | 9.349 | 22.970 | 1.00 | 4.62 | [symbol] |
| ATOM | 127 | CG | PRO | A | 124 | 25.365 | 9.565 | 21.870 | 1.00 | 5.23 | [symbol] |
| ATOM | 130 | CD | PRO | A | 124 | 25.265 | 8.357 | 21.059 | 1.00 | 5.10 | [symbol] |
| ATOM | 133 | C | PRO | A | 124 | 26.979 | 7.113 | 23.955 | 1.00 | 3.98 | [symbol] |
| ATOM | 134 | O | PRO | A | 124 | 27.880 | 7.201 | 24.781 | 1.00 | 3.93 | [symbol] |
| ATOM | 135 | N | ASP | A | 125 | 25.966 | 6.257 | 24.066 | 1.00 | 3.55 | [symbol] |
| ATOM | 137 | CA | ASP | A | 125 | 25.821 | 5.359 | 25.207 | 1.00 | 3.08 | [symbol] |
| ATOM | 139 | CB | ASP | A | 125 | 24.365 | 4.953 | 25.422 | 1.00 | 2.63 | [symbol] |
| ATOM | 142 | CG | ASP | A | 125 | 23.731 | 4.358 | 24.181 | 1.00 | 2.85 | [symbol] |
| ATOM | 143 | OD1 | ASP | A | 125 | 24.275 | 3.374 | 23.635 | 1.00 | 2.64 | [symbol] |
| ATOM | 144 | OD2 | ASP | A | 125 | 22.675 | 4.814 | 23.706 | 1.00 | 3.02 | [symbol] |
| ATOM | 145 | C | ASP | A | 125 | 26.691 | 4.110 | 25.104 | 1.00 | 3.21 | [symbol] |
| ATOM | 146 | O | ASP | A | 125 | 26.774 | 3.342 | 26.056 | 1.00 | 2.98 | [symbol] |
| ATOM | 147 | N | GLY | A | 126 | 27.320 | 3.888 | 23.955 | 1.00 | 3.70 | [symbol] |
| ATOM | 149 | CA | GLY | A | 126 | 28.228 | 2.756 | 23.814 | 1.00 | 3.59 | [symbol] |
| ATOM | 152 | C | GLY | A | 126 | 27.684 | 1.636 | 22.955 | 1.00 | 3.82 | [symbol] |
| ATOM | 153 | O | GLY | A | 126 | 28.433 | 0.769 | 22.515 | 1.00 | 3.55 | [symbol] |
| ATOM | 154 | N | GLY | A | 127 | 26.371 | 1.642 | 22.718 | 1.00 | 3.88 | N |
| ATOM | 156 | CA | GLY | A | 127 | 25.761 | 0.659 | 21.840 | 1.00 | 4.21 | [symbol] |
| ATOM | 159 | C | GLY | A | 127 | 26.096 | 1.018 | 20.395 | 1.00 | 4.61 | [symbol] |
| ATOM | 160 | O | GLY | A | 127 | 26.593 | 2.109 | 20.109 | 1.00 | 4.48 | [symbol] |
| ATOM | 161 | N | SER | A | 128 | 25.825 | 0.110 | 19.470 | 1.00 | 5.37 | [symbol] |
| ATOM | 163 | CA | SER | A | 128 | 26.140 | 0.372 | 18.070 | 1.00 | 5.88 | [symbol] |
| ATOM | 165 | CB | SER | A | 128 | 27.619 | 0.028 | 17.808 | 1.00 | 6.36 | [symbol] |
| ATOM | 168 | OG | SER | A | 128 | 27.904 | −1.320 | 18.139 | 1.00 | 6.52 | [symbol] |
| ATOM | 170 | C | SER | A | 128 | 25.253 | −0.376 | 17.094 | 1.00 | 6.17 | [symbol] |
| ATOM | 171 | O | SER | A | 128 | 24.612 | −1.363 | 17.446 | 1.00 | 5.68 | [symbol] |
| ATOM | 172 | N | LEU | A | 129 | 25.203 | 0.150 | 15.870 | 1.00 | 6.68 | [symbol] |
| ATOM | 174 | CA | LEU | A | 129 | 24.546 | −0.488 | 14.742 | 1.00 | 7.41 | [symbol] |
| ATOM | 176 | CB | LEU | A | 129 | 23.546 | 0.469 | 14.084 | 1.00 | 7.78 | [symbol] |
| ATOM | 179 | CG | LEU | A | 129 | 22.059 | 0.098 | 14.150 | 1.00 | 10.11 | [symbol] |
| ATOM | 181 | CD1 | LEU | A | 129 | 21.609 | −0.271 | 15.535 | 1.00 | 11.82 | [symbol] |
| ATOM | 185 | CD2 | LEU | A | 129 | 21.209 | 1.256 | 13.588 | 1.00 | 10.96 | [symbol] |
| ATOM | 189 | C | LEU | A | 129 | 25.678 | −0.852 | 13.776 | 1.00 | 7.31 | [symbol] |
| ATOM | 190 | O | LEU | A | 129 | 26.581 | −0.041 | 13.520 | 1.00 | 7.01 | [symbol] |
| ATOM | 191 | N | VAL | A | 130 | 25.652 | −2.075 | 13.256 | 1.00 | 7.45 | [symbol] |
| ATOM | 193 | CA | VAL | A | 130 | 26.715 | −2.540 | 12.373 | 1.00 | 7.62 | [symbol] |
| ATOM | 195 | CB | VAL | A | 130 | 27.722 | −3.377 | 13.136 | 1.00 | 7.81 | [symbol] |
| ATOM | 197 | CG1 | VAL | A | 130 | 28.953 | −3.618 | 12.265 | 1.00 | 7.47 | [symbol] |
| ATOM | 201 | CG2 | VAL | A | 130 | 28.079 | −2.689 | 14.417 | 1.00 | 8.87 | [symbol] |
| ATOM | 205 | C | VAL | A | 130 | 26.200 | −3.410 | 11.245 | 1.00 | 7.84 | [symbol] |
| ATOM | 206 | O | VAL | A | 130 | 25.346 | −4.265 | 11.448 | 1.00 | 8.27 | [symbol] |
| ATOM | 207 | N | GLY | A | 131 | 26.740 | −3.196 | 10.055 | 1.00 | 7.83 | [symbol] |
| ATOM | 209 | CA | GLY | A | 131 | 26.349 | −3.988 | 8.903 | 1.00 | 7.72 | [symbol] |
| ATOM | 212 | C | GLY | A | 131 | 26.744 | −3.336 | 7.597 | 1.00 | 8.01 | [symbol] |
| ATOM | 213 | O | GLY | A | 131 | 27.115 | −2.155 | 7.536 | 1.00 | 6.88 | [symbol] |
| ATOM | 214 | N | GLU | A | 132 | 26.693 | −4.139 | 6.545 | 1.00 | 8.56 | [symbol] |
| ATOM | 216 | CA | GLU | A | 132 | 26.931 | −3.650 | 5.207 | 1.00 | 9.76 | [symbol] |
| ATOM | 218 | CB | GLU | A | 132 | 26.773 | −4.795 | 4.209 | 1.00 | 10.61 | [symbol] |
| ATOM | 221 | CG | GLU | A | 132 | 28.057 | −5.375 | 3.662 | 1.00 | 12.77 | [symbol] |
| ATOM | 224 | CD | GLU | A | 132 | 27.834 | −6.071 | 2.335 | 1.00 | 14.90 | [symbol] |
| ATOM | 225 | OE1 | GLU | A | 132 | 26.692 | −6.069 | 1.849 | 1.00 | 17.36 | [symbol] |
| ATOM | 226 | OE2 | GLU | A | 132 | 28.804 | −6.614 | 1.768 | 1.00 | 16.80 | [symbol] |
| ATOM | 227 | C | GLU | A | 132 | 25.855 | −2.630 | 4.884 | 1.00 | 9.88 | [symbol] |
| ATOM | 228 | O | GLU | A | 132 | 24.685 | −2.877 | 5.150 | 1.00 | 10.33 | [symbol] |
| ATOM | 229 | N | VAL | A | 133 | 26.231 | −1.495 | 4.310 | 1.00 | 10.07 | [symbol] |
| ATOM | 231 | CA | VAL | A | 133 | 25.234 | −0.516 | 3.848 | 1.00 | 10.25 | [symbol] |
| ATOM | 233 | CB | VAL | A | 133 | 25.797 | 0.910 | 3.870 | 1.00 | 10.52 | [symbol] |
| ATOM | 235 | CG1 | VAL | A | 133 | 26.278 | 1.237 | 5.252 | 1.00 | 10.72 | [symbol] |
| ATOM | 239 | CG2 | VAL | A | 133 | 26.920 | 1.082 | 2.851 | 1.00 | 10.68 | [symbol] |
| ATOM | 243 | C | VAL | A | 133 | 24.729 | −0.853 | 2.433 | 1.00 | 10.42 | [symbol] |
| ATOM | 244 | O | VAL | A | 133 | 25.398 | −1.575 | 1.695 | 1.00 | 9.26 | [symbol] |
| ATOM | 245 | N | ASN | A | 134 | 23.541 | −0.339 | 2.077 | 1.00 | 10.94 | [symbol] |
| ATOM | 247 | CA | ASN | A | 134 | 22.936 | −0.558 | 0.756 | 1.00 | 11.92 | [symbol] |
| ATOM | 249 | CB | ASN | A | 134 | 21.446 | −0.102 | 0.768 | 1.00 | 11.89 | [symbol] |
| ATOM | 252 | CG | AASN | A | 134 | 20.496 | −1.066 | 0.269 | 0.50 | 13.75 | [symbol] |
| ATOM | 253 | CG | BASN | A | 134 | 21.305 | 1.409 | 0.958 | 0.50 | 12.62 | [symbol] |
| ATOM | 254 | OD1 | AASN | A | 134 | 20.857 | −2.146 | −0.162 | 0.50 | 15.50 | [symbol] |
| ATOM | 255 | OD1 | BASN | A | 134 | 22.289 | 2.136 | 1.015 | 0.50 | 13.62 | [symbol] |
| ATOM | 256 | ND2 | AASN | A | 134 | 19.235 | −0.632 | 0.183 | 0.50 | 14.57 | [symbol] |
| ATOM | 257 | ND2 | BASN | A | 134 | 20.070 | 1.880 | 1.015 | 0.50 | 13.29 | [symbol] |
| ATOM | 262 | C | ASN | A | 134 | 23.704 | 0.253 | −0.293 | 1.00 | 12.39 | [symbol] |
| ATOM | 263 | O | ASN | A | 134 | 24.599 | 1.030 | 0.041 | 1.00 | 11.91 | [symbol] |
| ATOM | 264 | N | GLU | A | 135 | 23.340 | 0.089 | −1.563 | 1.00 | 13.28 | [symbol] |
| ATOM | 266 | CA | GLU | A | 135 | 23.973 | 0.862 | −2.630 | 1.00 | 14.12 | [symbol] |
| ATOM | 268 | CB | GLU | A | 135 | 23.433 | 0.475 | −4.026 | 1.00 | 14.45 | [symbol] |
| ATOM | 271 | CG | GLU | A | 135 | 21.955 | 0.726 | −4.294 | 1.00 | 15.44 | [symbol] |
| ATOM | 274 | CD | GLU | A | 135 | 21.568 | 0.407 | −5.735 | 1.00 | 16.94 | [symbol] |
| ATOM | 275 | OE1 | GLU | A | 135 | 22.468 | 0.069 | −6.538 | 1.00 | 17.74 | [symbol] |
| ATOM | 276 | OE2 | GLU | A | 135 | 20.366 | 0.501 | −6.085 | 1.00 | 17.47 | [symbol] |
| ATOM | 277 | C | GLU | A | 135 | 23.812 | 2.368 | −2.347 | 1.00 | 14.41 | [symbol] |
| ATOM | 278 | O | GLU | A | 135 | 24.527 | 3.190 | −2.918 | 1.00 | 14.88 | [symbol] |
| ATOM | 279 | N | ASP | A | 136 | 22.890 | 2.713 | −1.446 | 1.00 | 14.52 | [symbol] |
| ATOM | 281 | CA | ASP | A | 136 | 22.604 | 4.105 | −1.083 | 1.00 | 14.64 | [symbol] |
| ATOM | 283 | CB | ASP | A | 136 | 21.090 | 4.344 | −1.066 | 1.00 | 14.86 | [symbol] |
| ATOM | 286 | CG | ASP | A | 136 | 20.460 | 4.271 | −2.443 | 1.00 | 15.26 | [symbol] |
| ATOM | 287 | OD1 | ASP | A | 136 | 21.185 | 4.369 | −3.456 | 1.00 | 15.80 | [symbol] |
| ATOM | 288 | OD2 | ASP | A | 136 | 19.233 | 4.120 | −2.607 | 1.00 | 15.74 | [symbol] |
| ATOM | 289 | C | ASP | A | 136 | 23.153 | 4.501 | 0.291 | 1.00 | 14.53 | [symbol] |
| ATOM | 290 | O | ASP | A | 136 | 22.784 | 5.548 | 0.825 | 1.00 | 14.88 | [symbol] |
| ATOM | 291 | N | GLY | A | 137 | 24.008 | 3.666 | 0.876 | 1.00 | 14.11 | [symbol] |
| ATOM | 293 | CA | GLY | A | 137 | 24.585 | 3.960 | 2.179 | 1.00 | 13.69 | [symbol] |
| ATOM | 296 | C | GLY | A | 137 | 23.634 | 3.851 | 3.361 | 1.00 | 13.26 | [symbol] |
| ATOM | 297 | O | GLY | A | 137 | 23.916 | 4.401 | 4.427 | 1.00 | 13.56 | [symbol] |
| ATOM | 298 | N | GLU | A | 138 | 22.520 | 3.142 | 3.197 | 1.00 | 12.56 | [symbol] |
| ATOM | 300 | CA | GLU | A | 138 | 21.573 | 2.951 | 4.302 | 1.00 | 12.13 | [symbol] |
| ATOM | 302 | CB | GLU | A | 138 | 20.132 | 3.043 | 3.807 | 1.00 | 12.20 | [symbol] |
| ATOM | 305 | CG | GLU | A | 138 | 19.762 | 4.404 | 3.243 | 1.00 | 13.10 | [symbol] |
| ATOM | 308 | CD | GLU | A | 138 | 18.564 | 4.341 | 2.320 | 1.00 | 14.32 | [symbol] |
| ATOM | 309 | OE1 | GLU | A | 138 | 18.166 | 3.219 | 1.942 | 1.00 | 15.05 | [symbol] |
| ATOM | 310 | OE2 | GLU | A | 138 | 18.026 | 5.410 | 1.957 | 1.00 | 15.58 | [symbol] |
| ATOM | 311 | C | GLU | A | 138 | 21.793 | 1.602 | 4.988 | 1.00 | 11.37 | [symbol] |
| ATOM | 312 | O | GLU | A | 138 | 22.238 | 0.648 | 4.354 | 1.00 | 11.03 | [symbol] |
| ATOM | 313 | N | MET | A | 139 | 21.489 | 1.528 | 6.285 | 1.00 | 10.72 | [symbol] |
| ATOM | 315 | CA | MET | A | 139 | 21.592 | 0.284 | 7.038 | 1.00 | 10.35 | [symbol] |
| ATOM | 317 | CB | MET | A | 139 | 21.672 | 0.584 | 8.529 | 1.00 | 11.29 | [symbol] |
| ATOM | 320 | CG | MET | A | 139 | 22.928 | 1.315 | 8.887 | 1.00 | 14.10 | [symbol] |
| ATOM | 323 | SD | MET | A | 139 | 24.340 | 0.197 | 8.839 | 1.00 | 20.90 | [symbol] |
| ATOM | 324 | CE | MET | A | 139 | 24.057 | −0.735 | 10.285 | 1.00 | 20.60 | [symbol] |
| ATOM | 328 | C | MET | A | 139 | 20.399 | −0.617 | 6.787 | 1.00 | 9.24 | [symbol] |
| ATOM | 329 | O | MET | A | 139 | 19.541 | −0.779 | 7.652 | 1.00 | 8.26 | [symbol] |
| ATOM | 330 | N | THR | A | 140 | 20.360 | −1.203 | 5.592 | 1.00 | 8.20 | [symbol] |
| ATOM | 332 | CA | THR | A | 140 | 19.276 | −2.065 | 5.171 | 1.00 | 7.66 | [symbol] |
| ATOM | 334 | CB | THR | A | 140 | 18.526 | −1.378 | 4.024 | 1.00 | 7.49 | [symbol] |
| ATOM | 336 | OG1 | THR | A | 140 | 18.095 | −0.077 | 4.443 | 1.00 | 7.44 | [symbol] |
| ATOM | 338 | CG2 | THR | A | 140 | 17.242 | −2.114 | 3.657 | 1.00 | 7.50 | [symbol] |
| ATOM | 342 | C | THR | A | 140 | 19.836 | −3.369 | 4.643 | 1.00 | 7.44 | [symbol] |
| ATOM | 343 | O | THR | A | 140 | 20.634 | −3.367 | 3.717 | 1.00 | 7.48 | [symbol] |
| ATOM | 344 | N | GLY | A | 141 | 19.409 | −4.482 | 5.222 | 1.00 | 7.61 | [symbol] |
| ATOM | 346 | CA | GLY | A | 141 | 19.866 | −5.789 | 4.789 | 1.00 | 7.57 | [symbol] |
| ATOM | 349 | C | GLY | A | 141 | 19.420 | −6.844 | 5.782 | 1.00 | 7.70 | [symbol] |
| ATOM | 350 | O | GLY | A | 141 | 18.803 | −6.509 | 6.800 | 1.00 | 7.59 | [symbol] |
| ATOM | 351 | N | GLU | A | 142 | 19.767 | −8.102 | 5.513 | 1.00 | 7.72 | [symbol] |
| ATOM | 353 | CA | GLU | A | 142 | 19.361 | −9.232 | 6.356 | 1.00 | 8.07 | [symbol] |
| ATOM | 355 | CB | GLU | A | 142 | 19.056 | −10.442 | 5.475 | 1.00 | 8.60 | [symbol] |
| ATOM | 358 | CG | GLU | A | 142 | 17.878 | −10.230 | 4.541 | 1.00 | 10.48 | [symbol] |
| ATOM | 361 | CD | GLO | A | 142 | 16.545 | −10.378 | 5.242 | 1.00 | 12.69 | [symbol] |
| ATOM | 362 | OE1 | GLU | A | 142 | 16.516 | −10.311 | 6.485 | 1.00 | 14.31 | [symbol] |
| ATOM | 363 | OE2 | GLU | A | 142 | 15.527 | −10.561 | 4.545 | 1.00 | 14.94 | [symbol] |
| ATOM | 364 | C | GLU | A | 142 | 20.414 | −9.641 | 7.367 | 1.00 | 7.91 | [symbol] |
| ATOM | 365 | O | GLU | A | 142 | 20.180 | −10.523 | 8.193 | 1.00 | 7.61 | [symbol] |
| ATOM | 366 | N | LYS | A | 143 | 21.576 | −9.010 | 7.288 | 1.00 | 7.58 | [symbol] |
| ATOM | 368 | CA | LYS | A | 143 | 22.684 | −9.353 | 8.160 | 1.00 | 7.56 | [symbol] |
| ATOM | 370 | CB | LYS | A | 143 | 23.808 | −9.985 | 7.341 | 1.00 | 7.63 | [symbol] |
| ATOM | 373 | CG | LYS | A | 143 | 23.429 | −11.335 | 6.723 | 1.00 | 7.75 | [symbol] |
| ATOM | 376 | CD | LYS | A | 143 | 24.554 | −11.902 | 5.871 | 1.00 | 8.89 | [symbol] |
| ATOM | 379 | CE | LYS | A | 143 | 24.185 | −13.242 | 5.262 | 1.00 | 10.51 | [symbol] |
| ATOM | 382 | NZ | LYS | A | 143 | 25.306 | −13.829 | 4.466 | 1.00 | 11.82 | [symbol] |
| ATOM | 386 | C | LYS | A | 143 | 23.183 | −8.116 | 8.892 | 1.00 | 7.45 | [symbol] |
| ATOM | 387 | O | LYS | A | 143 | 24.385 | −7.887 | 8.988 | 1.00 | 7.79 | [symbol] |
| ATOM | 388 | N | ILE | A | 144 | 22.256 | −7.316 | 9.407 | 1.00 | 7.24 | N |
| ATOM | 390 | CA | ILE | A | 144 | 22.617 | −6.111 | 10.157 | 1.00 | 7.37 | C |
| ATOM | 392 | CB | ILE | A | 144 | 21.758 | −4.926 | 9.725 | 1.00 | 7.95 | C |
| ATOM | 394 | CG1 | ILE | A | 144 | 22.066 | −4.586 | 8.260 | 1.00 | 9.50 | C |
| ATOM | 397 | CD1 | ILE | A | 144 | 21.303 | −3.432 | 7.744 | 1.00 | 10.75 | C |
| ATOM | 401 | CG2 | ILE | A | 144 | 21.998 | −3.722 | 10.610 | 1.00 | 7.98 | C |
| ATOM | 405 | C | ILE | A | 144 | 22.461 | −6.412 | 11.643 | 1.00 | 7.00 | C |
| ATOM | 406 | O | ILE | A | 144 | 21.740 | −7.333 | 12.008 | 1.00 | 6.71 | C |
| ATOM | 407 | N | ALA | A | 145 | 23.161 | −5.675 | 12.505 | 1.00 | 6.55 | N |
| ATOM | 409 | CA | ALA | A | 145 | 23.060 | −5.947 | 13.924 | 1.00 | 6.38 | C |
| ATOM | 411 | CB | ALA | A | 145 | 24.182 | −6.892 | 14.344 | 1.00 | 6.56 | C |
| ATOM | 415 | C | ALA | A | 145 | 23.103 | −4.720 | 14.822 | 1.00 | 6.21 | C |
| ATOM | 416 | O | ALA | A | 145 | 23.744 | −3.709 | 14.515 | 1.00 | 6.27 | O |
| ATOM | 417 | N | TYR | A | 146 | 22.397 | −4.821 | 15.935 | 1.00 | 5.83 | N |
| ATOM | 419 | CA | TYR | A | 146 | 22.567 | −3.855 | 16.991 | 1.00 | 5.75 | C |
| ATOM | 421 | CB | TYR | A | 146 | 21.278 | −3.491 | 17.680 | 1.00 | 5.65 | C |
| ATOM | 424 | CG | TYR | A | 146 | 21.556 | −2.722 | 18.955 | 1.00 | 5.17 | C |
| ATOM | 425 | CD1 | TYR | A | 146 | 21.855 | −1.366 | 18.922 | 1.00 | 4.51 | C |
| ATOM | 427 | CE1 | TYR | A | 146 | 22.124 | −0.661 | 20.096 | 1.00 | 4.27 | C |
| ATOM | 429 | CZ | TYR | A | 146 | 22.107 | −1.322 | 21.306 | 1.00 | 4.15 | C |
| ATOM | 430 | OH | TYR | A | 146 | 22.373 | −0.623 | 22.469 | 1.00 | 3.48 | O |
| ATOM | 432 | CE2 | TYR | A | 146 | 21.825 | −2.671 | 21.359 | 1.00 | 4.42 | C |
| ATOM | 434 | CD2 | TYR | A | 146 | 21.566 | −3.364 | 20.193 | 1.00 | 4.81 | C |
| ATOM | 436 | C | TYR | A | 146 | 23.451 | −4.543 | 18.015 | 1.00 | 5.68 | C |
| ATOM | 437 | O | TYR | A | 146 | 23.214 | −5.695 | 18.365 | 1.00 | 5.97 | O |
| ATOM | 438 | N | VAL | A | 147 | 24.458 | −3.845 | 18.509 | 1.00 | 5.56 | N |
| ATOM | 440 | CA | VAL | A | 147 | 25.326 | −4.434 | 19.509 | 1.00 | 5.28 | C |
| ATOM | 442 | CB | VAL | A | 147 | 26.791 | −4.503 | 19.025 | 1.00 | 5.60 | C |
| ATOM | 444 | CG1 | VAL | A | 147 | 27.644 | −5.214 | 20.036 | 1.00 | 5.40 | C |
| ATOM | 448 | CG2 | VAL | A | 147 | 26.869 | −5.217 | 17.673 | 1.00 | 6.04 | C |
| ATOM | 452 | C | VAL | A | 147 | 25.213 | −3.592 | 20.781 | 1.00 | 4.80 | C |
| ATOM | 453 | O | VAL | A | 147 | 25.357 | −2.370 | 20.735 | 1.00 | 4.27 | O |
| ATOM | 454 | N | TYR | A | 148 | 24.933 | −4.249 | 21.902 | 1.00 | 4.48 | N |
| ATOM | 456 | CA | TYR | A | 148 | 24.817 | −3.575 | 23.194 | 1.00 | 4.13 | C |
| ATOM | 458 | CB | TYR | A | 148 | 24.291 | −4.555 | 24.243 | 1.00 | 3.94 | C |
| ATOM | 461 | CG | TYR | A | 148 | 22.795 | −4.859 | 24.136 | 1.00 | 3.58 | C |
| ATOM | 462 | CD1 | TYR | A | 148 | 22.327 | −5.880 | 23.325 | 1.00 | 3.66 | C |
| ATOM | 464 | CE1 | TYR | A | 148 | 20.983 | −6.167 | 23.248 | 1.00 | 4.23 | C |
| ATOM | 466 | CZ | TYR | A | 148 | 20.076 | −5.422 | 23.975 | 1.00 | 3.79 | C |
| ATOM | 467 | OH | TYR | A | 148 | 18.740 | −5.721 | 23.900 | 1.00 | 4.39 | O |
| ATOM | 469 | CE2 | TYR | A | 148 | 20.515 | −4.400 | 24.791 | 1.00 | 3.42 | C |
| ATOM | 471 | CD2 | TYR | A | 148 | 21.869 | −4.132 | 24.865 | 1.00 | 3.02 | C |
| ATOM | 473 | C | TYR | A | 148 | 26.181 | −2.994 | 23.603 | 1.00 | 3.93 | C |
| ATOM | 474 | O | TYR | A | 148 | 27.193 | −3.334 | 22.992 | 1.00 | 4.16 | O |
| ATOM | 475 | N | PRO | A | 149 | 26.216 | −2.117 | 24.614 | 1.00 | 3.52 | N |
| ATOM | 476 | CA | PRO | A | 149 | 27.463 | −1.488 | 25.059 | 1.00 | 3.71 | C |
| ATOM | 478 | CB | PRO | A | 149 | 26.999 | −0.534 | 26.166 | 1.00 | 3.63 | C |
| ATOM | 481 | CG | PRO | A | 149 | 25.556 | −0.323 | 25.932 | 1.00 | 3.10 | C |
| ATOM | 484 | CD | PRO | A | 149 | 25.068 | −1.619 | 25.385 | 1.00 | 3.36 | C |
| ATOM | 487 | C | PRO | A | 149 | 28.520 | −2.429 | 25.607 | 1.00 | 3.72 | C |
| ATOM | 488 | O | PRO | A | 149 | 29.644 | −1.979 | 25.827 | 1.00 | 3.15 | O |
| ATOM | 489 | N | ASP | A | 150 | 28.195 | −3.698 | 25.820 | 1.00 | 4.10 | N |
| ATOM | 491 | CA | ASP | A | 150 | 29.219 | −4.647 | 26.257 | 1.00 | 4.16 | C |
| ATOM | 493 | CB | ASP | A | 150 | 28.632 | −5.834 | 27.034 | 1.00 | 4.16 | C |
| ATOM | 496 | CG | ASP | A | 150 | 27.697 | −6.702 | 26.218 | 1.00 | 4.50 | C |
| ATOM | 497 | OD1 | ASP | A | 150 | 27.395 | −6.381 | 25.041 | 1.00 | 4.01 | O |
| ATOM | 498 | OD2 | ASP | A | 150 | 27.199 | −7.741 | 26.711 | 1.00 | 4.84 | O |
| ATOM | 499 | C | ASP | A | 150 | 30.088 | −5.102 | 25.073 | 1.00 | 4.57 | C |
| ATOM | 500 | O | ASP | A | 150 | 31.110 | −5.769 | 25.277 | 1.00 | 4.32 | O |
| ATOM | 501 | N | GLU | A | 151 | 29.710 | −4.680 | 23.863 | 1.00 | 4.71 | N |
| ATOM | 503 | CA | GLU | A | 151 | 30.388 | −5.043 | 22.617 | 1.00 | 5.51 | C |
| ATOM | 505 | CB | GLU | A | 151 | 31.796 | −4.464 | 22.555 | 1.00 | 6.19 | C |
| ATOM | 508 | CG | GLU | A | 151 | 31.809 | −2.948 | 22.701 | 1.00 | 7.98 | C |
| ATOM | 511 | CD | GLU | A | 151 | 33.198 | −2.358 | 22.605 | 1.00 | 9.90 | C |
| ATOM | 512 | OE1 | GLU | A | 151 | 33.909 | −2.350 | 23.625 | 1.00 | 11.04 | [symbol] |
| ATOM | 513 | OE2 | GLU | A | 151 | 33.570 | −1.893 | 21.510 | 1.00 | 12.19 | [symbol] |
| ATOM | 514 | C | GLU | A | 151 | 30.408 | −6.554 | 22.441 | 1.00 | 5.71 | [symbol] |
| ATOM | 515 | O | GLU | A | 151 | 31.357 | −7.117 | 21.909 | 1.00 | 5.94 | [symbol] |
| ATOM | 516 | N | ARG | A | 152 | 29.336 | −7.208 | 22.872 | 1.00 | 5.92 | [symbol] |
| ATOM | 518 | CA | ARG | A | 152 | 29.271 | −8.663 | 22.846 | 1.00 | 6.09 | [symbol] |
| ATOM | 520 | CB | ARG | A | 152 | 29.723 | −9.216 | 24.206 | 1.00 | 6.65 | [symbol] |
| ATOM | 523 | CG | ARG | A | 152 | 29.715 | −10.730 | 24.288 | 1.00 | 7.72 | [symbol] |
| ATOM | 526 | CD | ARG | A | 152 | 30.177 | −11.289 | 25.644 | 1.00 | 9.71 | [symbol] |
| ATOM | 529 | NE | ARG | A | 152 | 30.019 | −12.741 | 25.683 | 1.00 | 11.70 | [symbol] |
| ATOM | 531 | CZ | ARG | A | 152 | 31.007 | −13.605 | 25.870 | 1.00 | 13.50 | [symbol] |
| ATOM | 532 | NH1 | ARG | A | 152 | 32.247 | −13.183 | 26.085 | 1.00 | 14.88 | [symbol] |
| ATOM | 535 | NH2 | ARG | A | 152 | 30.753 | −14.906 | 25.867 | 1.00 | 14.21 | [symbol] |
| ATOM | 538 | C | ARG | A | 152 | 27.870 | −9.156 | 22.530 | 1.00 | 5.87 | [symbol] |
| ATOM | 539 | O | ARG | A | 152 | 27.682 | −9.962 | 21.623 | 1.00 | 6.13 | [symbol] |
| ATOM | 540 | N | THR | A | 153 | 26.890 | −8.686 | 23.291 | 1.00 | 5.66 | [symbol] |
| ATOM | 542 | CA | THR | A | 153 | 25.507 | −9.066 | 23.066 | 1.00 | 5.27 | [symbol] |
| ATOM | 544 | CB | THR | A | 153 | 24.656 | −8.749 | 24.274 | 1.00 | 5.42 | [symbol] |
| ATOM | 546 | OG1 | THR | A | 153 | 25.280 | −9.288 | 25.445 | 1.00 | 6.02 | [symbol] |
| ATOM | 548 | CG2 | THR | A | 153 | 23.311 | −9.467 | 24.197 | 1.00 | 5.91 | [symbol] |
| ATOM | 552 | C | THR | A | 153 | 24.984 | −8.289 | 21.884 | 1.00 | 5.05 | [symbol] |
| ATOM | 553 | O | THR | A | 153 | 25.111 | −7.060 | 21.842 | 1.00 | 4.35 | [symbol] |
| ATOM | 554 | N | ALA | A | 154 | 24.373 | −9.010 | 20.947 | 1.00 | 4.76 | [symbol] |
| ATOM | 556 | CA | ALA | A | 154 | 23.892 | −8.413 | 19.723 | 1.00 | 4.85 | [symbol] |
| ATOM | 558 | CB | ALA | A | 154 | 24.885 | −8.678 | 18.600 | 1.00 | 4.69 | [symbol] |
| ATOM | 562 | C | ALA | A | 154 | 22.514 | −8.935 | 19.326 | 1.00 | 4.90 | [symbol] |
| ATOM | 563 | O | ALA | A | 154 | 22.090 | −10.016 | 19.740 | 1.00 | 5.00 | [symbol] |
| ATOM | 564 | N | LEU | A | 155 | 21.838 | −8.137 | 18.508 | 1.00 | 5.04 | [symbol] |
| ATOM | 566 | CA | LEU | A | 155 | 20.543 | −8.475 | 17.934 | 1.00 | 4.96 | [symbol] |
| ATOM | 568 | CB | LEU | A | 155 | 19.488 | −7.453 | 18.363 | 1.00 | 4.60 | [symbol] |
| ATOM | 571 | CG | LEU | A | 155 | 19.311 | −7.227 | 19.871 | 1.00 | 4.30 | [symbol] |
| ATOM | 573 | CD1 | LEU | A | 155 | 18.176 | −6.271 | 20.143 | 1.00 | 4.42 | [symbol] |
| ATOM | 577 | CD2 | LEU | A | 155 | 19.082 | −8.530 | 20.607 | 1.00 | 3.93 | [symbol] |
| ATOM | 581 | C | LEU | A | 155 | 20.792 | −8.437 | 16.420 | 1.00 | 5.23 | [symbol] |
| ATOM | 582 | O | LEU | A | 155 | 20.928 | −7.370 | 15.836 | 1.00 | 5.43 | [symbol] |
| ATOM | 583 | N | TYR | A | 156 | 20.866 | −9.613 | 15.805 | 1.00 | 5.87 | [symbol] |
| ATOM | 585 | CA | TYR | A | 156 | 21.312 | −9.782 | 14.422 | 1.00 | 6.34 | [symbol] |
| ATOM | 587 | CB | TYR | A | 156 | 22.454 | −10.815 | 14.445 | 1.00 | 6.89 | [symbol] |
| ATOM | 590 | CG | TYR | A | 156 | 23.081 | −11.169 | 13.117 | 1.00 | 8.47 | [symbol] |
| ATOM | 591 | CD1 | TYR | A | 156 | 24.005 | −10.336 | 12.519 | 1.00 | 9.81 | [symbol] |
| ATOM | 593 | CE1 | TYR | A | 156 | 24.604 | −10.665 | 11.332 | 1.00 | 9.89 | [symbol] |
| ATOM | 595 | CZ | TYR | A | 156 | 24.300 | −11.848 | 10.728 | 1.00 | 10.98 | [symbol] |
| ATOM | 596 | OH | TYR | A | 156 | 24.892 | −12.163 | 9.533 | 1.00 | 11.95 | [symbol] |
| ATOM | 598 | CE2 | TYR | A | 156 | 23.380 | −12.703 | 11.297 | 1.00 | 11.43 | [symbol] |
| ATOM | 600 | CD2 | TYR | A | 156 | 22.790 | −12.368 | 12.495 | 1.00 | 10.67 | [symbol] |
| ATOM | 602 | C | TYR | A | 156 | 20.205 | −10.268 | 13.497 | 1.00 | 6.17 | [symbol] |
| ATOM | 603 | O | TYR | A | 156 | 19.532 | −11.251 | 13.798 | 1.00 | 5.91 | [symbol] |
| ATOM | 604 | N | GLY | A | 157 | 20.027 | −9.588 | 12.362 | 1.00 | 5.92 | [symbol] |
| ATOM | 606 | CA | GLY | A | 157 | 19.010 | −9.977 | 11.407 | 1.00 | 6.02 | [symbol] |
| ATOM | 609 | C | GLY | A | 157 | 18.607 | −8.857 | 10.467 | 1.00 | 5.97 | [symbol] |
| ATOM | 610 | O | GLY | A | 157 | 19.444 | −8.093 | 9.979 | 1.00 | 5.47 | [symbol] |
| ATOM | 611 | N | LYS | A | 158 | 17.304 | −8.754 | 10.251 | 1.00 | 6.58 | [symbol] |
| ATOM | 613 | CA | LYS | A | 158 | 16.730 | −7.820 | 9.289 | 1.00 | 6.79 | [symbol] |
| ATOM | 615 | CB | LYS | A | 158 | 15.383 | −8.366 | 8.800 | 1.00 | 7.28 | [symbol] |
| ATOM | 618 | CG | LYS | A | 158 | 14.762 | −7.570 | 7.673 | 1.00 | 8.22 | [symbol] |
| ATOM | 621 | CD | LYS | A | 158 | 13.496 | −8.225 | 7.123 | 1.00 | 9.35 | [symbol] |
| ATOM | 624 | CE | LYS | A | 158 | 12.982 | −7.512 | 5.882 | 1.00 | 10.69 | [symbol] |
| ATOM | 627 | NZ | LYS | A | 158 | 14.055 | −7.155 | 4.906 | 1.00 | 11.82 | [symbol] |
| ATOM | 631 | C | LYS | A | 158 | 16.539 | −6.421 | 9.862 | 1.00 | 6.67 | [symbol] |
| ATOM | 632 | O | LYS | A | 158 | 15.893 | −6.245 | 10.888 | 1.00 | 7.00 | [symbol] |
| ATOM | 633 | N | PHE | A | 159 | 17.132 | −5.435 | 9.199 | 1.00 | 6.57 | [symbol] |
| ATOM | 635 | CA | PHE | A | 159 | 16.961 | −4.033 | 9.558 | 1.00 | 6.42 | [symbol] |
| ATOM | 637 | CB | PHE | A | 159 | 18.244 | −3.424 | 10.113 | 1.00 | 6.39 | [symbol] |
| ATOM | 640 | CG | PHE | A | 159 | 18.557 | −3.810 | 11.527 | 1.00 | 6.21 | [symbol] |
| ATOM | 641 | CD1 | PHE | A | 159 | 18.938 | −5.105 | 11.834 | 1.00 | 6.41 | [symbol] |
| ATOM | 643 | CE1 | PHE | A | 159 | 19.258 | −5.462 | 13.124 | 1.00 | 7.30 | [symbol] |
| ATOM | 645 | CZ | PHE | A | 159 | 19.205 | −4.522 | 14.141 | 1.00 | 7.03 | [symbol] |
| ATOM | 647 | CE2 | PHE | A | 159 | 18.840 | −3.225 | 13.861 | 1.00 | 6.65 | [symbol] |
| ATOM | 649 | CD2 | PHE | A | 159 | 18.510 | −2.865 | 12.549 | 1.00 | 6.66 | [symbol] |
| ATOM | 651 | C | PHE | A | 159 | 16.630 | −3.272 | 8.288 | 1.00 | 6.42 | [symbol] |
| ATOM | 652 | O | PHE | A | 159 | 17.075 | −3.655 | 7.206 | 1.00 | 6.21 | [symbol] |
| ATOM | 653 | N | ILE | A | 160 | 15.872 | −2.189 | 8.423 | 1.00 | 6.70 | [symbol] |
| ATOM | 655 | CA | ILE | A | 160 | 15.584 | −1.310 | 7.295 | 1.00 | 6.91 | [symbol] |
| ATOM | 657 | CB | ILE | A | 160 | 14.128 | −1.421 | 6.841 | 1.00 | 6.96 | [symbol] |
| ATOM | 659 | CG1 | ILE | A | 160 | 13.857 | −2.802 | 6.238 | 1.00 | 6.91 | [symbol] |
| ATOM | 662 | CD1 | ILE | A | 160 | 12.379 | −3.105 | 6.032 | 1.00 | 7.38 | [symbol] |
| ATOM | 666 | CG2 | ILE | A | 160 | 13.854 | −0.364 | 5.804 | 1.00 | 6.76 | [symbol] |
| ATOM | 670 | C | ILE | A | 160 | 15.891 | 0.123 | 7.731 | 1.00 | 7.39 | [symbol] |
| ATOM | 671 | O | ILE | A | 160 | 15.271 | 0.635 | 8.663 | 1.00 | 7.44 | [symbol] |
| ATOM | 672 | N | ASP | A | 161 | 16.870 | 0.747 | 7.084 | 1.00 | 7.95 | [symbol] |
| ATOM | 674 | CA | ASP | A | 161 | 17.286 | 2.105 | 7.420 | 1.00 | 8.37 | [symbol] |
| ATOM | 676 | CB | ASP | A | 161 | 16.195 | 3.102 | 7.053 | 1.00 | 8.59 | [symbol] |
| ATOM | 679 | CG | ASP | A | 161 | 16.708 | 4.528 | 6.967 | 1.00 | 10.23 | [symbol] |
| ATOM | 680 | OD1 | ASP | A | 161 | 17.935 | 4.715 | 6.802 | 1.00 | 11.11 | [symbol] |
| ATOM | 681 | OD2 | ASP | A | 161 | 15.951 | 5.527 | 7.038 | 1.00 | 11.88 | [symbol] |
| ATOM | 682 | C | ASP | A | 161 | 17.607 | 2.256 | 8.905 | 1.00 | 8.18 | [symbol] |
| ATOM | 683 | O | ASP | A | 161 | 17.222 | 3.248 | 9.508 | 1.00 | 8.44 | [symbol] |
| ATOM | 684 | N | GLY | A | 162 | 18.292 | 1.272 | 9.486 | 1.00 | 8.28 | [symbol] |
| ATOM | 686 | CA | GLY | A | 162 | 18.703 | 1.321 | 10.888 | 1.00 | 7.95 | [symbol] |
| ATOM | 689 | C | GLY | A | 162 | 17.660 | 0.834 | 11.883 | 1.00 | 7.65 | [symbol] |
| ATOM | 690 | O | GLY | A | 162 | 17.938 | 0.718 | 13.080 | 1.00 | 7.80 | [symbol] |
| ATOM | 691 | N | GLU | A | 163 | 16.464 | 0.540 | 11.390 | 1.00 | 7.22 | [symbol] |
| ATOM | 693 | CA | GLU | A | 163 | 15.357 | 0.112 | 12.228 | 1.00 | 7.06 | [symbol] |
| ATOM | 695 | CB | GLU | A | 163 | 14.042 | 0.667 | 11.683 | 1.00 | 7.80 | [symbol] |
| ATOM | 698 | CG | GLU | A | 163 | 12.835 | 0.348 | 12.546 | 1.00 | 9.55 | [symbol] |
| ATOM | 701 | CD | GLU | A | 163 | 11.566 | 0.978 | 12.014 | 1.00 | 12.39 | C |
| ATOM | 702 | OE1 | GLU | A | 163 | 10.581 | 0.247 | 11.786 | 1.00 | 13.95 | O |
| ATOM | 703 | OE2 | GLU | A | 163 | 11.561 | 2.208 | 11.819 | 1.00 | 15.05 | O |
| ATOM | 704 | C | GLU | A | 163 | 15.312 | −1.408 | 12.280 | 1.00 | 6.25 | C |
| ATOM | 705 | O | GLU | A | 163 | 15.342 | −2.067 | 11.245 | 1.00 | 6.04 | O |
| ATOM | 706 | N | MET | A | 164 | 15.232 | −1.956 | 13.488 | 1.00 | 4.98 | N |
| ATOM | 708 | CA | MET | A | 164 | 15.210 | −3.396 | 13.688 | 1.00 | 4.77 | C |
| ATOM | 710 | CB | MET | A | 164 | 15.531 | −3.723 | 15.143 | 1.00 | 4.72 | C |
| ATOM | 713 | CG | MET | A | 164 | 15.774 | −5.202 | 15.382 | 1.00 | 4.94 | C |
| ATOM | 716 | SD | MET | A | 164 | 16.451 | −5.533 | 17.027 | 1.00 | 5.80 | S |
| ATOM | 717 | CE | MET | A | 164 | 15.147 | −4.981 | 17.997 | 1.00 | 5.96 | C |
| ATOM | 721 | C | MET | A | 164 | 13.870 | −4.029 | 13.329 | 1.00 | 4.84 | C |
| ATOM | 722 | O | MET | A | 164 | 12.851 | −3.766 | 13.969 | 1.00 | 4.40 | O |
| ATOM | 723 | N | ILE | A | 165 | 13.880 | −4.863 | 12.296 | 1.00 | 5.00 | N |
| ATOM | 725 | CA | ILE | A | 165 | 12.671 | −5.558 | 11.875 | 1.00 | 4.95 | C |
| ATOM | 727 | CB | ILE | A | 165 | 12.608 | −5.633 | 10.341 | 1.00 | 5.00 | C |
| ATOM | 729 | CG1 | ILE | A | 165 | 12.742 | −4.223 | 9.744 | 1.00 | 4.62 | C |
| ATOM | 732 | CD1 | ILE | A | 165 | 11.748 | −3.208 | 10.282 | 1.00 | 4.68 | C |
| ATOM | 736 | CG2 | ILE | A | 165 | 11.329 | −6.307 | 9.929 | 1.00 | 5.38 | C |
| ATOM | 740 | C | ILE | A | 165 | 12.592 | −6.946 | 12.525 | 1.00 | 5.29 | C |
| ATOM | 741 | O | ILE | A | 165 | 11.558 | −7.315 | 13.093 | 1.00 | 4.78 | O |
| ATOM | 742 | N | GLU | A | 166 | 13.686 | −7.705 | 12.446 | 1.00 | 6.00 | N |
| ATOM | 744 | CA | GLU | A | 166 | 13.785 | −9.017 | 13.085 | 1.00 | 6.87 | C |
| ATOM | 746 | CB | GLU | A | 166 | 13.403 | −10.149 | 12.137 | 1.00 | 7.63 | C |
| ATOM | 749 | CG | GLU | A | 166 | 13.470 | −11.533 | 12.779 | 1.00 | 9.02 | C |
| ATOM | 752 | CD | GLU | A | 166 | 13.503 | −12.667 | 11.766 | 1.00 | 12.97 | C |
| ATOM | 753 | OE1 | GLU | A | 166 | 13.377 | −12.407 | 10.552 | 1.00 | 15.93 | C |
| ATOM | 754 | OE2 | GLU | A | 166 | 13.626 | −13.836 | 12.185 | 1.00 | 14.80 | [symbol] |
| ATOM | 755 | C | GLU | A | 166 | 15.217 | −9.261 | 13.538 | 1.00 | 6.98 | [symbol] |
| ATOM | 756 | O | GLU | A | 166 | 16.045 | −9.709 | 12.749 | 1.00 | 6.92 | [symbol] |
| ATOM | 757 | N | GLY | A | 167 | 15.508 | −8.980 | 14.803 | 1.00 | 7.29 | [symbol] |
| ATOM | 759 | CA | GLY | A | 167 | 16.842 | −9.203 | 15.325 | 1.00 | 7.20 | [symbol] |
| ATOM | 762 | C | GLY | A | 167 | 16.852 | −10.404 | 16.256 | 1.00 | 7.48 | [symbol] |
| ATOM | 763 | O | GLY | A | 167 | 16.056 | −10.456 | 17.191 | 1.00 | 7.66 | [symbol] |
| ATOM | 764 | N | LYS | A | 168 | 17.728 | −11.373 | 15.984 | 1.00 | 7.23 | [symbol] |
| ATOM | 766 | CA | LYS | A | 168 | 17.818 | −12.587 | 16.781 | 1.00 | 7.09 | [symbol] |
| ATOM | 768 | CB | LYS | A | 168 | 18.026 | −13.814 | 15.888 | 1.00 | 7.16 | [symbol] |
| ATOM | 771 | CG | LYS | A | 168 | 16.896 | −14.045 | 14.883 | 1.00 | 8.66 | [symbol] |
| ATOM | 774 | CD | LYS | A | 168 | 17.128 | −15.294 | 14.041 | 1.00 | 10.94 | [symbol] |
| ATOM | 777 | CE | LYS | A | 168 | 16.025 | −15.488 | 13.017 | 1.00 | 12.79 | [symbol] |
| ATOM | 780 | NZ | LYS | A | 168 | 16.093 | −16.800 | 12.293 | 1.00 | 15.02 | [symbol] |
| ATOM | 784 | C | LYS | A | 168 | 18.996 | −12.428 | 17.710 | 1.00 | 6.71 | [symbol] |
| ATOM | 785 | O | LYS | A | 168 | 19.983 | −11.812 | 17.337 | 1.00 | 6.28 | [symbol] |
| ATOM | 786 | N | LEU | A | 169 | 18.878 | −12.976 | 18.916 | 1.00 | 6.73 | [symbol] |
| ATOM | 788 | CA | LEU | A | 169 | 19.933 | −12.900 | 19.902 | 1.00 | 6.40 | [symbol] |
| ATOM | 790 | CB | LEU | A | 169 | 19.498 | −13.607 | 21.186 | 1.00 | 7.01 | [symbol] |
| ATOM | 793 | CG | LEU | A | 169 | 20.474 | −13.428 | 22.350 | 1.00 | 8.27 | [symbol] |
| ATOM | 795 | CD1 | LEU | A | 169 | 20.640 | −11.955 | 22.716 | 1.00 | 9.19 | [symbol] |
| ATOM | 799 | CD2 | LEU | A | 169 | 20.033 | −14.232 | 23.569 | 1.00 | 9.49 | [symbol] |
| ATOM | 803 | C | LEU | A | 169 | 21.217 | −13.531 | 19.363 | 1.00 | 5.72 | [symbol] |
| ATOM | 804 | O | LEU | A | 169 | 21.198 | −14.661 | 18.882 | 1.00 | 5.15 | [symbol] |
| ATOM | 805 | N | ALA | A | 170 | 22.333 | −12.815 | 19.490 | 1.00 | 5.12 | [symbol] |
| ATOM | 807 | CA | ALA | A | 170 | 23.604 | −13.270 | 18.952 | 1.00 | 4.79 | [symbol] |
| ATOM | 809 | CB | ALA | A | 170 | 23.813 | −12.705 | 17.565 | 1.00 | 4.78 | [symbol] |
| ATOM | 813 | C | ALA | A | 170 | 24.731 | −12.807 | 19.849 | 1.00 | 4.58 | [symbol] |
| ATOM | 814 | O | ALA | A | 170 | 24.512 | −12.012 | 20.759 | 1.00 | 4.70 | [symbol] |
| ATOM | 815 | N | THR | A | 171 | 25.933 | −13.309 | 19.581 | 1.00 | 4.52 | [symbol] |
| ATOM | 817 | CA | THR | A | 171 | 27.131 | −12.873 | 20.290 | 1.00 | 4.42 | [symbol] |
| ATOM | 819 | CB | THR | A | 171 | 27.756 | −14.026 | 21.085 | 1.00 | 4.30 | [symbol] |
| ATOM | 821 | OG1 | THR | A | 171 | 26.837 | −14.497 | 22.083 | 1.00 | 4.54 | [symbol] |
| ATOM | 823 | CG2 | THR | A | 171 | 28.977 | −13.555 | 21.899 | 1.00 | 3.98 | [symbol] |
| ATOM | 827 | C | THR | A | 171 | 28.120 | −12.395 | 19.236 | 1.00 | 4.51 | [symbol] |
| ATOM | 828 | O | THR | A | 171 | 28.397 | −13.116 | 18.278 | 1.00 | 4.13 | [symbol] |
| ATOM | 829 | N | LEU | A | 172 | 28.636 | −11.184 | 19.406 | 1.00 | 5.15 | [symbol] |
| ATOM | 831 | CA | LEU | A | 172 | 29.680 | −10.673 | 18.541 | 1.00 | 5.83 | [symbol] |
| ATOM | 833 | CB | LEU | A | 172 | 29.805 | −9.159 | 18.697 | 1.00 | 6.13 | [symbol] |
| ATOM | 836 | CG | LEU | A | 172 | 31.000 | −8.525 | 17.977 | 1.00 | 6.72 | [symbol] |
| ATOM | 838 | CD1 | LEU | A | 172 | 30.994 | −8.877 | 16.496 | 1.00 | 7.77 | [symbol] |
| ATOM | 842 | CD2 | LEU | A | 172 | 30.991 | −7.020 | 18.167 | 1.00 | 7.70 | [symbol] |
| ATOM | 846 | C | LEU | A | 172 | 30.973 | −11.365 | 18.989 | 1.00 | 6.16 | [symbol] |
| ATOM | 847 | O | LEU | A | 172 | 31.430 | −11.155 | 20.111 | 1.00 | 6.30 | [symbol] |
| ATOM | 848 | N | MET | A | 173 | 31.553 | −12.201 | 18.136 | 1.00 | 6.57 | [symbol] |
| ATOM | 850 | CA | MET | A | 173 | 32.750 | −12.965 | 18.519 | 1.00 | 7.57 | [symbol] |
| ATOM | 852 | CB | MET | A | 173 | 32.727 | −14.331 | 17.850 | 1.00 | 7.41 | [symbol] |
| ATOM | 855 | CG | MET | A | 173 | 31.674 | −15.243 | 18.433 | 1.00 | 7.25 | [symbol] |
| ATOM | 858 | SD | MET | A | 173 | 31.804 | −16.910 | 17.822 | 1.00 | 7.63 | [symbol] |
| ATOM | 859 | CE | MET | A | 173 | 31.392 | −16.676 | 16.148 | 1.00 | 8.74 | [symbol] |
| ATOM | 863 | C | MET | A | 173 | 34.060 | −12.277 | 18.188 | 1.00 | 8.46 | [symbol] |
| ATOM | 864 | O | MET | A | 173 | 34.958 | −12.176 | 19.035 | 1.00 | 9.38 | [symbol] |
| ATOM | 865 | N | SER | A | 174 | 34.182 | −11.841 | 16.943 | 1.00 | 9.51 | [symbol] |
| ATOM | 867 | CA | SER | A | 174 | 35.395 | −11.198 | 16.472 | 1.00 | 9.87 | [symbol] |
| ATOM | 869 | CB | SER | A | 174 | 36.388 | −12.243 | 15.965 | 1.00 | 10.03 | [symbol] |
| ATOM | 872 | OG | SER | A | 174 | 35.897 | −12.880 | 14.801 | 1.00 | 10.72 | [symbol] |
| ATOM | 874 | C | SER | A | 174 | 35.028 | −10.268 | 15.331 | 1.00 | 10.19 | [symbol] |
| ATOM | 875 | O | SER | A | 174 | 33.887 | −10.262 | 14.863 | 1.00 | 9.74 | [symbol] |
| ATOM | 876 | N | THR | A | 175 | 36.002 | −9.477 | 14.899 | 1.00 | 10.64 | [symbol] |
| ATOM | 878 | CA | THR | A | 175 | 35.841 | −8.599 | 13.752 | 1.00 | 11.10 | [symbol] |
| ATOM | 880 | CB | THR | A | 175 | 35.735 | −7.125 | 14.184 | 1.00 | 11.03 | [symbol] |
| ATOM | 882 | OG1 | THR | A | 175 | 34.539 | −6.915 | 14.949 | 1.00 | 10.19 | [symbol] |
| ATOM | 884 | CG2 | THR | A | 175 | 35.544 | −6.209 | 12.996 | 1.00 | 11.00 | [symbol] |
| ATOM | 888 | C | THR | A | 175 | 37.079 | −8.797 | 12.894 | 1.00 | 11.71 | [symbol] |
| ATOM | 889 | O | THR | A | 175 | 38.192 | −8.951 | 13.420 | 1.00 | 11.93 | [symbol] |
| ATOM | 890 | N | GLU | A | 176 | 36.884 | −8.814 | 11.584 | 1.00 | 12.23 | [symbol] |
| ATOM | 892 | CA | GLU | A | 176 | 37.970 | −8.970 | 10.629 | 1.00 | 12.70 | [symbol] |
| ATOM | 894 | CB | GLU | A | 176 | 37.824 | −10.291 | 9.873 | 1.00 | 13.24 | [symbol] |
| ATOM | 897 | CG | GLU | A | 176 | 38.742 | −11.388 | 10.361 | 1.00 | 14.72 | [symbol] |
| ATOM | 900 | CD | GLU | A | 176 | 40.202 | −11.034 | 10.166 | 1.00 | 16.74 | [symbol] |
| ATOM | 901 | OE1 | GLU | A | 176 | 40.678 | −11.080 | 9.006 | 1.00 | 18.24 | [symbol] |
| ATOM | 902 | OE2 | GLU | A | 176 | 40.868 | −10.699 | 11.167 | 1.00 | 17.70 | [symbol] |
| ATOM | 903 | C | GLU | A | 176 | 37.920 | −7.818 | 9.635 | 1.00 | 12.62 | [symbol] |
| ATOM | 904 | O | GLU | A | 176 | 36.996 | −7.741 | 8.824 | 1.00 | 12.20 | [symbol] |
| ATOM | 905 | N | GLU | A | 177 | 38.905 | −6.924 | 9.702 | 1.00 | 12.51 | [symbol] |
| ATOM | 907 | CA | GLU | A | 177 | 38.963 | −5.770 | 8.805 | 1.00 | 12.61 | [symbol] |
| ATOM | 909 | CB | GLU | A | 177 | 39.451 | −6.189 | 7.408 | 1.00 | 12.77 | [symbol] |
| ATOM | 912 | CG | GLU | A | 177 | 40.878 | −6.739 | 7.442 | 1.00 | 13.92 | [symbol] |
| ATOM | 915 | CD | GLU | A | 177 | 41.661 | −6.565 | 6.147 | 1.00 | 15.14 | [symbol] |
| ATOM | 916 | OE1 | GLU | A | 177 | 41.102 | −6.076 | 5.141 | 1.00 | 15.39 | [symbol] |
| ATOM | 917 | OE2 | GLU | A | 177 | 42.865 | −6.919 | 6.154 | 1.00 | 15.99 | [symbol] |
| ATOM | 918 | C | GLU | A | 177 | 37.611 | −5.035 | 8.763 | 1.00 | 12.30 | [symbol] |
| ATOM | 919 | O | GLU | A | 177 | 37.070 | −4.742 | 7.689 | 1.00 | 12.20 | [symbol] |
| ATOM | 920 | N | GLY | A | 178 | 37.061 | −4.764 | 9.948 | 1.00 | 11.92 | [symbol] |
| ATOM | 922 | CA | GLY | A | 178 | 35.806 | −4.044 | 10.081 | 1.00 | 11.82 | [symbol] |
| ATOM | 925 | C | GLY | A | 178 | 34.565 | −4.927 | 10.041 | 1.00 | 11.59 | [symbol] |
| ATOM | 926 | O | GLY | A | 178 | 33.496 | −4.517 | 10.489 | 1.00 | 11.30 | [symbol] |
| ATOM | 927 | N | ARG | A | 179 | 34.710 | −6.143 | 9.526 | 1.00 | 11.63 | [symbol] |
| ATOM | 929 | CA | ARG | A | 179 | 33.580 | −7.061 | 9.384 | 1.00 | 11.85 | [symbol] |
| ATOM | 931 | CB | ARG | A | 179 | 33.790 | −7.952 | 8.166 | 1.00 | 12.01 | [symbol] |
| ATOM | 934 | CG | ARG | A | 179 | 34.275 | −7.169 | 6.958 | 1.00 | 13.17 | [symbol] |
| ATOM | 937 | CD | ARG | A | 179 | 34.204 | −7.922 | 5.656 | 1.00 | 14.87 | [symbol] |
| ATOM | 940 | NE | ARG | A | 179 | 34.631 | −7.092 | 4.533 | 1.00 | 16.33 | [symbol] |
| ATOM | 942 | CZ | ARG | A | 179 | 34.270 | −7.297 | 3.276 | 1.00 | 18.07 | [symbol] |
| ATOM | 943 | NH1 | ARG | A | 179 | 33.464 | −8.308 | 2.968 | 1.00 | 18.99 | [symbol] |
| ATOM | 946 | NH2 | ARG | A | 179 | 34.708 | −6.485 | 2.318 | 1.00 | 18.61 | [symbol] |
| ATOM | 949 | C | ARG | A | 179 | 33.403 | −7.917 | 10.632 | 1.00 | 11.75 | [symbol] |
| ATOM | 950 | O | ARG | A | 179 | 34.311 | −8.634 | 11.031 | 1.00 | 11.24 | [symbol] |
| ATOM | 951 | N | PRO | A | 180 | 32.221 | −7.865 | 11.232 | 1.00 | 11.83 | [symbol] |
| ATOM | 952 | CA | PRO | A | 180 | 31.961 | −8.617 | 12.453 | 1.00 | 11.75 | [symbol] |
| ATOM | 954 | CB | PRO | A | 180 | 30.799 | −7.855 | 13.092 | 1.00 | 11.88 | [symbol] |
| ATOM | 957 | CG | PRO | A | 180 | 30.346 | −6.837 | 12.066 | 1.00 | 12.63 | [symbol] |
| ATOM | 960 | CD | PRO | A | 180 | 31.036 | −7.111 | 10.800 | 1.00 | 11.88 | [symbol] |
| ATOM | 963 | C | PRO | A | 180 | 31.523 | −10.035 | 12.171 | 1.00 | 11.47 | [symbol] |
| ATOM | 964 | O | PRO | A | 180 | 30.967 | −10.320 | 11.112 | 1.00 | 11.35 | [symbol] |
| ATOM | 965 | N | HIS | A | 181 | 31.752 | −10.908 | 13.140 | 1.00 | 10.99 | [symbol] |
| ATOM | 967 | CA | HIS | A | 181 | 31.373 | −12.301 | 13.021 | 1.00 | 10.98 | [symbol] |
| ATOM | 969 | CB | HIS | A | 181 | 32.644 | −13.124 | 12.855 | 1.00 | 11.41 | [symbol] |
| ATOM | 972 | CG | HIS | A | 181 | 33.479 | −12.601 | 11.733 | 1.00 | 12.68 | [symbol] |
| ATOM | 973 | ND1 | HIS | A | 181 | 33.096 | −12.732 | 10.413 | 1.00 | 14.33 | [symbol] |
| ATOM | 975 | CE1 | HIS | A | 181 | 33.967 | −12.106 | 9.642 | 1.00 | 14.59 | [symbol] |
| ATOM | 977 | NE2 | HIS | A | 181 | 34.878 | −11.542 | 10.417 | 1.00 | 14.53 | [symbol] |
| ATOM | 979 | CD2 | HIS | A | 181 | 34.573 | −11.806 | 11.730 | 1.00 | 13.34 | [symbol] |
| ATOM | 981 | C | HIS | A | 181 | 30.538 | −12.655 | 14.237 | 1.00 | 10.32 | [symbol] |
| ATOM | 982 | O | HIS | A | 181 | 30.976 | −12.486 | 15.378 | 1.00 | 10.37 | [symbol] |
| ATOM | 983 | N | PHE | A | 182 | 29.315 | −13.102 | 13.977 | 1.00 | 9.47 | [symbol] |
| ATOM | 985 | CA | PHE | A | 182 | 28.364 | −13.389 | 15.037 | 1.00 | 8.95 | [symbol] |
| ATOM | 987 | CB | PHE | A | 182 | 27.026 | −12.691 | 14.756 | 1.00 | 8.73 | [symbol] |
| ATOM | 990 | CG | PHE | A | 182 | 27.112 | −11.193 | 14.743 | 1.00 | 9.26 | [symbol] |
| ATOM | 991 | CD1 | PHE | A | 182 | 27.259 | −10.506 | 13.551 | 1.00 | 9.46 | [symbol] |
| ATOM | 993 | CE1 | PHE | A | 182 | 27.338 | −9.131 | 13.540 | 1.00 | 10.19 | [symbol] |
| ATOM | 995 | CZ | PHE | A | 182 | 27.284 | −8.423 | 14.722 | 1.00 | 11.01 | [symbol] |
| ATOM | 997 | CE2 | PHE | A | 182 | 27.142 | −9.092 | 15.921 | 1.00 | 10.72 | [symbol] |
| ATOM | 999 | CD2 | PHE | A | 182 | 27.053 | −10.472 | 15.926 | 1.00 | 10.44 | [symbol] |
| ATOM | 1001 | C | PHE | A | 182 | 28.094 | −14.868 | 15.172 | 1.00 | 8.41 | [symbol] |
| ATOM | 1002 | O | PHE | A | 182 | 28.181 | −15.608 | 14.200 | 1.00 | 7.90 | [symbol] |
| ATOM | 1003 | N | GLU | A | 183 | 27.779 | −15.294 | 16.390 | 1.00 | 7.69 | [symbol] |
| ATOM | 1005 | CA | GLU | A | 183 | 27.266 | −16.632 | 16.599 | 1.00 | 7.89 | [symbol] |
| ATOM | 1007 | CB | GLU | A | 183 | 28.026 | −17.388 | 17.694 | 1.00 | 8.44 | [symbol] |
| ATOM | 1010 | CG | GLU | A | 183 | 27.883 | −16.811 | 19.074 | 1.00 | 9.36 | [symbol] |
| ATOM | 1013 | CD | GLU | A | 183 | 28.444 | −17.736 | 20.144 | 1.00 | 9.99 | [symbol] |
| ATOM | 1014 | OE1 | GLU | A | 183 | 29.224 | −18.651 | 19.813 | 1.00 | 11.50 | [symbol] |
| ATOM | 1015 | OE2 | GLU | A | 183 | 28.089 | −17.561 | 21.314 | 1.00 | 10.33 | [symbol] |
| ATOM | 1016 | C | GLU | A | 183 | 25.798 | −16.403 | 16.965 | 1.00 | 7.71 | [symbol] |
| ATOM | 1017 | O | GLU | A | 183 | 25.499 | −15.538 | 17.783 | 1.00 | 6.70 | [symbol] |
| ATOM | 1018 | N | LEU | A | 184 | 24.882 | −17.106 | 16.300 | 1.00 | 7.86 | [symbol] |
| ATOM | 1020 | CA | LEU | A | 184 | 23.455 | −16.996 | 16.617 | 1.00 | 8.53 | [symbol] |
| ATOM | 1022 | CB | LEU | A | 184 | 22.575 | −17.474 | 15.455 | 1.00 | 8.98 | [symbol] |
| ATOM | 1025 | CG | LEU | A | 184 | 21.860 | −16.417 | 14.596 | 1.00 | 10.69 | [symbol] |
| ATOM | 1027 | CD1 | LEU | A | 184 | 20.916 | −17.109 | 13.631 | 1.00 | 11.63 | [symbol] |
| ATOM | 1031 | CD2 | LEU | A | 184 | 21.077 | −15.394 | 15.406 | 1.00 | 11.85 | [symbol] |
| ATOM | 1035 | C | LEU | A | 184 | 23.182 | −17.842 | 17.847 | 1.00 | 8.40 | [symbol] |
| ATOM | 1036 | O | LEU | A | 184 | 23.577 | −19.002 | 17.892 | 1.00 | 8.35 | [symbol] |
| ATOM | 1037 | N | MET | A | 185 | 22.524 | −17.268 | 18.847 | 1.00 | 8.65 | [symbol] |
| ATOM | 1039 | CA | MET | A | 185 | 22.274 | −17.994 | 20.092 | 1.00 | 9.49 | [symbol] |
| ATOM | 1041 | CB | MET | A | 185 | 22.166 | −17.032 | 21.277 | 1.00 | 9.31 | [symbol] |
| ATOM | 1044 | CG | MET | A | 185 | 23.331 | −16.044 | 21.374 | 1.00 | 9.03 | [symbol] |
| ATOM | 1047 | SD | MET | A | 185 | 24.981 | −16.799 | 21.366 | 1.00 | 8.67 | [symbol] |
| ATOM | 1048 | CE | MET | A | 185 | 24.996 | −17.487 | 22.988 | 1.00 | 8.51 | [symbol] |
| ATOM | 1052 | C | MET | A | 185 | 21.012 | −18.834 | 19.948 | 1.00 | 10.44 | [symbol] |
| ATOM | 1053 | O | MET | A | 185 | 20.171 | −18.555 | 19.085 | 1.00 | 10.33 | [symbol] |
| ATOM | 1054 | N | PRO | A | 186 | 20.881 | −19.871 | 20.767 | 1.00 | 11.73 | [symbol] |
| ATOM | 1055 | CA | PRO | A | 186 | 19.702 | −20.733 | 20.721 | 1.00 | 12.82 | [symbol] |
| ATOM | 1057 | CB | PRO | A | 186 | 20.089 | −21.907 | 21.625 | 1.00 | 12.75 | [symbol] |
| ATOM | 1060 | CG | PRO | A | 186 | 21.528 | −21.764 | 21.891 | 1.00 | 12.45 | [symbol] |
| ATOM | 1063 | CD | PRO | A | 186 | 21.842 | −20.322 | 21.783 | 1.00 | 12.03 | [symbol] |
| ATOM | 1066 | C | PRO | A | 186 | 18.468 | −20.025 | 21.262 | 1.00 | 13.67 | [symbol] |
| ATOM | 1067 | O | PRO | A | 186 | 18.585 | −18.993 | 21.920 | 1.00 | 14.31 | [symbol] |
| ATOM | 1068 | N | GLY | A | 187 | 17.302 | −20.592 | 21.002 | 1.00 | 14.68 | [symbol] |
| ATOM | 1070 | CA | GLY | A | 187 | 16.051 | −19.982 | 21.411 | 1.00 | 15.36 | [symbol] |
| ATOM | 1073 | C | GLY | A | 187 | 15.353 | −19.478 | 20.158 | 1.00 | 15.85 | [symbol] |
| ATOM | 1074 | O | GLY | A | 187 | 15.922 | −19.536 | 19.066 | 1.00 | 16.33 | [symbol] |
| ATOM | 1075 | N | ASN | A | 188 | 14.126 | −18.993 | 20.295 | 1.00 | 16.29 | [symbol] |
| ATOM | 1077 | CA | ASN | A | 188 | 13.382 | −18.491 | 19.142 | 1.00 | 16.60 | [symbol] |
| ATOM | 1079 | CB | ASN | A | 188 | 12.224 | −19.425 | 18.800 | 1.00 | 16.84 | [symbol] |
| ATOM | 1082 | CG | ASN | A | 188 | 12.685 | −20.693 | 18.107 | 1.00 | 17.32 | [symbol] |
| ATOM | 1083 | OD1 | ASN | A | 188 | 12.476 | −20.872 | 16.903 | 1.00 | 17.36 | [symbol] |
| ATOM | 1084 | ND2 | ASN | A | 188 | 13.311 | −21.584 | 18.869 | 1.00 | 17.33 | [symbol] |
| ATOM | 1087 | C | ASN | A | 188 | 12.851 | −17.074 | 19.338 | 1.00 | 16.40 | [symbol] |
| ATOM | 1088 | O | ASN | A | 188 | 12.059 | −16.586 | 18.529 | 1.00 | 16.84 | [symbol] |
| ATOM | 1089 | N | SER | A | 189 | 13.274 | −16.423 | 20.413 | 1.00 | 15.95 | [symbol] |
| ATOM | 1091 | CA | SER | A | 189 | 12.832 | −15.063 | 20.697 | 1.00 | 15.52 | [symbol] |
| ATOM | 1093 | CB | SER | A | 189 | 13.246 | −14.644 | 22.106 | 1.00 | 15.67 | [symbol] |
| ATOM | 1096 | OG | SER | A | 189 | 12.480 | −15.339 | 23.080 | 1.00 | 16.44 | [symbol] |
| ATOM | 1098 | C | SER | A | 189 | 13.394 | −14.078 | 19.677 | 1.00 | 14.61 | [symbol] |
| ATOM | 1099 | O | SER | A | 189 | 14.597 | −13.968 | 19.484 | 1.00 | 15.04 | [symbol] |
| ATOM | 1100 | N | VAL | A | 190 | 12.501 | −13.380 | 18.998 | 1.00 | 13.84 | [symbol] |
| ATOM | 1102 | CA | VAL | A | 190 | 12.926 | −12.357 | 18.061 | 1.00 | 12.46 | [symbol] |
| ATOM | 1104 | CB | VAL | A | 190 | 12.127 | −12.417 | 16.747 | 1.00 | 12.81 | [symbol] |
| ATOM | 1106 | CG1 | VAL | A | 190 | 12.524 | −13.627 | 15.919 | 1.00 | 13.83 | [symbol] |
| ATOM | 1110 | CG2 | VAL | A | 190 | 10.629 | −12.440 | 17.024 | 1.00 | 13.36 | [symbol] |
| ATOM | 1114 | C | VAL | A | 190 | 12.671 | −11.017 | 18.737 | 1.00 | 10.30 | [symbol] |
| ATOM | 1115 | O | VAL | A | 190 | 11.856 | −10.935 | 19.651 | 1.00 | 9.90 | [symbol] |
| ATOM | 1116 | N | TYR | A | 191 | 13.365 | −9.978 | 18.297 | 1.00 | 7.89 | [symbol] |
| ATOM | 1118 | CA | TYR | A | 191 | 13.065 | −8.632 | 18.748 | 1.00 | 6.57 | [symbol] |
| ATOM | 1120 | CB | TYR | A | 191 | 14.196 | −8.097 | 19.599 | 1.00 | 6.34 | [symbol] |
| ATOM | 1123 | CG | TYR | A | 191 | 14.589 | −9.048 | 20.680 | 1.00 | 5.06 | [symbol] |
| ATOM | 1124 | CD1 | TYR | A | 191 | 15.581 | −9.990 | 20.460 | 1.00 | 5.67 | [symbol] |
| ATOM | 1126 | CE1 | TYR | A | 191 | 15.982 | −10.870 | 21.483 | 1.00 | 4.58 | [symbol] |
| ATOM | 1128 | CZ | TYR | A | 191 | 15.358 | −10.831 | 22.705 | 1.00 | 3.98 | [symbol] |
| ATOM | 1129 | OH | TYR | A | 191 | 15.770 | −11.722 | 23.688 | 1.00 | 5.42 | [symbol] |
| ATOM | 1131 | CE2 | TYR | A | 191 | 14.374 | −9.904 | 22.956 | 1.00 | 3.96 | [symbol] |
| ATOM | 1133 | CD2 | TYR | A | 191 | 13.976 | −9.015 | 21.936 | 1.00 | 4.68 | [symbol] |
| ATOM | 1135 | C | TYR | A | 191 | 12.867 | −7.717 | 17.548 | 1.00 | 5.86 | [symbol] |
| ATOM | 1136 | O | TYR | A | 191 | 13.442 | −7.941 | 16.490 | 1.00 | 5.27 | [symbol] |
| ATOM | 1137 | N | HIS | A | 192 | 12.064 | −6.680 | 17.733 | 1.00 | 5.52 | [symbol] |
| ATOM | 1139 | CA | HIS | A | 192 | 11.834 | −5.683 | 16.695 | 1.00 | 5.42 | [symbol] |
| ATOM | 1141 | CB | HIS | A | 192 | 10.640 | −6.082 | 15.840 | 1.00 | 5.81 | [symbol] |
| ATOM | 1144 | CG | HIS | A | 192 | 9.337 | −6.131 | 16.581 | 1.00 | 7.52 | [symbol] |
| ATOM | 1145 | ND1 | HIS | A | 192 | 8.968 | −7.201 | 17.368 | 1.00 | 8.54 | [symbol] |
| ATOM | 1147 | CE1 | HIS | A | 192 | 7.767 | −6.981 | 17.874 | 1.00 | 8.22 | [symbol] |
| ATOM | 1149 | NE2 | HIS | A | 192 | 7.340 | −5.810 | 17.439 | 1.00 | 8.91 | [symbol] |
| ATOM | 1151 | CD2 | HIS | A | 192 | 8.297 | −5.263 | 16.618 | 1.00 | 8.65 | [symbol] |
| ATOM | 1153 | C | HIS | A | 192 | 11.604 | −4.308 | 17.310 | 1.00 | 4.59 | [symbol] |
| ATOM | 1154 | O | HIS | A | 192 | 11.297 | −4.196 | 18.500 | 1.00 | 4.80 | [symbol] |
| ATOM | 1155 | N | PHE | A | 193 | 11.814 | −3.269 | 16.514 | 1.00 | 3.78 | [symbol] |
| ATOM | 1157 | CA | PHE | A | 193 | 11.499 | −1.910 | 16.920 | 1.00 | 4.00 | [symbol] |
| ATOM | 1159 | CB | PHE | A | 193 | 11.713 | −0.937 | 15.762 | 1.00 | 3.98 | [symbol] |
| ATOM | 1162 | CG | PHE | A | 193 | 11.543 | 0.513 | 16.132 | 1.00 | 4.65 | [symbol] |
| ATOM | 1163 | CD1 | PHE | A | 193 | 12.275 | 1.085 | 17.162 | 1.00 | 5.34 | [symbol] |
| ATOM | 1165 | CE1 | PHE | A | 193 | 12.114 | 2.427 | 17.484 | 1.00 | 5.11 | [symbol] |
| ATOM | 1167 | CZ | PHE | A | 193 | 11.224 | 3.201 | 16.773 | 1.00 | 6.20 | [symbol] |
| ATOM | 1169 | CE2 | PHE | A | 193 | 10.492 | 2.641 | 15.759 | 1.00 | 6.35 | [symbol] |
| ATOM | 1171 | CD2 | PHE | A | 193 | 10.654 | 1.307 | 15.440 | 1.00 | 6.00 | [symbol] |
| ATOM | 1173 | C | PHE | A | 193 | 10.043 | −1.917 | 17.354 | 1.00 | 4.11 | [symbol] |
| ATOM | 1174 | O | PHE | A | 193 | 9.153 | −2.237 | 16.552 | 1.00 | 3.97 | [symbol] |
| ATOM | 1175 | N | ASP | A | 194 | 9.794 | −1.561 | 18.607 | 1.00 | 4.35 | [symbol] |
| ATOM | 1177 | CA | ASP | A | 194 | 8.458 | −1.645 | 19.176 | 1.00 | 4.97 | [symbol] |
| ATOM | 1179 | CB | ASP | A | 194 | 8.290 | −3.008 | 19.861 | 1.00 | 5.31 | [symbol] |
| ATOM | 1182 | CG | ASP | A | 194 | 6.876 | −3.264 | 20.360 | 1.00 | 4.89 | [symbol] |
| ATOM | 1183 | OD1 | ASP | A | 194 | 6.731 | −3.777 | 21.498 | 1.00 | 4.65 | [symbol] |
| ATOM | 1184 | OD2 | ASP | A | 194 | 5.855 | −2.991 | 19.698 | 1.00 | 6.48 | [symbol] |
| ATOM | 1185 | C | ASP | A | 194 | 8.249 | −0.494 | 20.162 | 1.00 | 5.45 | [symbol] |
| ATOM | 1186 | O | ASP | A | 194 | 8.101 | −0.711 | 21.369 | 1.00 | 5.31 | [symbol] |
| ATOM | 1187 | N | LYS | A | 195 | 8.246 | 0.728 | 19.634 | 1.00 | 5.95 | [symbol] |
| ATOM | 1189 | CA | LYS | A | 195 | 8.067 | 1.932 | 20.444 | 1.00 | 6.42 | [symbol] |
| ATOM | 1191 | CB | LYS | A | 195 | 8.163 | 3.173 | 19.549 | 1.00 | 6.85 | [symbol] |
| ATOM | 1194 | CG | LYS | A | 195 | 8.499 | 4.463 | 20.274 | 1.00 | 7.53 | [symbol] |
| ATOM | 1197 | CD | LYS | A | 195 | 8.571 | 5.668 | 19.330 | 1.00 | 9.24 | [symbol] |
| ATOM | 1200 | CE | LYS | A | 195 | 8.671 | 6.977 | 20.117 | 1.00 | 10.07 | [symbol] |
| ATOM | 1203 | NZ | LYS | A | 195 | 8.535 | 8.203 | 19.303 | 1.00 | 10.82 | [symbol] |
| ATOM | 1207 | C | LYS | A | 195 | 6.718 | 1.897 | 21.167 | 1.00 | 6.73 | [symbol] |
| ATOM | 1208 | O | LYS | A | 195 | 5.701 | 1.507 | 20.582 | 1.00 | 5.99 | [symbol] |
| ATOM | 1209 | N | SER | A | 196 | 6.697 | 2.294 | 22.438 | 1.00 | 6.97 | [symbol] |
| ATOM | 1211 | CA | SER | A | 196 | 5.443 | 2.313 | 23.210 | 1.00 | 7.85 | [symbol] |
| ATOM | 1213 | CB | SER | A | 196 | 5.741 | 2.699 | 24.653 | 1.00 | 7.66 | [symbol] |
| ATOM | 1216 | OG | SER | A | 196 | 6.188 | 4.036 | 24.712 | 1.00 | 7.51 | [symbol] |
| ATOM | 1218 | C | SER | A | 196 | 4.375 | 3.286 | 22.654 | 1.00 | 8.71 | [symbol] |
| ATOM | 1219 | O | SER | A | 196 | 4.719 | 4.256 | 21.987 | 1.00 | 8.40 | [symbol] |
| ATOM | 1220 | N | THR | A | 197 | 3.100 | 3.017 | 22.971 | 1.00 | 10.30 | [symbol] |
| ATOM | 1222 | CA | THR | A | 197 | 1.936 | 3.843 | 22.568 | 1.00 | 11.43 | [symbol] |
| ATOM | 1224 | CB | THR | A | 197 | 1.071 | 3.161 | 21.458 | 1.00 | 11.80 | [symbol] |
| ATOM | 1226 | OG1 | THR | A | 197 | 0.256 | 2.116 | 22.010 | 1.00 | 12.85 | [symbol] |
| ATOM | 1228 | CG2 | THR | A | 197 | 1.933 | 2.464 | 20.416 | 1.00 | 11.59 | [symbol] |
| ATOM | 1232 | C | THR | A | 197 | 1.075 | 4.129 | 23.811 | 1.00 | 12.45 | [symbol] |
| ATOM | 1233 | O | THR | A | 197 | 1.409 | 3.702 | 24.907 | 1.00 | 12.65 | [symbol] |
| ATOM | 1234 | N | SER | A | 198 | −0.051 | 4.818 | 23.658 | 1.00 | 13.61 | [symbol] |
| ATOM | 1236 | CA | SER | A | 198 | −0.866 | 5.162 | 24.829 | 1.00 | 14.55 | [symbol] |
| ATOM | 1238 | CB | SER | A | 198 | −1.735 | 6.376 | 24.549 | 1.00 | 14.51 | [symbol] |
| ATOM | 1241 | OG | SER | A | 198 | −2.410 | 6.197 | 23.325 | 1.00 | 13.76 | [symbol] |
| ATOM | 1243 | C | SER | A | 198 | −1.754 | 3.982 | 25.168 | 1.00 | 15.46 | [symbol] |
| ATOM | 1244 | O | SER | A | 198 | −2.209 | 3.819 | 26.297 | 1.00 | 16.73 | [symbol] |
| ATOM | 1245 | N | SER | A | 199 | −2.002 | 3.166 | 24.158 | 1.00 | 15.77 | [symbol] |
| ATOM | 1247 | CA | SER | A | 199 | −2.797 | 1.972 | 24.318 | 1.00 | 16.24 | [symbol] |
| ATOM | 1249 | CB | SER | A | 199 | −3.745 | 1.838 | 23.130 | 1.00 | 16.54 | [symbol] |
| ATOM | 1252 | OG | SER | A | 199 | −3.053 | 1.414 | 21.970 | 1.00 | 17.04 | [symbol] |
| ATOM | 1254 | C | SER | A | 199 | −1.869 | 0.749 | 24.425 | 1.00 | 16.21 | [symbol] |
| ATOM | 1255 | O | SER | A | 199 | −2.332 | −0.385 | 24.516 | 1.00 | 16.42 | [symbol] |
| ATOM | 1256 | N | CYS | A | 200 | −0.559 | 0.976 | 24.438 | 1.00 | 15.86 | [symbol] |
| ATOM | 1258 | CA | CYS | A | 200 | 0.386 | −0.137 | 24.428 | 1.00 | 15.68 | [symbol] |
| ATOM | 1260 | CB | CYS | A | 200 | 0.453 | −0.723 | 23.012 | 1.00 | 15.84 | [symbol] |
| ATOM | 1263 | SG | CYS | A | 200 | 1.110 | −2.412 | 22.884 | 1.00 | 17.21 | [symbol] |
| ATOM | 1264 | C | CYS | A | 200 | 1.799 | 0.229 | 24.868 | 1.00 | 14.77 | [symbol] |
| ATOM | 1265 | O | CYS | A | 200 | 2.528 | 0.896 | 24.140 | 1.00 | 15.85 | [symbol] |
| ATOM | 1266 | N | ILE | A | 201 | 2.199 | −0.229 | 26.052 | 1.00 | 13.28 | [symbol] |
| ATOM | 1268 | CA | ILE | A | 201 | 3.565 | −0.032 | 26.518 | 1.00 | 11.96 | [symbol] |
| ATOM | 1270 | CB | ILE | A | 201 | 3.689 | −0.385 | 28.025 | 1.00 | 12.01 | [symbol] |
| ATOM | 1272 | CG1 | ILE | A | 201 | 5.108 | −0.137 | 28.534 | 1.00 | 12.80 | [symbol] |
| ATOM | 1275 | CD1 | ILE | A | 201 | 5.162 | 0.134 | 30.028 | 1.00 | 13.95 | [symbol] |
| ATOM | 1279 | CG2 | ILE | A | 201 | 3.284 | −1.817 | 28.271 | 1.00 | 11.38 | [symbol] |
| ATOM | 1283 | C | ILE | A | 201 | 4.519 | −0.889 | 25.680 | 1.00 | 10.34 | [symbol] |
| ATOM | 1284 | O | ILE | A | 201 | 5.630 | −0.469 | 25.380 | 1.00 | 9.68 | [symbol] |
| ATOM | 1285 | N | SER | A | 202 | 4.057 | −2.075 | 25.284 | 1.00 | 9.04 | [symbol] |
| ATOM | 1287 | CA | SER | A | 202 | 4.859 | −3.027 | 24.519 | 1.00 | 8.50 | [symbol] |
| ATOM | 1289 | CB | SER | A | 202 | 5.929 | −3.672 | 25.431 | 1.00 | 8.52 | [symbol] |
| ATOM | 1292 | OG | SER | A | 202 | 6.646 | −4.704 | 24.774 | 1.00 | 6.90 | [symbol] |
| ATOM | 1294 | C | SER | A | 202 | 3.958 | −4.114 | 23.917 | 1.00 | 8.85 | [symbol] |
| ATOM | 1295 | O | SER | A | 202 | 2.965 | −4.520 | 24.529 | 1.00 | 8.96 | [symbol] |
| ATOM | 1296 | N | THR | A | 203 | 4.282 | −4.557 | 22.703 | 1.00 | 8.53 | [symbol] |
| ATOM | 1298 | CA | THR | A | 203 | 3.553 | −5.638 | 22.063 | 1.00 | 7.99 | [symbol] |
| ATOM | 1300 | CB | THR | A | 203 | 4.000 | −5.799 | 20.604 | 1.00 | 8.51 | [symbol] |
| ATOM | 1302 | OG1 | THR | A | 203 | 3.927 | −4.539 | 19.917 | 1.00 | 9.13 | [symbol] |
| ATOM | 1304 | CG2 | THR | A | 203 | 3.031 | −6.653 | 19.837 | 1.00 | 9.16 | [symbol] |
| ATOM | 1308 | C | THR | A | 203 | 3.802 | −6.953 | 22.780 | 1.00 | 7.35 | [symbol] |
| ATOM | 1309 | O | THR | A | 203 | 2.964 | −7.850 | 22.759 | 1.00 | 6.77 | [symbol] |
| ATOM | 1310 | N | ASN | A | 204 | 4.983 | −7.072 | 23.388 | 1.00 | 6.67 | [symbol] |
| ATOM | 1312 | CA | ASN | A | 204 | 5.358 | −8.269 | 24.108 | 1.00 | 6.32 | [symbol] |
| ATOM | 1314 | CB | ASN | A | 204 | 6.430 | −9.031 | 23.340 | 1.00 | 7.02 | [symbol] |
| ATOM | 1317 | CG | ASN | A | 204 | 6.076 | −9.229 | 21.901 | 1.00 | 9.04 | [symbol] |
| ATOM | 1318 | OD1 | ASN | A | 204 | 5.442 | −10.222 | 21.545 | 1.00 | 12.06 | [symbol] |
| ATOM | 1319 | ND2 | ASN | A | 204 | 6.473 | −8.285 | 21.050 | 1.00 | 10.74 | [symbol] |
| ATOM | 1322 | C | ASN | A | 204 | 5.896 | −7.849 | 25.476 | 1.00 | 5.34 | [symbol] |
| ATOM | 1323 | O | ASN | A | 204 | 7.112 | −7.902 | 25.712 | 1.00 | 4.91 | [symbol] |
| ATOM | 1324 | N | ALA | A | 205 | 4.991 | −7.414 | 26.355 | 1.00 | 4.25 | [symbol] |
| ATOM | 1326 | CA | ALA | A | 205 | 5.353 | −6.895 | 27.687 | 1.00 | 4.45 | [symbol] |
| ATOM | 1328 | CB | ALA | A | 205 | 4.103 | −6.433 | 28.415 | 1.00 | 4.36 | [symbol] |
| ATOM | 1332 | C | ALA | A | 205 | 6.107 | −7.869 | 28.578 | 1.00 | 4.02 | [symbol] |
| ATOM | 1333 | O | ALA | A | 205 | 6.748 | −7.445 | 29.545 | 1.00 | 4.26 | [symbol] |
| ATOM | 1334 | N | LEU | A | 206 | 5.996 | −9.163 | 28.293 | 1.00 | 4.13 | [symbol] |
| ATOM | 1336 | CA | LEU | A | 206 | 6.639 | −10.193 | 29.127 | 1.00 | 4.05 | [symbol] |
| ATOM | 1338 | CB | LEU | A | 206 | 5.651 | −11.310 | 29.467 | 1.00 | 4.39 | [symbol] |
| ATOM | 1341 | CG | LEU | A | 206 | 4.540 | −10.873 | 30.442 | 1.00 | 5.48 | [symbol] |
| ATOM | 1343 | CD1 | LEU | A | 206 | 3.623 | −12.031 | 30.791 | 1.00 | 6.08 | [symbol] |
| ATOM | 1347 | CD2 | LEU | A | 206 | 5.105 | −10.247 | 31.717 | 1.00 | 6.03 | [symbol] |
| ATOM | 1351 | C | LEU | A | 206 | 7.883 | −10.780 | 28.476 | 1.00 | 3.35 | [symbol] |
| ATOM | 1352 | O | LEU | A | 206 | 8.445 | −11.755 | 28.972 | 1.00 | 3.40 | [symbol] |
| ATOM | 1353 | N | LEU | A | 207 | 8.298 | −10.190 | 27.363 | 1.00 | 2.61 | [symbol] |
| ATOM | 1355 | CA | LEU | A | 207 | 9.509 | −10.600 | 26.677 | 1.00 | 2.48 | [symbol] |
| ATOM | 1357 | CB | LEU | A | 207 | 9.370 | −10.388 | 25.167 | 1.00 | 2.30 | [symbol] |
| ATOM | 1360 | CG | LEU | A | 207 | 10.658 | −10.610 | 24.363 | 1.00 | 2.38 | [symbol] |
| ATOM | 1362 | CD1 | LEU | A | 207 | 11.119 | −12.048 | 24.473 | 1.00 | 2.94 | [symbol] |
| ATOM | 1366 | CD2 | LEU | A | 207 | 10.491 | −10.235 | 22.889 | 1.00 | 2.26 | [symbol] |
| ATOM | 1370 | C | LEU | A | 207 | 10.639 | −9.727 | 27.221 | 1.00 | 2.20 | [symbol] |
| ATOM | 1371 | O | LEU | A | 207 | 10.736 | −8.557 | 26.881 | 1.00 | 2.36 | [symbol] |
| ATOM | 1372 | N | PRO | A | 208 | 11.487 | −10.266 | 28.082 | 1.00 | 2.31 | [symbol] |
| ATOM | 1373 | CA | PRO | A | 208 | 12.541 | −9.447 | 28.692 | 1.00 | 2.22 | [symbol] |
| ATOM | 1375 | CB | PRO | A | 208 | 13.158 | −10.383 | 29.746 | 1.00 | 2.19 | [symbol] |
| ATOM | 1378 | CG | PRO | A | 208 | 12.238 | −11.522 | 29.864 | 1.00 | 2.61 | [symbol] |
| ATOM | 1381 | CD | PRO | A | 208 | 11.525 | −11.654 | 28.555 | 1.00 | 2.13 | [symbol] |
| ATOM | 1384 | C | PRO | A | 208 | 13.622 | −9.002 | 27.729 | 1.00 | 2.17 | [symbol] |
| ATOM | 1385 | O | PRO | A | 208 | 13.744 | −9.544 | 26.634 | 1.00 | 2.75 | [symbol] |
| ATOM | 1386 | N | ASP | A | 209 | 14.382 | −7.993 | 28.133 | 1.00 | 2.07 | [symbol] |
| ATOM | 1388 | CA | ASP | A | 209 | 15.537 | −7.558 | 27.370 | 1.00 | 2.07 | [symbol] |
| ATOM | 1390 | CB | ASP | A | 209 | 15.873 | −6.108 | 27.671 | 1.00 | 2.16 | [symbol] |
| ATOM | 1393 | CG | ASP | A | 209 | 17.092 | −5.642 | 26.921 | 1.00 | 2.83 | [symbol] |
| ATOM | 1394 | OD1 | ASP | A | 209 | 18.215 | −5.970 | 27.366 | 1.00 | 2.00 | [symbol] |
| ATOM | 1395 | OD2 | ASP | A | 209 | 17.026 | −4.987 | 25.858 | 1.00 | 3.37 | [symbol] |
| ATOM | 1396 | C | ASP | A | 209 | 16.696 | −8.488 | 27.758 | 1.00 | 2.08 | [symbol] |
| ATOM | 1397 | O | ASP | A | 209 | 16.986 | −8.667 | 28.940 | 1.00 | 2.17 | [symbol] |
| ATOM | 1398 | N | PRO | A | 210 | 17.378 | −9.050 | 26.770 | 1.00 | 2.13 | [symbol] |
| ATOM | 1399 | CA | PRO | A | 210 | 18.411 | −10.064 | 27.006 | 1.00 | 2.49 | [symbol] |
| ATOM | 1401 | CB | PRO | A | 210 | 18.719 | −10.589 | 25.599 | 1.00 | 2.35 | [symbol] |
| ATOM | 1404 | CG | PRO | A | 210 | 18.212 | −9.574 | 24.665 | 1.00 | 2.21 | [symbol] |
| ATOM | 1407 | CD | PRO | A | 210 | 17.258 | −8.704 | 25.352 | 1.00 | 2.18 | [symbol] |
| ATOM | 1410 | C | PRO | A | 210 | 19.684 | −9.538 | 27.651 | 1.00 | 2.93 | [symbol] |
| ATOM | 1411 | O | PRO | A | 210 | 20.321 | −10.299 | 28.361 | 1.00 | 2.79 | [symbol] |
| ATOM | 1412 | N | TYR | A | 211 | 20.043 | −8.283 | 27.398 | 1.00 | 3.18 | [symbol] |
| ATOM | 1414 | CA | TYR | A | 211 | 21.242 | −7.682 | 27.950 | 1.00 | 3.33 | [symbol] |
| ATOM | 1416 | CB | TYR | A | 211 | 21.570 | −6.388 | 27.200 | 1.00 | 3.46 | [symbol] |
| ATOM | 1419 | CG | TYR | A | 211 | 22.666 | −5.539 | 27.817 | 1.00 | 2.30 | [symbol] |
| ATOM | 1420 | CD1 | TYR | A | 211 | 23.995 | −5.687 | 27.428 | 1.00 | 2.58 | [symbol] |
| ATOM | 1422 | CE1 | TYR | A | 211 | 25.015 | −4.900 | 28.010 | 1.00 | 2.00 | [symbol] |
| ATOM | 1424 | CZ | TYR | A | 211 | 24.685 | −3.963 | 28.967 | 1.00 | 2.27 | [symbol] |
| ATOM | 1425 | OH | TYR | A | 211 | 25.673 | −3.168 | 29.545 | 1.00 | 2.08 | [symbol] |
| ATOM | 1427 | CE2 | TYR | A | 211 | 23.356 | −3.791 | 29.346 | 1.00 | 2.01 | [symbol] |
| ATOM | 1429 | CD2 | TYR | A | 211 | 22.371 | −4.583 | 28.785 | 1.00 | 2.29 | [symbol] |
| ATOM | 1431 | C | TYR | A | 211 | 20.962 | −7.403 | 29.419 | 1.00 | 3.70 | [symbol] |
| ATOM | 1432 | O | TYR | A | 211 | 21.770 | −7.707 | 30.297 | 1.00 | 4.00 | [symbol] |
| ATOM | 1433 | N | GLU | A | 212 | 19.782 | −6.845 | 29.670 | 1.00 | 3.95 | [symbol] |
| ATOM | 1435 | CA | GLU | A | 212 | 19.348 | −6.519 | 31.018 | 1.00 | 4.61 | [symbol] |
| ATOM | 1437 | CB | GLU | A | 212 | 18.024 | −5.773 | 30.929 | 1.00 | 4.52 | [symbol] |
| ATOM | 1440 | CG | GLU | A | 212 | 17.515 | −5.155 | 32.202 | 1.00 | 4.94 | [symbol] |
| ATOM | 1443 | CD | GLU | A | 212 | 16.226 | −4.392 | 31.966 | 1.00 | 4.99 | [symbol] |
| ATOM | 1444 | OE1 | GLU | A | 212 | 15.347 | −4.950 | 31.280 | 1.00 | 5.00 | [symbol] |
| ATOM | 1445 | OE2 | GLU | A | 212 | 16.086 | −3.249 | 32.445 | 1.00 | 5.13 | [symbol] |
| ATOM | 1446 | C | GLU | A | 212 | 19.214 | −7.802 | 31.846 | 1.00 | 5.07 | [symbol] |
| ATOM | 1447 | O | GLU | A | 212 | 19.640 | −7.862 | 33.002 | 1.00 | 4.83 | [symbol] |
| ATOM | 1448 | N | SER | A | 213 | 18.638 | −8.830 | 31.228 | 1.00 | 5.54 | [symbol] |
| ATOM | 1450 | CA | SER | A | 213 | 18.421 | −10.135 | 31.850 | 1.00 | 6.29 | [symbol] |
| ATOM | 1452 | CB | SER | A | 213 | 17.821 | −11.080 | 30.797 | 1.00 | 6.89 | [symbol] |
| ATOM | 1455 | OG | SER | A | 213 | 17.892 | −12.428 | 31.216 | 1.00 | 9.71 | [symbol] |
| ATOM | 1457 | C | SER | A | 213 | 19.702 | −10.733 | 32.444 | 1.00 | 6.29 | [symbol] |
| ATOM | 1458 | O | SER | A | 213 | 19.665 | −11.412 | 33.479 | 1.00 | 6.32 | [symbol] |
| ATOM | 1459 | N | GLU | A | 214 | 20.832 | −10.472 | 31.796 | 1.00 | 6.12 | [symbol] |
| ATOM | 1461 | CA | GLU | A | 214 | 22.120 | −10.969 | 32.278 | 1.00 | 5.99 | [symbol] |
| ATOM | 1463 | CB | GLU | A | 214 | 23.164 | −10.915 | 31.166 | 1.00 | 6.73 | [symbol] |
| ATOM | 1466 | CG | GLU | A | 214 | 22.949 | −11.904 | 30.036 | 1.00 | 9.30 | [symbol] |
| ATOM | 1469 | CD | GLU | A | 214 | 24.057 | −11.830 | 28.999 | 1.00 | 12.66 | [symbol] |
| ATOM | 1470 | OE1 | GLU | A | 214 | 24.949 | −10.962 | 29.143 | 1.00 | 14.58 | [symbol] |
| ATOM | 1471 | OE2 | GLU | A | 214 | 24.032 | −12.631 | 28.041 | 1.00 | 14.18 | [symbol] |
| ATOM | 1472 | C | GLU | A | 214 | 22.679 | −10.174 | 33.454 | 1.00 | 5.39 | [symbol] |
| ATOM | 1473 | O | GLU | A | 214 | 23.514 | −10.687 | 34.206 | 1.00 | 4.38 | [symbol] |
| ATOM | 1474 | N | ARG | A | 215 | 22.222 | −8.937 | 33.610 | 1.00 | 4.75 | [symbol] |
| ATOM | 1476 | CA | ARG | A | 215 | 22.830 | −8.018 | 34.578 | 1.00 | 4.90 | [symbol] |
| ATOM | 1478 | CB | ARG | A | 215 | 23.306 | −6.746 | 33.853 | 1.00 | 5.24 | [symbol] |
| ATOM | 1481 | CG | ARG | A | 215 | 24.713 | −6.896 | 33.276 | 1.00 | 8.07 | [symbol] |
| ATOM | 1484 | CD | ARG | A | 215 | 25.145 | −5.883 | 32.213 | 1.00 | 10.65 | [symbol] |
| ATOM | 1487 | NE | ARG | A | 215 | 24.801 | −6.383 | 30.908 | 1.00 | 14.29 | [symbol] |
| ATOM | 1489 | CZ | ARG | A | 215 | 25.577 | −7.088 | 30.089 | 1.00 | 15.26 | [symbol] |
| ATOM | 1490 | NH1 | ARG | A | 215 | 26.820 | −7.388 | 30.367 | 1.00 | 15.54 | [symbol] |
| ATOM | 1493 | NH2 | ARG | A | 215 | 25.065 | −7.491 | 28.943 | 1.00 | 17.12 | [symbol] |
| ATOM | 1496 | C | ARG | A | 215 | 21.957 | −7.671 | 35.791 | 1.00 | 4.12 | [symbol] |
| ATOM | 1497 | O | ARG | A | 215 | 22.487 | −7.282 | 36.806 | 1.00 | 3.33 | [symbol] |
| ATOM | 1498 | N | VAL | A | 216 | 20.629 | −7.807 | 35.709 | 1.00 | 3.09 | [symbol] |
| ATOM | 1500 | CA | VAL | A | 216 | 19.809 | −7.429 | 36.873 | 1.00 | 2.91 | [symbol] |
| ATOM | 1502 | CB | VAL | A | 216 | 19.230 | −6.002 | 36.738 | 1.00 | 2.96 | [symbol] |
| ATOM | 1504 | CG1 | VAL | A | 216 | 20.332 | −4.974 | 36.602 | 1.00 | 3.75 | [symbol] |
| ATOM | 1508 | CG2 | VAL | A | 216 | 18.252 | −5.921 | 35.551 | 1.00 | 2.83 | [symbol] |
| ATOM | 1512 | C | VAL | A | 216 | 18.647 | −8.355 | 37.189 | 1.00 | 2.98 | [symbol] |
| ATOM | 1513 | O | VAL | A | 216 | 18.168 | −9.109 | 36.331 | 1.00 | 2.44 | [symbol] |
| ATOM | 1514 | N | TYR | A | 217 | 18.199 | −8.289 | 38.440 | 1.00 | 2.76 | [symbol] |
| ATOM | 1516 | CA | TYR | A | 217 | 17.009 | −9.020 | 38.865 | 1.00 | 2.74 | [symbol] |
| ATOM | 1518 | CB | TYR | A | 217 | 17.341 | −10.412 | 39.382 | 1.00 | 2.54 | [symbol] |
| ATOM | 1521 | CG | TYR | A | 217 | 18.125 | −10.454 | 40.662 | 1.00 | 2.38 | [symbol] |
| ATOM | 1522 | CD1 | TYR | A | 217 | 17.486 | −10.645 | 41.874 | 1.00 | 2.54 | [symbol] |
| ATOM | 1524 | CE1 | TYR | A | 217 | 18.183 | −10.712 | 43.040 | 1.00 | 2.32 | [symbol] |
| ATOM | 1526 | CZ | TYR | A | 217 | 19.553 | −10.587 | 43.022 | 1.00 | 2.27 | [symbol] |
| ATOM | 1527 | OH | TYR | A | 217 | 20.236 | −10.656 | 44.219 | 1.00 | 2.99 | [symbol] |
| ATOM | 1529 | CE2 | TYR | A | 217 | 20.220 | −10.409 | 41.826 | 1.00 | 2.26 | [symbol] |
| ATOM | 1531 | CD2 | TYR | A | 217 | 19.507 | −10.339 | 40.657 | 1.00 | 2.43 | [symbol] |
| ATOM | 1533 | C | TYR | A | 217 | 16.202 | −8.208 | 39.881 | 1.00 | 2.77 | [symbol] |
| ATOM | 1534 | O | TYR | A | 217 | 16.737 | −7.327 | 40.557 | 1.00 | 3.00 | [symbol] |
| ATOM | 1535 | N | VAL | A | 218 | 14.908 | −8.497 | 39.961 | 1.00 | 2.44 | [symbol] |
| ATOM | 1537 | CA | VAL | A | 218 | 14.014 | −7.831 | 40.890 | 1.00 | 2.69 | [symbol] |
| ATOM | 1539 | CB | VAL | A | 218 | 12.632 | −7.579 | 40.228 | 1.00 | 3.00 | [symbol] |
| ATOM | 1541 | CG1 | VAL | A | 218 | 11.610 | −7.097 | 41.245 | 1.00 | 3.76 | [symbol] |
| ATOM | 1545 | CG2 | VAL | A | 218 | 12.778 | −6.577 | 39.140 | 1.00 | 3.43 | [symbol] |
| ATOM | 1549 | C | VAL | A | 218 | 13.833 | −8.702 | 42.120 | 1.00 | 2.66 | [symbol] |
| ATOM | 1550 | O | VAL | A | 218 | 13.617 | −9.907 | 42.005 | 1.00 | 3.28 | [symbol] |
| ATOM | 1551 | N | ALA | A | 219 | 13.917 | −8.091 | 43.296 | 1.00 | 2.57 | [symbol] |
| ATOM | 1553 | CA | ALA | A | 219 | 13.739 | −8.791 | 44.555 | 1.00 | 2.48 | [symbol] |
| ATOM | 1555 | CB | ALA | A | 219 | 15.054 | −9.468 | 45.000 | 1.00 | 2.68 | [symbol] |
| ATOM | 1559 | C | ALA | A | 219 | 13.301 | −7.758 | 45.585 | 1.00 | 2.60 | [symbol] |
| ATOM | 1560 | O | ALA | A | 219 | 13.221 | −6.571 | 45.284 | 1.00 | 2.69 | [symbol] |
| ATOM | 1561 | N | GLU | A | 220 | 13.021 | −8.204 | 46.800 | 1.00 | 2.80 | [symbol] |
| ATOM | 1563 | CA | GLU | A | 220 | 12.644 | −7.286 | 47.867 | 1.00 | 3.50 | [symbol] |
| ATOM | 1565 | CB | GLU | A | 220 | 12.332 | −8.043 | 49.165 | 1.00 | 4.18 | [symbol] |
| ATOM | 1568 | CG | GLU | A | 220 | 12.052 | −7.104 | 50.330 | 1.00 | 5.43 | [symbol] |
| ATOM | 1571 | CD | GLU | A | 220 | 11.359 | −7.774 | 51.506 | 1.00 | 7.31 | [symbol] |
| ATOM | 1572 | OE1 | GLU | A | 220 | 11.761 | −8.899 | 51.879 | 1.00 | 9.83 | [symbol] |
| ATOM | 1573 | OE2 | GLU | A | 220 | 10.420 | −7.163 | 52.069 | 1.00 | 6.12 | [symbol] |
| ATOM | 1574 | C | GLU | A | 220 | 13.792 | −6.321 | 48.138 | 1.00 | 3.30 | [symbol] |
| ATOM | 1575 | O | GLU | A | 220 | 14.942 | −6.747 | 48.291 | 1.00 | 3.39 | [symbol] |
| ATOM | 1576 | N | SER | A | 221 | 13.485 | −5.032 | 48.215 | 1.00 | 3.07 | [symbol] |
| ATOM | 1578 | CA | SER | A | 221 | 14.500 | −4.024 | 48.513 | 1.00 | 3.25 | [symbol] |
| ATOM | 1580 | CB | SER | A | 221 | 13.896 | −2.637 | 48.422 | 1.00 | 3.15 | [symbol] |
| ATOM | 1583 | OG | SER | A | 221 | 14.888 | −1.657 | 48.617 | 1.00 | 3.60 | [symbol] |
| ATOM | 1585 | C | SER | A | 221 | 15.103 | −4.174 | 49.913 | 1.00 | 3.41 | [symbol] |
| ATOM | 1586 | O | SER | A | 221 | 14.425 | −4.601 | 50.853 | 1.00 | 2.86 | [symbol] |
| ATOM | 1587 | N | LEU | A | 222 | 16.375 | −3.790 | 50.034 | 1.00 | 3.90 | [symbol] |
| ATOM | 1589 | CA | LEU | A | 222 | 17.070 | −3.766 | 51.321 | 1.00 | 4.56 | [symbol] |
| ATOM | 1591 | CB | LEU | A | 222 | 18.588 | −3.834 | 51.130 | 1.00 | 5.30 | [symbol] |
| ATOM | 1594 | CG | LEU | A | 222 | 19.097 | −4.976 | 50.249 | 1.00 | 7.15 | [symbol] |
| ATOM | 1596 | CD1 | LEU | A | 222 | 20.607 | −4.964 | 50.183 | 1.00 | 9.23 | [symbol] |
| ATOM | 1600 | CD2 | LEU | A | 222 | 18.609 | −6.315 | 50.772 | 1.00 | 8.38 | [symbol] |
| ATOM | 1604 | C | LEU | A | 222 | 16.723 | −2.480 | 52.059 | 1.00 | 4.29 | [symbol] |
| ATOM | 1605 | O | LEU | A | 222 | 17.001 | −2.343 | 53.254 | 1.00 | 4.06 | [symbol] |
| ATOM | 1606 | N | ILE | A | 223 | 16.138 | −1.527 | 51.343 | 1.00 | 3.99 | [symbol] |
| ATOM | 1608 | CA | ILE | A | 223 | 15.724 | −0.267 | 51.944 | 1.00 | 4.02 | [symbol] |
| ATOM | 1610 | CB | ILE | A | 223 | 15.547 | 0.816 | 50.886 | 1.00 | 3.79 | [symbol] |
| ATOM | 1612 | CG1 | ILE | A | 223 | 16.865 | 1.041 | 50.150 | 1.00 | 5.03 | [symbol] |
| ATOM | 1615 | CD1 | ILE | A | 223 | 16.706 | 1.847 | 48.893 | 1.00 | 4.83 | [symbol] |
| ATOM | 1619 | CG2 | ILE | A | 223 | 15.073 | 2.118 | 51.527 | 1.00 | 3.14 | [symbol] |
| ATOM | 1623 | C | ILE | A | 223 | 14.411 | −0.511 | 52.669 | 1.00 | 3.87 | [symbol] |
| ATOM | 1624 | O | ILE | A | 223 | 13.503 | −1.110 | 52.119 | 1.00 | 2.80 | [symbol] |
| ATOM | 1625 | N | SER | A | 224 | 14.343 | −0.082 | 53.923 | 1.00 | 3.63 | [symbol] |
| ATOM | 1627 | CA | SER | A | 224 | 13.162 | −0.286 | 54.758 | 1.00 | 3.85 | [symbol] |
| ATOM | 1629 | CB | SER | A | 224 | 13.353 | 0.467 | 56.075 | 1.00 | 4.59 | [symbol] |
| ATOM | 1632 | OG | SER | A | 224 | 12.305 | 0.153 | 56.970 | 1.00 | 6.66 | [symbol] |
| ATOM | 1634 | C | SER | A | 224 | 11.834 | 0.150 | 54.131 | 1.00 | 3.32 | [symbol] |
| ATOM | 1635 | O | SER | A | 224 | 11.658 | 1.317 | 53.811 | 1.00 | 2.78 | [symbol] |
| ATOM | 1636 | N | SER | A | 225 | 10.913 | −0.801 | 53.972 | 1.00 | 3.00 | [symbol] |
| ATOM | 1638 | CA | SER | A | 225 | 9.545 | −0.514 | 53.494 | 1.00 | 3.09 | [symbol] |
| ATOM | 1640 | CB | SER | A | 225 | 8.874 | 0.469 | 54.462 | 1.00 | 2.88 | [symbol] |
| ATOM | 1643 | OG | SER | A | 225 | 8.729 | −0.106 | 55.745 | 1.00 | 3.17 | [symbol] |
| ATOM | 1645 | C | SER | A | 225 | 9.470 | 0.031 | 52.068 | 1.00 | 3.22 | [symbol] |
| ATOM | 1646 | O | SER | A | 225 | 8.407 | 0.502 | 51.627 | 1.00 | 3.34 | [symbol] |
| ATOM | 1647 | N | ALA | A | 226 | 10.572 | −0.066 | 51.331 | 1.00 | 3.38 | [symbol] |
| ATOM | 1649 | CA | ALA | A | 226 | 10.683 | 0.493 | 49.987 | 1.00 | 3.48 | [symbol] |
| ATOM | 1651 | CB | ALA | A | 226 | 12.139 | 0.766 | 49.672 | 1.00 | 3.48 | [symbol] |
| ATOM | 1655 | C | ALA | A | 226 | 10.060 | −0.368 | 48.879 | 1.00 | 3.36 | [symbol] |
| ATOM | 1656 | O | ALA | A | 226 | 10.057 | 0.036 | 47.717 | 1.00 | 3.94 | [symbol] |
| ATOM | 1657 | N | GLY | A | 227 | 9.531 | −1.534 | 49.244 | 1.00 | 3.22 | [symbol] |
| ATOM | 1659 | CA | GLY | A | 227 | 8.911 | −2.445 | 48.299 | 1.00 | 2.87 | [symbol] |
| ATOM | 1662 | C | GLY | A | 227 | 9.944 | −3.293 | 47.583 | 1.00 | 2.64 | [symbol] |
| ATOM | 1663 | O | GLY | A | 227 | 10.914 | −3.763 | 48.189 | 1.00 | 2.43 | [symbol] |
| ATOM | 1664 | N | GLU | A | 228 | 9.747 | −3.486 | 46.281 | 1.00 | 2.32 | [symbol] |
| ATOM | 1666 | CA | GLU | A | 228 | 10.693 | −4.244 | 45.484 | 1.00 | 2.34 | [symbol] |
| ATOM | 1668 | CB | GLU | A | 228 | 10.039 | −4.814 | 44.223 | 1.00 | 2.52 | [symbol] |
| ATOM | 1671 | CG | GLU | A | 228 | 8.887 | −5.768 | 44.488 | 1.00 | 3.17 | [symbol] |
| ATOM | 1674 | CD | GLU | A | 228 | 8.461 | −6.522 | 43.236 | 1.00 | 4.92 | [symbol] |
| ATOM | 1675 | OE1 | GLU | A | 228 | 8.462 | −5.922 | 42.145 | 1.00 | 5.91 | [symbol] |
| ATOM | 1676 | OE2 | GLU | A | 228 | 8.141 | −7.722 | 43.344 | 1.00 | 6.16 | [symbol] |
| ATOM | 1677 | C | GLU | A | 228 | 11.825 | −3.310 | 45.083 | 1.00 | 2.31 | [symbol] |
| ATOM | 1678 | O | GLU | A | 228 | 11.652 | −2.099 | 45.062 | 1.00 | 2.49 | [symbol] |
| ATOM | 1679 | N | GLY | A | 229 | 12.980 | −3.887 | 44.771 | 1.00 | 2.30 | [symbol] |
| ATOM | 1681 | CA | GLY | A | 229 | 14.119 | −3.135 | 44.286 | 1.00 | 2.20 | [symbol] |
| ATOM | 1684 | C | GLY | A | 229 | 14.778 | −3.869 | 43.125 | 1.00 | 2.15 | [symbol] |
| ATOM | 1685 | O | GLY | A | 229 | 14.360 | −4.973 | 42.754 | 1.00 | 2.15 | [symbol] |
| ATOM | 1686 | N | LEU | A | 230 | 15.812 | −3.254 | 42.561 | 1.00 | 2.22 | [symbol] |
| ATOM | 1688 | CA | LEU | A | 230 | 16.549 | −3.802 | 41.435 | 1.00 | 2.13 | [symbol] |
| ATOM | 1690 | CB | LEU | A | 230 | 16.623 | −2.757 | 40.328 | 1.00 | 2.21 | [symbol] |
| ATOM | 1693 | CG | LEU | A | 230 | 17.162 | −3.248 | 38.983 | 1.00 | 2.18 | [symbol] |
| ATOM | 1695 | CD1 | LEU | A | 230 | 16.210 | −4.268 | 38.363 | 1.00 | 2.50 | [symbol] |
| ATOM | 1699 | CD2 | LEU | A | 230 | 17.388 | −2.068 | 38.043 | 1.00 | 2.60 | [symbol] |
| ATOM | 1703 | C | LEU | A | 230 | 17.956 | −4.182 | 41.901 | 1.00 | 2.15 | [symbol] |
| ATOM | 1704 | O | LEU | A | 230 | 18.621 | −3.385 | 42.549 | 1.00 | 2.20 | [symbol] |
| ATOM | 1705 | N | PHE | A | 231 | 18.396 | −5.394 | 41.580 | 1.00 | 2.05 | [symbol] |
| ATOM | 1707 | CA | PHE | A | 231 | 19.697 | −5.875 | 42.041 | 1.00 | 2.38 | [symbol] |
| ATOM | 1709 | CB | PHE | A | 231 | 19.507 | −7.041 | 43.007 | 1.00 | 2.23 | [symbol] |
| ATOM | 1712 | CG | PHE | A | 231 | 18.813 | −6.666 | 44.269 | 1.00 | 2.69 | [symbol] |
| ATOM | 1713 | CD1 | PHE | A | 231 | 17.443 | −6.483 | 44.302 | 1.00 | 3.21 | [symbol] |
| ATOM | 1715 | CE1 | PHE | A | 231 | 16.807 | −6.139 | 45.480 | 1.00 | 3.94 | [symbol] |
| ATOM | 1717 | CZ | PHE | A | 231 | 17.542 | −5.970 | 46.633 | 1.00 | 2.72 | [symbol] |
| ATOM | 1719 | CE2 | PHE | A | 231 | 18.900 | −6.141 | 46.607 | 1.00 | 3.21 | [symbol] |
| ATOM | 1721 | CD2 | PHE | A | 231 | 19.534 | −6.486 | 45.434 | 1.00 | 2.54 | [symbol] |
| ATOM | 1723 | C | PHE | A | 231 | 20.609 | −6.336 | 40.921 | 1.00 | 2.43 | [symbol] |
| ATOM | 1724 | O | PHE | A | 231 | 20.168 | −6.832 | 39.910 | 1.00 | 2.42 | [symbol] |
| ATOM | 1725 | N | SER | A | 232 | 21.909 | −6.195 | 41.130 | 1.00 | 2.69 | [symbol] |
| ATOM | 1727 | CA | SER | A | 232 | 22.862 | −6.677 | 40.158 | 1.00 | 3.10 | [symbol] |
| ATOM | 1729 | CB | SER | A | 232 | 24.242 | −6.068 | 40.413 | 1.00 | 2.95 | [symbol] |
| ATOM | 1732 | OG | ASER | A | 232 | 24.570 | −6.073 | 41.799 | 0.50 | 5.29 | [symbol] |
| ATOM | 1733 | OG | BSER | A | 232 | 25.105 | −6.433 | 39.353 | 0.50 | 4.49 | [symbol] |
| ATOM | 1736 | C | SER | A | 232 | 22.989 | −8.182 | 40.259 | 1.00 | 2.97 | [symbol] |
| ATOM | 1737 | O | SER | A | 232 | 23.054 | −8.736 | 41.364 | 1.00 | 2.99 | [symbol] |
| ATOM | 1738 | N | LYS | A | 233 | 23.064 | −8.838 | 39.106 | 1.00 | 3.09 | [symbol] |
| ATOM | 1740 | CA | LYS | A | 233 | 23.294 | −10.273 | 39.050 | 1.00 | 3.64 | [symbol] |
| ATOM | 1742 | CB | LYS | A | 233 | 22.732 | −10.849 | 37.748 | 1.00 | 3.60 | [symbol] |
| ATOM | 1745 | CG | LYS | A | 233 | 21.288 | −11.149 | 37.782 | 1.00 | 4.20 | [symbol] |
| ATOM | 1748 | CD | LYS | A | 233 | 20.904 | −12.027 | 36.601 | 1.00 | 4.67 | [symbol] |
| ATOM | 1751 | CE | LYS | A | 233 | 19.415 | −12.173 | 36.504 | 1.00 | 4.71 | [symbol] |
| ATOM | 1754 | NZ | LYS | A | 233 | 19.004 | −13.209 | 35.517 | 1.00 | 4.85 | [symbol] |
| ATOM | 1758 | C | LYS | A | 233 | 24.779 | −10.585 | 39.057 | 1.00 | 3.83 | [symbol] |
| ATOM | 1759 | O | LYS | A | 233 | 25.189 | −11.681 | 39.419 | 1.00 | 4.11 | [symbol] |
| ATOM | 1760 | N | VAL | A | 234 | 25.584 | −9.607 | 38.657 | 1.00 | 4.42 | [symbol] |
| ATOM | 1762 | CA | VAL | A | 234 | 26.996 | −9.819 | 38.406 | 1.00 | 4.40 | [symbol] |
| ATOM | 1764 | CB | VAL | A | 234 | 27.223 | −10.020 | 36.886 | 1.00 | 4.65 | [symbol] |
| ATOM | 1766 | CG1 | VAL | A | 234 | 26.545 | −11.304 | 36.402 | 1.00 | 4.93 | [symbol] |
| ATOM | 1770 | CG2 | VAL | A | 234 | 26.683 | −8.840 | 36.103 | 1.00 | 4.52 | [symbol] |
| ATOM | 1774 | C | VAL | A | 234 | 27.836 | −8.636 | 38.870 | 1.00 | 4.50 | [symbol] |
| ATOM | 1775 | O | VAL | A | 234 | 27.306 | −7.553 | 39.115 | 1.00 | 4.51 | [symbol] |
| ATOM | 1776 | N | ALA | A | 235 | 29.141 | −8.860 | 39.022 | 1.00 | 4.24 | [symbol] |
| ATOM | 1778 | CA | ALA | A | 235 | 30.074 | −7.799 | 39.364 | 1.00 | 3.86 | [symbol] |
| ATOM | 1780 | CB | ALA | A | 235 | 31.387 | −8.386 | 39.903 | 1.00 | 4.11 | [symbol] |
| ATOM | 1784 | C | ALA | A | 235 | 30.358 | −6.973 | 38.120 | 1.00 | 3.50 | [symbol] |
| ATOM | 1785 | O | ALA | A | 235 | 30.673 | −7.530 | 37.077 | 1.00 | 3.56 | [symbol] |
| ATOM | 1786 | N | VAL | A | 236 | 30.255 | −5.653 | 38.235 | 1.00 | 3.07 | [symbol] |
| ATOM | 1788 | CA | VAL | A | 236 | 30.572 | −4.751 | 37.131 | 1.00 | 2.74 | [symbol] |
| ATOM | 1790 | CB | VAL | A | 236 | 29.286 | −4.217 | 36.449 | 1.00 | 2.68 | [symbol] |
| ATOM | 1792 | CG1 | VAL | A | 236 | 28.513 | −5.360 | 35.863 | 1.00 | 3.61 | [symbol] |
| ATOM | 1796 | CG2 | VAL | A | 236 | 28.426 | −3.439 | 37.437 | 1.00 | 2.43 | [symbol] |
| ATOM | 1800 | C | VAL | A | 236 | 31.426 | −3.575 | 37.609 | 1.00 | 2.65 | [symbol] |
| ATOM | 1801 | O | VAL | A | 236 | 31.505 | −3.284 | 38.807 | 1.00 | 2.46 | [symbol] |
| ATOM | 1802 | N | GLY | A | 237 | 32.080 | −2.916 | 36.668 | 1.00 | 2.33 | [symbol] |
| ATOM | 1804 | CA | GLY | A | 237 | 32.831 | −1.722 | 36.973 | 1.00 | 2.18 | [symbol] |
| ATOM | 1807 | C | GLY | A | 237 | 31.965 | −0.487 | 36.807 | 1.00 | 2.29 | [symbol] |
| ATOM | 1808 | O | GLY | A | 237 | 30.770 | −0.572 | 36.490 | 1.00 | 2.08 | [symbol] |
| ATOM | 1809 | N | PRO | A | 238 | 32.567 | 0.675 | 37.015 | 1.00 | 2.31 | [symbol] |
| ATOM | 1810 | CA | PRO | A | 238 | 31.876 | 1.948 | 36.840 | 1.00 | 2.21 | [symbol] |
| ATOM | 1812 | CB | PRO | A | 238 | 32.944 | 2.981 | 37.223 | 1.00 | 2.41 | [symbol] |
| ATOM | 1815 | CG | PRO | A | 238 | 34.019 | 2.248 | 37.840 | 1.00 | 2.81 | [symbol] |
| ATOM | 1818 | CD | PRO | A | 238 | 33.965 | 0.853 | 37.409 | 1.00 | 2.49 | [symbol] |
| ATOM | 1821 | C | PRO | A | 238 | 31.507 | 2.156 | 35.382 | 1.00 | 2.28 | [symbol] |
| ATOM | 1822 | O | PRO | A | 238 | 32.095 | 1.500 | 34.535 | 1.00 | 2.08 | [symbol] |
| ATOM | 1823 | N | ASN | A | 239 | 30.549 | 3.042 | 35.111 | 1.00 | 2.48 | [symbol] |
| ATOM | 1825 | CA | ASN | A | 239 | 30.204 | 3.416 | 33.749 | 1.00 | 2.91 | [symbol] |
| ATOM | 1827 | CB | ASN | A | 239 | 31.457 | 3.843 | 32.981 | 1.00 | 3.61 | [symbol] |
| ATOM | 1830 | CG | ASN | A | 239 | 31.214 | 5.010 | 32.013 | 1.00 | 7.42 | [symbol] |
| ATOM | 1831 | OD1 | ASN | A | 239 | 30.074 | 5.447 | 31.779 | 1.00 | 10.92 | [symbol] |
| ATOM | 1832 | ND2 | ASN | A | 239 | 32.302 | 5.510 | 31.430 | 1.00 | 11.07 | [symbol] |
| ATOM | 1835 | C | ASN | A | 239 | 29.508 | 2.279 | 32.992 | 1.00 | 2.43 | [symbol] |
| ATOM | 1836 | O | ASN | A | 239 | 29.484 | 2.301 | 31.763 | 1.00 | 2.65 | [symbol] |
| ATOM | 1837 | N | THR | A | 240 | 28.933 | 1.315 | 33.708 | 1.00 | 2.07 | [symbol] |
| ATOM | 1839 | CA | THR | A | 240 | 28.302 | 0.157 | 33.059 | 1.00 | 2.01 | [symbol] |
| ATOM | 1841 | CB | THR | A | 240 | 28.632 | −1.143 | 33.799 | 1.00 | 2.12 | [symbol] |
| ATOM | 1843 | OG1 | THR | A | 240 | 30.051 | −1.268 | 33.983 | 1.00 | 2.28 | [symbol] |
| ATOM | 1845 | CG2 | THR | A | 240 | 28.262 | −2.351 | 32.948 | 1.00 | 2.11 | [symbol] |
| ATOM | 1849 | C | THR | A | 240 | 26.777 | 0.275 | 32.950 | 1.00 | 2.04 | [symbol] |
| ATOM | 1850 | O | THR | A | 240 | 26.073 | 0.443 | 33.935 | 1.00 | 2.06 | [symbol] |
| ATOM | 1851 | N | VAL | A | 241 | 26.262 | 0.166 | 31.738 | 1.00 | 2.00 | [symbol] |
| ATOM | 1853 | CA | VAL | A | 241 | 24.815 | 0.177 | 31.570 | 1.00 | 2.01 | [symbol] |
| ATOM | 1855 | CB | VAL | A | 241 | 24.419 | 0.335 | 30.105 | 1.00 | 2.05 | [symbol] |
| ATOM | 1857 | CG1 | VAL | A | 241 | 22.902 | 0.169 | 29.941 | 1.00 | 2.07 | [symbol] |
| ATOM | 1861 | CG2 | VAL | A | 241 | 24.863 | 1.684 | 29.579 | 1.00 | 2.18 | [symbol] |
| ATOM | 1865 | C | VAL | A | 241 | 24.262 | −1.130 | 32.134 | 1.00 | 2.16 | [symbol] |
| ATOM | 1866 | O | VAL | A | 241 | 24.753 | −2.217 | 31.809 | 1.00 | 2.29 | [symbol] |
| ATOM | 1867 | N | MET | A | 242 | 23.224 | −1.036 | 32.958 | 1.00 | 2.59 | [symbol] |
| ATOM | 1869 | CA | MET | A | 242 | 22.672 | −2.226 | 33.615 | 1.00 | 2.76 | [symbol] |
| ATOM | 1871 | CB | MET | A | 242 | 22.756 | −2.078 | 35.126 | 1.00 | 2.81 | [symbol] |
| ATOM | 1874 | CG | MET | A | 242 | 24.163 | −1.918 | 35.634 | 1.00 | 3.09 | [symbol] |
| ATOM | 1877 | SD | MET | A | 242 | 25.119 | −3.426 | 35.424 | 1.00 | 3.66 | [symbol] |
| ATOM | 1878 | CE | MET | A | 242 | 24.649 | −4.347 | 36.888 | 1.00 | 3.91 | [symbol] |
| ATOM | 1882 | C | MET | A | 242 | 21.224 | −2.534 | 33.297 | 1.00 | 2.34 | [symbol] |
| ATOM | 1883 | O | MET | A | 242 | 20.839 | −3.689 | 33.299 | 1.00 | 2.18 | [symbol] |
| ATOM | 1884 | N | SER | A | 243 | 20.428 | −1.493 | 33.089 | 1.00 | 2.43 | [symbol] |
| ATOM | 1886 | CA | SER | A | 243 | 18.988 | −1.644 | 32.937 | 1.00 | 2.80 | [symbol] |
| ATOM | 1888 | CB | SER | A | 243 | 18.353 | −1.584 | 34.332 | 1.00 | 2.79 | [symbol] |
| ATOM | 1891 | OG | SER | A | 243 | 16.985 | −1.910 | 34.357 | 1.00 | 3.90 | [symbol] |
| ATOM | 1893 | C | SER | A | 243 | 18.430 | −0.532 | 32.059 | 1.00 | 2.74 | [symbol] |
| ATOM | 1894 | O | SER | A | 243 | 19.043 | 0.516 | 31.890 | 1.00 | 3.19 | [symbol] |
| ATOM | 1895 | N | PHE | A | 244 | 17.249 | −0.764 | 31.516 | 1.00 | 2.66 | [symbol] |
| ATOM | 1897 | CA | PHE | A | 244 | 16.602 | 0.200 | 30.632 | 1.00 | 2.43 | [symbol] |
| ATOM | 1899 | CB | PHE | A | 244 | 16.358 | −0.446 | 29.280 | 1.00 | 2.19 | [symbol] |
| ATOM | 1902 | CG | PHE | A | 244 | 17.611 | −0.977 | 28.664 | 1.00 | 2.28 | [symbol] |
| ATOM | 1903 | CD1 | PHE | A | 244 | 17.793 | −2.334 | 28.481 | 1.00 | 2.21 | [symbol] |
| ATOM | 1905 | CE1 | PHE | A | 244 | 18.984 | −2.818 | 27.945 | 1.00 | 2.25 | [symbol] |
| ATOM | 1907 | CZ | PHE | A | 244 | 19.985 | −1.948 | 27.588 | 1.00 | 2.16 | [symbol] |
| ATOM | 1909 | CE2 | PHE | A | 244 | 19.814 | −0.595 | 27.763 | 1.00 | 2.45 | [symbol] |
| ATOM | 1911 | CD2 | PHE | A | 244 | 18.628 | −0.111 | 28.298 | 1.00 | 2.35 | [symbol] |
| ATOM | 1913 | C | PHE | A | 244 | 15.324 | 0.716 | 31.253 | 1.00 | 2.17 | [symbol] |
| ATOM | 1914 | O | PHE | A | 244 | 14.640 | −0.013 | 31.958 | 1.00 | 2.24 | [symbol] |
| ATOM | 1915 | N | TYR | A | 245 | 15.035 | 1.991 | 31.009 | 1.00 | 2.18 | [symbol] |
| ATOM | 1917 | CA | TYR | A | 245 | 13.862 | 2.651 | 31.560 | 1.00 | 2.15 | [symbol] |
| ATOM | 1919 | CB | TYR | A | 245 | 14.256 | 3.924 | 32.353 | 1.00 | 2.20 | [symbol] |
| ATOM | 1922 | CG | TYR | A | 245 | 13.301 | 4.321 | 33.471 | 1.00 | 2.00 | [symbol] |
| ATOM | 1923 | CD1 | TYR | A | 245 | 13.712 | 4.344 | 34.795 | 1.00 | 2.00 | [symbol] |
| ATOM | 1925 | CE1 | TYR | A | 245 | 12.848 | 4.708 | 35.812 | 1.00 | 2.07 | [symbol] |
| ATOM | 1927 | CZ | TYR | A | 245 | 11.548 | 5.070 | 35.516 | 1.00 | 2.37 | [symbol] |
| ATOM | 1928 | OH | TYR | A | 245 | 10.675 | 5.430 | 36.522 | 1.00 | 2.32 | [symbol] |
| ATOM | 1930 | CE2 | TYR | A | 245 | 11.121 | 5.048 | 34.214 | 1.00 | 2.16 | [symbol] |
| ATOM | 1932 | CD2 | TYR | A | 245 | 11.988 | 4.684 | 33.201 | 1.00 | 2.44 | [symbol] |
| ATOM | 1934 | C | TYR | A | 245 | 12.932 | 2.994 | 30.402 | 1.00 | 2.45 | [symbol] |
| ATOM | 1935 | O | TYR | A | 245 | 12.892 | 4.136 | 29.945 | 1.00 | 2.43 | [symbol] |
| ATOM | 1936 | N | ASN | A | 246 | 12.238 | 1.977 | 29.890 | 1.00 | 2.77 | [symbol] |
| ATOM | 1938 | CA | ASN | A | 246 | 11.219 | 2.175 | 28.862 | 1.00 | 3.04 | [symbol] |
| ATOM | 1940 | CB | ASN | A | 246 | 11.057 | 0.938 | 27.976 | 1.00 | 2.93 | [symbol] |
| ATOM | 1943 | CG | ASN | A | 246 | 10.037 | 1.154 | 26.861 | 1.00 | 2.79 | [symbol] |
| ATOM | 1944 | OD1 | ASN | A | 246 | 10.093 | 2.157 | 26.148 | 1.00 | 2.29 | [symbol] |
| ATOM | 1945 | ND2 | ASN | A | 246 | 9.086 | 0.220 | 26.721 | 1.00 | 2.93 | [symbol] |
| ATOM | 1948 | C | ASN | A | 246 | 9.895 | 2.460 | 29.556 | 1.00 | 3.31 | [symbol] |
| ATOM | 1949 | O | ASN | A | 246 | 9.744 | 2.209 | 30.747 | 1.00 | 3.44 | [symbol] |
| ATOM | 1950 | N | GLY | A | 247 | 8.944 | 3.003 | 28.814 | 1.00 | 3.66 | [symbol] |
| ATOM | 1952 | CA | GLY | A | 247 | 7.616 | 3.268 | 29.336 | 1.00 | 3.80 | [symbol] |
| ATOM | 1955 | C | GLY | A | 247 | 6.799 | 4.017 | 28.309 | 1.00 | 4.27 | [symbol] |
| ATOM | 1956 | O | GLY | A | 247 | 7.253 | 4.225 | 27.178 | 1.00 | 4.28 | [symbol] |
| ATOM | 1957 | N | VAL | A | 248 | 5.584 | 4.396 | 28.697 | 1.00 | 4.32 | [symbol] |
| ATOM | 1959 | CA | VAL | A | 248 | 4.708 | 5.179 | 27.852 | 1.00 | 4.50 | [symbol] |
| ATOM | 1961 | CB | VAL | A | 248 | 3.233 | 5.103 | 28.304 | 1.00 | 4.48 | [symbol] |
| ATOM | 1963 | CG1 | VAL | A | 248 | 2.745 | 3.670 | 28.288 | 1.00 | 5.63 | [symbol] |
| ATOM | 1967 | CG2 | VAL | A | 248 | 3.023 | 5.702 | 29.664 | 1.00 | 4.96 | [symbol] |
| ATOM | 1971 | C | VAL | A | 248 | 5.195 | 6.620 | 27.881 | 1.00 | 4.37 | [symbol] |
| ATOM | 1972 | O | VAL | A | 248 | 5.774 | 7.068 | 28.864 | 1.00 | 4.09 | [symbol] |
| ATOM | 1973 | N | ARG | A | 249 | 4.974 | 7.348 | 26.795 | 1.00 | 4.02 | [symbol] |
| ATOM | 1975 | CA | ARG | A | 249 | 5.419 | 8.733 | 26.726 | 1.00 | 4.38 | [symbol] |
| ATOM | 1977 | CB | ARG | A | 249 | 6.013 | 9.022 | 25.353 | 1.00 | 5.06 | [symbol] |
| ATOM | 1980 | CG | ARG | A | 249 | 7.450 | 8.604 | 25.238 | 1.00 | 6.52 | [symbol] |
| ATOM | 1983 | CD | ARG | A | 249 | 7.815 | 7.997 | 23.918 | 1.00 | 9.42 | [symbol] |
| ATOM | 1986 | NE | ARG | A | 249 | 8.843 | 6.985 | 24.094 | 1.00 | 11.70 | [symbol] |
| ATOM | 1988 | CZ | ARG | A | 249 | 10.093 | 7.116 | 23.696 | 1.00 | 13.30 | [symbol] |
| ATOM | 1989 | NH1 | ARG | A | 249 | 10.475 | 8.216 | 23.070 | 1.00 | 15.18 | [symbol] |
| ATOM | 1992 | NH2 | ARG | A | 249 | 10.965 | 6.143 | 23.922 | 1.00 | 13.35 | [symbol] |
| ATOM | 1995 | C | ARG | A | 249 | 4.248 | 9.675 | 26.983 | 1.00 | 4.18 | [symbol] |
| ATOM | 1996 | O | ARG | A | 249 | 3.199 | 9.560 | 26.346 | 1.00 | 3.34 | [symbol] |
| ATOM | 1997 | N | ILE | A | 250 | 4.429 | 10.615 | 27.902 | 1.00 | 3.39 | [symbol] |
| ATOM | 1999 | CA | ILE | A | 250 | 3.389 | 11.580 | 28.226 | 1.00 | 3.73 | [symbol] |
| ATOM | 2001 | CB | ILE | A | 250 | 2.611 | 11.120 | 29.460 | 1.00 | 4.31 | [symbol] |
| ATOM | 2003 | CG1 | ILE | A | 250 | 3.553 | 10.933 | 30.639 | 1.00 | 4.63 | [symbol] |
| ATOM | 2006 | CD1 | ILE | A | 250 | 2.844 | 10.570 | 31.927 | 1.00 | 5.32 | [symbol] |
| ATOM | 2010 | CG2 | ILE | A | 250 | 1.887 | 9.813 | 29.164 | 1.00 | 5.16 | [symbol] |
| ATOM | 2014 | C | ILE | A | 250 | 3.992 | 12.972 | 28.438 | 1.00 | 3.77 | [symbol] |
| ATOM | 2015 | O | ILE | A | 250 | 5.213 | 13.161 | 28.334 | 1.00 | 3.89 | [symbol] |
| ATOM | 2016 | N | THR | A | 251 | 3.148 | 13.951 | 28.740 | 1.00 | 3.68 | [symbol] |
| ATOM | 2018 | CA | THR | A | 251 | 3.637 | 15.312 | 28.956 | 1.00 | 3.84 | [symbol] |
| ATOM | 2020 | CB | THR | A | 251 | 2.637 | 16.325 | 28.398 | 1.00 | 3.91 | [symbol] |
| ATOM | 2022 | OG1 | THR | A | 251 | 1.476 | 16.366 | 29.245 | 1.00 | 2.98 | [symbol] |
| ATOM | 2024 | CG2 | THR | A | 251 | 2.135 | 15.916 | 27.032 | 1.00 | 3.69 | [symbol] |
| ATOM | 2028 | C | THR | A | 251 | 3.874 | 15.648 | 30.430 | 1.00 | 4.23 | [symbol] |
| ATOM | 2029 | O | THR | A | 251 | 3.304 | 15.034 | 31.329 | 1.00 | 3.85 | [symbol] |
| ATOM | 2030 | N | HIS | A | 252 | 4.724 | 16.644 | 30.656 | 1.00 | 4.92 | [symbol] |
| ATOM | 2032 | CA | HIS | A | 252 | 4.997 | 17.135 | 31.987 | 1.00 | 5.37 | [symbol] |
| ATOM | 2034 | CB | HIS | A | 252 | 6.129 | 18.167 | 31.939 | 1.00 | 5.54 | [symbol] |
| ATOM | 2037 | CG | HIS | A | 252 | 7.471 | 17.579 | 31.608 | 1.00 | 5.74 | [symbol] |
| ATOM | 2038 | ND1 | HIS | A | 252 | 8.365 | 17.169 | 32.572 | 1.00 | 6.59 | [symbol] |
| ATOM | 2040 | CE1 | HIS | A | 252 | 9.454 | 16.699 | 31.990 | 1.00 | 5.78 | [symbol] |
| ATOM | 2042 | NE2 | HIS | A | 252 | 9.301 | 16.796 | 30.683 | 1.00 | 6.87 | [symbol] |
| ATOM | 2044 | CD2 | HIS | A | 252 | 8.066 | 17.336 | 30.417 | 1.00 | 6.09 | [symbol] |
| ATOM | 2046 | C | HIS | A | 252 | 3.730 | 17.755 | 32.590 | 1.00 | 5.91 | [symbol] |
| ATOM | 2047 | O | HIS | A | 252 | 3.529 | 17.740 | 33.783 | 1.00 | 6.19 | [symbol] |
| ATOM | 2048 | N | GLN | A | 253 | 2.858 | 18.287 | 31.748 | 1.00 | 6.66 | [symbol] |
| ATOM | 2050 | CA | GLN | A | 253 | 1.646 | 18.938 | 32.236 | 1.00 | 7.22 | [symbol] |
| ATOM | 2052 | CB | GLN | A | 253 | 0.923 | 19.618 | 31.094 | 1.00 | 7.93 | [symbol] |
| ATOM | 2055 | CG | GLN | A | 253 | 1.585 | 20.884 | 30.621 | 1.00 | 9.76 | [symbol] |
| ATOM | 2058 | CD | GLN | A | 253 | 2.804 | 20.650 | 29.728 | 1.00 | 11.24 | [symbol] |
| ATOM | 2059 | OE1 | GLN | A | 253 | 3.202 | 21.534 | 28.981 | 1.00 | 12.89 | [symbol] |
| ATOM | 2060 | NE2 | GLN | A | 253 | 3.384 | 19.472 | 29.800 | 1.00 | 13.47 | [symbol] |
| ATOM | 2063 | C | GLN | A | 253 | 0.683 | 17.970 | 32.877 | 1.00 | 6.97 | [symbol] |
| ATOM | 2064 | O | GLN | A | 253 | 0.187 | 18.204 | 33.982 | 1.00 | 7.62 | [symbol] |
| ATOM | 2065 | N | GLU | A | 254 | 0.418 | 16.887 | 32.168 | 1.00 | 6.45 | [symbol] |
| ATOM | 2067 | CA | GLU | A | 254 | −0.470 | 15.849 | 32.654 | 1.00 | 7.11 | [symbol] |
| ATOM | 2069 | CB | GLU | A | 254 | −0.530 | 14.696 | 31.648 | 1.00 | 7.42 | [symbol] |
| ATOM | 2072 | CG | GLU | A | 254 | −1.409 | 13.521 | 32.075 | 1.00 | 9.67 | [symbol] |
| ATOM | 2075 | CD | GLU | A | 254 | −1.514 | 12.426 | 31.008 | 1.00 | 12.85 | [symbol] |
| ATOM | 2076 | OE1 | GLU | A | 254 | −0.897 | 12.561 | 29.925 | 1.00 | 12.24 | [symbol] |
| ATOM | 2077 | OE2 | GLU | A | 254 | −2.230 | 11.423 | 31.249 | 1.00 | 15.26 | [symbol] |
| ATOM | 2078 | C | GLU | A | 254 | 0.045 | 15.333 | 33.988 | 1.00 | 6.48 | [symbol] |
| ATOM | 2079 | O | GLU | A | 254 | −0.725 | 15.114 | 34.913 | 1.00 | 6.44 | [symbol] |
| ATOM | 2080 | N | VAL | A | 255 | 1.364 | 15.155 | 34.075 | 1.00 | 6.59 | [symbol] |
| ATOM | 2082 | CA | VAL | A | 255 | 2.005 | 14.650 | 35.278 | 1.00 | 6.49 | [symbol] |
| ATOM | 2084 | CB | VAL | A | 255 | 3.505 | 14.345 | 35.012 | 1.00 | 6.42 | [symbol] |
| ATOM | 2086 | CG1 | VAL | A | 255 | 4.236 | 14.063 | 36.290 | 1.00 | 6.18 | [symbol] |
| ATOM | 2090 | CG2 | VAL | A | 255 | 3.647 | 13.158 | 34.066 | 1.00 | 5.96 | [symbol] |
| ATOM | 2094 | C | VAL | A | 255 | 1.853 | 15.651 | 36.439 | 1.00 | 6.75 | [symbol] |
| ATOM | 2095 | O | VAL | A | 255 | 1.463 | 15.273 | 37.531 | 1.00 | 6.38 | [symbol] |
| ATOM | 2096 | N | ASP | A | 256 | 2.126 | 16.929 | 36.181 | 1.00 | 7.52 | [symbol] |
| ATOM | 2098 | CA | ASP | A | 256 | 2.012 | 17.982 | 37.199 | 1.00 | 8.01 | [symbol] |
| ATOM | 2100 | CB | ASP | A | 256 | 2.583 | 19.306 | 36.678 | 1.00 | 8.71 | [symbol] |
| ATOM | 2103 | CG | ASP | A | 256 | 4.066 | 19.221 | 36.343 | 1.00 | 10.13 | [symbol] |
| ATOM | 2104 | OD1 | ASP | A | 256 | 4.731 | 18.286 | 36.812 | 1.00 | 11.21 | [symbol] |
| ATOM | 2105 | OD2 | ASP | A | 256 | 4.660 | 20.027 | 35.596 | 1.00 | 13.23 | [symbol] |
| ATOM | 2106 | C | ASP | A | 256 | 0.565 | 18.216 | 37.629 | 1.00 | 7.94 | [symbol] |
| ATOM | 2107 | O | ASP | A | 256 | 0.317 | 18.769 | 38.702 | 1.00 | 8.89 | [symbol] |
| ATOM | 2108 | N | SER | A | 257 | −0.382 | 17.788 | 36.802 | 1.00 | 7.19 | [symbol] |
| ATOM | 2110 | CA | SER | A | 257 | −1.799 | 17.999 | 37.086 | 1.00 | 6.69 | [symbol] |
| ATOM | 2112 | CB | SER | A | 257 | −2.549 | 18.295 | 35.789 | 1.00 | 6.90 | [symbol] |
| ATOM | 2115 | OG | SER | A | 257 | −2.974 | 17.081 | 35.197 | 1.00 | 7.47 | [symbol] |
| ATOM | 2117 | C | SER | A | 257 | −2.443 | 16.794 | 37.772 | 1.00 | 6.14 | [symbol] |
| ATOM | 2118 | O | SER | A | 257 | −3.653 | 16.773 | 37.998 | 1.00 | 6.30 | [symbol] |
| ATOM | 2119 | N | ARG | A | 258 | −1.650 | 15.783 | 38.113 | 1.00 | 5.33 | [symbol] |
| ATOM | 2121 | CA | ARG | A | 258 | −2.224 | 14.601 | 38.751 | 1.00 | 4.73 | [symbol] |
| ATOM | 2123 | CB | ARG | A | 258 | −2.178 | 13.397 | 37.822 | 1.00 | 4.74 | [symbol] |
| ATOM | 2126 | CG | ARG | A | 258 | −0.801 | 12.948 | 37.443 | 1.00 | 4.66 | [symbol] |
| ATOM | 2129 | CD | ARG | A | 258 | −0.806 | 11.964 | 36.291 | 1.00 | 4.23 | [symbol] |
| ATOM | 2132 | NE | ARG | A | 258 | 0.439 | 11.225 | 36.181 | 1.00 | 3.76 | [symbol] |
| ATOM | 2134 | CZ | ARG | A | 258 | 0.678 | 10.280 | 35.288 | 1.00 | 3.91 | [symbol] |
| ATOM | 2135 | NH1 | ARG | A | 258 | −0.253 | 9.948 | 34.393 | 1.00 | 3.83 | [symbol] |
| ATOM | 2138 | NH2 | ARG | A | 258 | 1.857 | 9.672 | 35.282 | 1.00 | 4.26 | [symbol] |
| ATOM | 2141 | C | ARG | A | 258 | −1.551 | 14.266 | 40.077 | 1.00 | 4.32 | [symbol] |
| ATOM | 2142 | O | ARG | A | 258 | −0.455 | 14.729 | 40.356 | 1.00 | 3.68 | [symbol] |
| ATOM | 2143 | N | ASP | A | 259 | −2.254 | 13.461 | 40.868 | 1.00 | 4.17 | [symbol] |
| ATOM | 2145 | CA | ASP | A | 259 | −1.831 | 13.025 | 42.202 | 1.00 | 3.92 | [symbol] |
| ATOM | 2147 | CB | ASP | A | 259 | −2.930 | 12.122 | 42.753 | 1.00 | 4.24 | [symbol] |
| ATOM | 2150 | CG | ASP | A | 259 | −2.800 | 11.847 | 44.221 | 1.00 | 5.85 | [symbol] |
| ATOM | 2151 | OD1 | ASP | A | 259 | −1.852 | 11.160 | 44.626 | 1.00 | 6.94 | [symbol] |
| ATOM | 2152 | OD2 | ASP | A | 259 | −3.658 | 12.248 | 45.031 | 1.00 | 9.13 | [symbol] |
| ATOM | 2153 | C | ASP | A | 259 | −0.514 | 12.255 | 42.188 | 1.00 | 3.64 | [symbol] |
| ATOM | 2154 | O | ASP | A | 259 | −0.214 | 11.522 | 41.245 | 1.00 | 3.11 | [symbol] |
| ATOM | 2155 | N | TRP | A | 260 | 0.269 | 12.394 | 43.259 | 1.00 | 3.86 | [symbol] |
| ATOM | 2157 | CA | TRP | A | 260 | 1.550 | 11.696 | 43.325 | 1.00 | 3.80 | [symbol] |
| ATOM | 2159 | CB | TRP | A | 260 | 2.322 | 12.060 | 44.581 | 1.00 | 4.34 | [symbol] |
| ATOM | 2162 | CG | TRP | A | 260 | 3.204 | 13.248 | 44.416 | 1.00 | 5.06 | [symbol] |
| ATOM | 2163 | CD1 | TRP | A | 260 | 2.993 | 14.485 | 44.936 | 1.00 | 5.61 | [symbol] |
| ATOM | 2165 | NE1 | TRP | A | 260 | 4.011 | 15.332 | 44.573 | 1.00 | 5.32 | [symbol] |
| ATOM | 2167 | CE2 | TRP | A | 260 | 4.916 | 14.639 | 43.812 | 1.00 | 5.59 | [symbol] |
| ATOM | 2168 | CD2 | TRP | A | 260 | 4.443 | 13.320 | 43.695 | 1.00 | 4.92 | [symbol] |
| ATOM | 2169 | CE3 | TRP | A | 260 | 5.196 | 12.406 | 42.945 | 1.00 | 5.71 | [symbol] |
| ATOM | 2171 | CZ3 | TRP | A | 260 | 6.383 | 12.839 | 42.357 | 1.00 | 6.16 | [symbol] |
| ATOM | 2173 | CH2 | TRP | A | 260 | 6.822 | 14.155 | 42.506 | 1.00 | 5.94 | [symbol] |
| ATOM | 2175 | CZ2 | TRP | A | 260 | 6.103 | 15.066 | 43.222 | 1.00 | 6.33 | [symbol] |
| ATOM | 2177 | C | TRP | A | 260 | 1.374 | 10.193 | 43.279 | 1.00 | 3.37 | [symbol] |
| ATOM | 2178 | O | TRP | A | 260 | 2.280 | 9.476 | 42.883 | 1.00 | 4.00 | [symbol] |
| ATOM | 2179 | N | ALA | A | 261 | 0.215 | 9.715 | 43.702 | 1.00 | 2.79 | [symbol] |
| ATOM | 2181 | CA | ALA | A | 261 | −0.050 | 8.292 | 43.691 | 1.00 | 2.81 | [symbol] |
| ATOM | 2183 | CB | ALA | A | 261 | −1.418 | 8.005 | 44.207 | 1.00 | 3.00 | [symbol] |
| ATOM | 2187 | C | ALA | A | 261 | 0.115 | 7.752 | 42.270 | 1.00 | 2.68 | [symbol] |
| ATOM | 2188 | O | ALA | A | 261 | 0.342 | 6.565 | 42.093 | 1.00 | 2.83 | [symbol] |
| ATOM | 2189 | N | LEU | A | 262 | 0.022 | 8.633 | 41.270 | 1.00 | 2.70 | [symbol] |
| ATOM | 2191 | CA | LEU | A | 262 | 0.198 | 8.252 | 39.866 | 1.00 | 2.95 | [symbol] |
| ATOM | 2193 | CB | LEU | A | 262 | −0.841 | 8.985 | 38.993 | 1.00 | 2.95 | [symbol] |
| ATOM | 2196 | CG | LEU | A | 262 | −2.312 | 8.808 | 39.386 | 1.00 | 3.70 | [symbol] |
| ATOM | 2198 | CD1 | LEU | A | 262 | −3.220 | 9.716 | 38.576 | 1.00 | 4.17 | [symbol] |
| ATOM | 2202 | CD2 | LEU | A | 262 | −2.738 | 7.384 | 39.178 | 1.00 | 4.22 | [symbol] |
| ATOM | 2206 | C | LEU | A | 262 | 1.582 | 8.582 | 39.304 | 1.00 | 3.18 | [symbol] |
| ATOM | 2207 | O | LEU | A | 262 | 1.877 | 8.216 | 38.161 | 1.00 | 3.23 | [symbol] |
| ATOM | 2208 | N | ASN | A | 263 | 2.427 | 9.247 | 40.098 | 1.00 | 3.18 | [symbol] |
| ATOM | 2210 | CA | ASN | A | 263 | 3.719 | 9.748 | 39.623 | 1.00 | 3.19 | [symbol] |
| ATOM | 2212 | CB | ASN | A | 263 | 3.757 | 11.274 | 39.771 | 1.00 | 3.19 | [symbol] |
| ATOM | 2215 | CG | ASN | A | 263 | 2.767 | 11.976 | 38.847 | 1.00 | 3.20 | [symbol] |
| ATOM | 2216 | OD1 | ASN | A | 263 | 2.454 | 11.479 | 37.762 | 1.00 | 3.60 | [symbol] |
| ATOM | 2217 | ND2 | ASN | A | 263 | 2.263 | 13.132 | 39.273 | 1.00 | 2.78 | [symbol] |
| ATOM | 2220 | C | ASN | A | 263 | 4.956 | 9.112 | 40.279 | 1.00 | 3.27 | [symbol] |
| ATOM | 2221 | O | ASN | A | 263 | 6.022 | 9.740 | 40.386 | 1.00 | 3.08 | [symbol] |
| ATOM | 2222 | N | GLY | A | 264 | 4.821 | 7.874 | 40.742 | 1.00 | 3.23 | [symbol] |
| ATOM | 2224 | CA | GLY | A | 264 | 5.955 | 7.159 | 41.309 | 1.00 | 2.91 | [symbol] |
| ATOM | 2227 | C | GLY | A | 264 | 7.028 | 6.781 | 40.291 | 1.00 | 2.93 | [symbol] |
| ATOM | 2228 | O | GLY | A | 264 | 8.228 | 6.724 | 40.628 | 1.00 | 3.18 | [symbol] |
| ATOM | 2229 | N | ASN | A | 265 | 6.612 | 6.529 | 39.044 | 1.00 | 2.70 | [symbol] |
| ATOM | 2231 | CA | ASN | A | 265 | 7.515 | 6.059 | 37.998 | 1.00 | 2.83 | [symbol] |
| ATOM | 2233 | CB | ASN | A | 265 | 6.995 | 4.736 | 37.440 | 1.00 | 3.16 | [symbol] |
| ATOM | 2236 | CG | ASN | A | 265 | 7.070 | 3.609 | 38.466 | 1.00 | 4.64 | [symbol] |
| ATOM | 2237 | OD1 | ASN | A | 265 | 7.723 | 3.733 | 39.519 | 1.00 | 6.97 | [symbol] |
| ATOM | 2238 | ND2 | ASN | A | 265 | 6.423 | 2.499 | 38.158 | 1.00 | 5.90 | [symbol] |
| ATOM | 2241 | C | ASN | A | 265 | 7.782 | 7.030 | 36.854 | 1.00 | 2.63 | [symbol] |
| ATOM | 2242 | O | ASN | A | 265 | 8.411 | 6.670 | 35.868 | 1.00 | 3.13 | [symbol] |
| ATOM | 2243 | N | THR | A | 266 | 7.312 | 8.260 | 36.975 | 1.00 | 2.76 | [symbol] |
| ATOM | 2245 | CA | THR | A | 266 | 7.534 | 9.243 | 35.928 | 1.00 | 2.53 | [symbol] |
| ATOM | 2247 | CB | THR | A | 266 | 6.657 | 10.449 | 36.156 | 1.00 | 2.58 | [symbol] |
| ATOM | 2249 | OG1 | THR | A | 266 | 6.755 | 10.851 | 37.536 | 1.00 | 2.28 | [symbol] |
| ATOM | 2251 | CG2 | THR | A | 266 | 5.182 | 10.097 | 35.948 | 1.00 | 2.42 | [symbol] |
| ATOM | 2255 | C | THR | A | 266 | 8.988 | 9.687 | 35.950 | 1.00 | 2.81 | [symbol] |
| ATOM | 2256 | O | THR | A | 266 | 9.516 | 10.009 | 37.015 | 1.00 | 3.23 | [symbol] |
| ATOM | 2257 | N | LEU | A | 267 | 9.606 | 9.711 | 34.775 | 1.00 | 2.94 | [symbol] |
| ATOM | 2259 | CA | LEU | A | 267 | 10.987 | 10.109 | 34.596 | 1.00 | 3.68 | [symbol] |
| ATOM | 2261 | CB | LEU | A | 267 | 11.856 | 8.890 | 34.312 | 1.00 | 3.83 | [symbol] |
| ATOM | 2264 | CG | LEU | A | 267 | 13.291 | 9.198 | 33.862 | 1.00 | 3.83 | [symbol] |
| ATOM | 2266 | CD1 | LEU | A | 267 | 14.115 | 9.914 | 34.943 | 1.00 | 4.78 | [symbol] |
| ATOM | 2270 | CD2 | LEU | A | 267 | 13.946 | 7.910 | 33.437 | 1.00 | 4.52 | [symbol] |
| ATOM | 2274 | C | LEU | A | 267 | 11.056 | 11.061 | 33.421 | 1.00 | 4.61 | [symbol] |
| ATOM | 2275 | O | LEU | A | 267 | 10.514 | 10.764 | 32.334 | 1.00 | 3.96 | [symbol] |
| ATOM | 2276 | N | SER | A | 268 | 11.692 | 12.210 | 33.629 | 1.00 | 5.56 | [symbol] |
| ATOM | 2278 | CA | SER | A | 268 | 11.786 | 13.203 | 32.563 | 1.00 | 6.81 | [symbol] |
| ATOM | 2280 | CB | SER | A | 268 | 12.141 | 14.598 | 33.129 | 1.00 | 6.96 | [symbol] |
| ATOM | 2283 | OG | ASER | A | 268 | 11.251 | 14.979 | 34.133 | 0.50 | 7.90 | [symbol] |
| ATOM | 2284 | OG | BSER | A | 268 | 12.920 | 15.273 | 32.173 | 0.50 | 7.34 | [symbol] |
| ATOM | 2287 | C | SER | A | 268 | 12.801 | 12.694 | 31.527 | 1.00 | 7.87 | [symbol] |
| ATOM | 2288 | O | SER | A | 268 | 13.968 | 12.392 | 31.860 | 1.00 | 8.21 | [symbol] |
| ATOM | 2289 | N | LEU | A | 269 | 12.345 | 12.574 | 30.289 | 1.00 | 9.28 | [symbol] |
| ATOM | 2291 | CA | LEU | A | 269 | 13.168 | 12.087 | 29.190 | 1.00 | 10.67 | [symbol] |
| ATOM | 2293 | CB | LEU | A | 269 | 12.292 | 11.404 | 28.140 | 1.00 | 10.48 | [symbol] |
| ATOM | 2296 | CG | LEU | A | 269 | 12.909 | 10.303 | 27.274 | 1.00 | 11.51 | [symbol] |
| ATOM | 2298 | CD1 | LEU | A | 269 | 12.263 | 10.333 | 25.897 | 1.00 | 11.38 | [symbol] |
| ATOM | 2302 | CD2 | LEU | A | 269 | 14.419 | 10.399 | 27.144 | 1.00 | 12.54 | [symbol] |
| ATOM | 2306 | C | LEU | A | 269 | 13.918 | 13.255 | 28.554 | 1.00 | 11.61 | [symbol] |
| ATOM | 2307 | O | LEU | A | 269 | 15.148 | 13.263 | 28.471 | 1.00 | 11.86 | [symbol] |
| ATOM | 2308 | N | ASP | A | 270 | 13.152 | 14.239 | 28.114 | 1.00 | 12.80 | [symbol] |
| ATOM | 2310 | CA | ASP | A | 270 | 13.676 | 15.440 | 27.491 | 1.00 | 13.69 | [symbol] |
| ATOM | 2312 | CB | ASP | A | 270 | 13.689 | 15.311 | 25.962 | 1.00 | 13.95 | [symbol] |
| ATOM | 2315 | CG | ASP | A | 270 | 12.311 | 15.213 | 25.376 | 1.00 | 14.89 | [symbol] |
| ATOM | 2316 | OD1 | ASP | A | 270 | 11.368 | 15.718 | 26.018 | 1.00 | 14.96 | [symbol] |
| ATOM | 2317 | OD2 | ASP | A | 270 | 12.061 | 14.665 | 24.267 | 1.00 | 17.09 | [symbol] |
| ATOM | 2318 | C | ASP | A | 270 | 12.755 | 16.571 | 27.958 | 1.00 | 14.07 | [symbol] |
| ATOM | 2319 | O | ASP | A | 270 | 11.871 | 16.357 | 28.794 | 1.00 | 14.20 | [symbol] |
| ATOM | 2320 | N | GLU | A | 271 | 12.948 | 17.763 | 27.416 | 1.00 | 14.31 | [symbol] |
| ATOM | 2322 | CA | GLU | A | 271 | 12.154 | 18.915 | 27.816 | 1.00 | 14.56 | [symbol] |
| ATOM | 2324 | CB | GLU | A | 271 | 12.632 | 20.138 | 27.034 | 1.00 | 15.14 | [symbol] |
| ATOM | 2327 | CG | GLU | A | 271 | 12.577 | 21.432 | 27.821 | 1.00 | 16.67 | [symbol] |
| ATOM | 2330 | CD | GLU | A | 271 | 13.173 | 22.603 | 27.053 | 1.00 | 18.56 | [symbol] |
| ATOM | 2331 | OE1 | GLU | A | 271 | 13.233 | 22.537 | 25.801 | 1.00 | 19.69 | [symbol] |
| ATOM | 2332 | OE2 | GLU | A | 271 | 13.584 | 23.591 | 27.702 | 1.00 | 19.35 | [symbol] |
| ATOM | 2333 | C | GLU | A | 271 | 10.656 | 18.705 | 27.566 | 1.00 | 14.24 | [symbol] |
| ATOM | 2334 | O | GLU | A | 271 | 9.811 | 19.220 | 28.306 | 1.00 | 14.58 | [symbol] |
| ATOM | 2335 | N | GLU | A | 272 | 10.339 | 17.938 | 26.530 | 1.00 | 13.41 | [symbol] |
| ATOM | 2337 | CA | GLU | A | 272 | 8.964 | 17.740 | 26.095 | 1.00 | 12.91 | [symbol] |
| ATOM | 2339 | CB | GLU | A | 272 | 8.919 | 17.825 | 24.564 | 1.00 | 13.45 | [symbol] |
| ATOM | 2342 | CG | GLU | A | 272 | 7.646 | 17.287 | 23.930 | 1.00 | 15.09 | [symbol] |
| ATOM | 2345 | CD | GLU | A | 272 | 6.425 | 18.139 | 24.229 | 1.00 | 17.47 | [symbol] |
| ATOM | 2346 | OE1 | GLU | A | 272 | 6.010 | 18.937 | 23.354 | 1.00 | 17.98 | [symbol] |
| ATOM | 2347 | OE2 | GLU | A | 272 | 5.871 | 17.995 | 25.343 | 1.00 | 19.19 | [symbol] |
| ATOM | 2348 | C | GLU | A | 272 | 8.329 | 16.421 | 26.530 | 1.00 | 11.55 | [symbol] |
| ATOM | 2349 | O | GLU | A | 272 | 7.108 | 16.284 | 26.498 | 1.00 | 11.51 | [symbol] |
| ATOM | 2350 | N | THR | A | 273 | 9.134 | 15.452 | 26.944 | 1.00 | 9.75 | [symbol] |
| ATOM | 2352 | CA | THR | A | 273 | 8.585 | 14.129 | 27.218 | 1.00 | 8.69 | [symbol] |
| ATOM | 2354 | CB | THR | A | 273 | 9.075 | 13.153 | 26.159 | 1.00 | 8.76 | [symbol] |
| ATOM | 2356 | OG1 | TER | A | 273 | 9.047 | 13.781 | 24.869 | 1.00 | 9.12 | [symbol] |
| ATOM | 2358 | CG2 | THR | A | 273 | 8.126 | 11.978 | 26.041 | 1.00 | 9.77 | [symbol] |
| ATOM | 2362 | C | THR | A | 273 | 8.942 | 13.528 | 28.565 | 1.00 | 7.33 | [symbol] |
| ATOM | 2363 | O | THR | A | 273 | 10.073 | 13.621 | 29.005 | 1.00 | 7.19 | [symbol] |
| ATOM | 2364 | N | VAL | A | 274 | 7.953 | 12.863 | 29.151 | 1.00 | 6.08 | [symbol] |
| ATOM | 2366 | CA | VAL | A | 274 | 8.077 | 12.124 | 30.390 | 1.00 | 5.63 | [symbol] |
| ATOM | 2368 | CB | VAL | A | 274 | 7.038 | 12.603 | 31.427 | 1.00 | 5.35 | [symbol] |
| ATOM | 2370 | CG1 | VAL | A | 274 | 7.064 | 11.752 | 32.668 | 1.00 | 5.23 | [symbol] |
| ATOM | 2374 | CG2 | VAL | A | 274 | 7.260 | 14.059 | 31.782 | 1.00 | 6.17 | [symbol] |
| ATOM | 2378 | C | VAL | A | 274 | 7.804 | 10.658 | 30.066 | 1.00 | 5.28 | [symbol] |
| ATOM | 2379 | O | VAL | A | 274 | 6.843 | 10.333 | 29.349 | 1.00 | 4.93 | [symbol] |
| ATOM | 2380 | N | ILE | A | 275 | 8.673 | 9.779 | 30.545 | 1.00 | 4.74 | [symbol] |
| ATOM | 2382 | CA | ILE | A | 275 | 8.492 | 8.345 | 30.395 | 1.00 | 5.25 | [symbol] |
| ATOM | 2384 | CB | ILE | A | 275 | 9.845 | 7.644 | 30.146 | 1.00 | 6.00 | [symbol] |
| ATOM | 2386 | CG1 | ILE | A | 275 | 10.254 | 7.791 | 28.685 | 1.00 | 7.09 | [symbol] |
| ATOM | 2389 | CD1 | ILE | A | 275 | 9.124 | 7.536 | 27.688 | 1.00 | 8.89 | [symbol] |
| ATOM | 2393 | CG2 | ILE | A | 275 | 9.778 | 6.160 | 30.517 | 1.00 | 6.24 | [symbol] |
| ATOM | 2397 | C | ILE | A | 275 | 7.843 | 7.847 | 31.682 | 1.00 | 5.16 | [symbol] |
| ATOM | 2398 | O | ILE | A | 275 | 8.244 | 8.252 | 32.778 | 1.00 | 4.51 | [symbol] |
| ATOM | 2399 | N | ASP | A | 276 | 6.821 | 7.003 | 31.550 | 1.00 | 4.93 | [symbol] |
| ATOM | 2401 | CA | ASP | A | 276 | 6.097 | 6.499 | 32.712 | 1.00 | 4.99 | [symbol] |
| ATOM | 2403 | CB | ASP | A | 276 | 4.781 | 7.268 | 32.855 | 1.00 | 5.23 | [symbol] |
| ATOM | 2406 | CG | ASP | A | 276 | 4.056 | 6.983 | 34.159 | 1.00 | 5.97 | [symbol] |
| ATOM | 2407 | OD1 | ASP | A | 276 | 4.721 | 6.607 | 35.154 | 1.00 | 7.19 | [symbol] |
| ATOM | 2408 | OD2 | ASP | A | 276 | 2.818 | 7.146 | 34.273 | 1.00 | 6.97 | [symbol] |
| ATOM | 2409 | C | ASP | A | 276 | 5.813 | 5.004 | 32.610 | 1.00 | 5.08 | [symbol] |
| ATOM | 2410 | O | ASP | A | 276 | 5.674 | 4.459 | 31.522 | 1.00 | 4.61 | [symbol] |
| ATOM | 2411 | N | VAL | A | 277 | 5.758 | 4.335 | 33.761 | 1.00 | 5.26 | [symbol] |
| ATOM | 2413 | CA | VAL | A | 277 | 5.382 | 2.928 | 33.811 | 1.00 | 5.55 | [symbol] |
| ATOM | 2415 | CB | VAL | A | 277 | 6.506 | 2.054 | 34.354 | 1.00 | 6.07 | [symbol] |
| ATOM | 2417 | CG1 | VAL | A | 277 | 6.110 | 0.589 | 34.321 | 1.00 | 6.33 | [symbol] |
| ATOM | 2421 | CG2 | VAL | A | 277 | 7.767 | 2.265 | 33.537 | 1.00 | 6.87 | [symbol] |
| ATOM | 2425 | C | VAL | A | 277 | 4.143 | 2.842 | 34.703 | 1.00 | 5.75 | [symbol] |
| ATOM | 2426 | O | VAL | A | 277 | 4.226 | 2.531 | 35.888 | 1.00 | 5.17 | [symbol] |
| ATOM | 2427 | N | PRO | A | 278 | 2.995 | 3.165 | 34.130 | 1.00 | 5.68 | [symbol] |
| ATOM | 2428 | CA | PRO | A | 278 | 1.717 | 3.192 | 34.842 | 1.00 | 6.20 | [symbol] |
| ATOM | 2430 | CB | PRO | A | 278 | 0.738 | 3.734 | 33.780 | 1.00 | 6.37 | [symbol] |
| ATOM | 2433 | CG | PRO | A | 278 | 1.493 | 3.937 | 32.561 | 1.00 | 6.28 | [symbol] |
| ATOM | 2436 | CD | PRO | A | 278 | 2.853 | 3.518 | 32.708 | 1.00 | 5.79 | [symbol] |
| ATOM | 2439 | C | PRO | A | 278 | 1.214 | 1.820 | 35.278 | 1.00 | 6.95 | [symbol] |
| ATOM | 2440 | O | PRO | A | 278 | 1.647 | 0.810 | 34.733 | 1.00 | 6.97 | [symbol] |
| ATOM | 2441 | N | GLU | A | 279 | 0.314 | 1.794 | 36.259 | 1.00 | 7.70 | [symbol] |
| ATOM | 2443 | CA | GLU | A | 279 | −0.341 | 0.559 | 36.691 | 1.00 | 8.29 | [symbol] |
| ATOM | 2445 | CB | GLU | A | 279 | −1.627 | 0.928 | 37.450 | 1.00 | 8.86 | [symbol] |
| ATOM | 2448 | CG | GLU | A | 279 | −1.592 | 0.943 | 38.965 | 1.00 | 10.55 | [symbol] |
| ATOM | 2451 | CD | GLU | A | 279 | −0.797 | −0.198 | 39.546 | 1.00 | 11.50 | [symbol] |
| ATOM | 2452 | OE1 | GLU | A | 279 | −0.096 | 0.024 | 40.564 | 1.00 | 12.98 | [symbol] |
| ATOM | 2453 | OE2 | GLU | A | 279 | −0.874 | −1.317 | 38.996 | 1.00 | 12.19 | [symbol] |
| ATOM | 2454 | C | GLU | A | 279 | −0.786 | −0.106 | 35.394 | 1.00 | 8.23 | [symbol] |
| ATOM | 2455 | O | GLU | A | 279 | −1.127 | 0.658 | 34.486 | 1.00 | 8.51 | [symbol] |
| ATOM | 2456 | N | PRO | A | 280 | −0.849 | −1.424 | 35.159 | 1.00 | 8.03 | [symbol] |
| ATOM | 2457 | CA | PRO | A | 280 | −0.438 | −2.620 | 35.910 | 1.00 | 7.65 | [symbol] |
| ATOM | 2459 | CB | PRO | A | 280 | −1.239 | −3.753 | 35.191 | 1.00 | 7.99 | [symbol] |
| ATOM | 2462 | CG | PRO | A | 280 | −1.963 | −3.141 | 34.091 | 1.00 | 8.12 | [symbol] |
| ATOM | 2465 | CD | PRO | A | 280 | −1.524 | −1.783 | 33.914 | 1.00 | 8.30 | [symbol] |
| ATOM | 2468 | C | PRO | A | 280 | 1.043 | −2.900 | 35.695 | 1.00 | 6.68 | [symbol] |
| ATOM | 2469 | O | PRO | A | 280 | 1.691 | −3.577 | 36.482 | 1.00 | 7.93 | [symbol] |
| ATOM | 2470 | N | TYR | A | 281 | 1.575 | −2.321 | 34.628 | 1.00 | 5.83 | [symbol] |
| ATOM | 2472 | CA | TYR | A | 281 | 2.948 | −2.492 | 34.168 | 1.00 | 4.98 | [symbol] |
| ATOM | 2474 | CB | TYR | A | 281 | 3.143 | −1.626 | 32.932 | 1.00 | 4.93 | [symbol] |
| ATOM | 2477 | CG | TYR | A | 281 | 1.998 | −1.853 | 31.958 | 1.00 | 5.13 | [symbol] |
| ATOM | 2478 | CD1 | TYR | A | 281 | 1.142 | −0.824 | 31.599 | 1.00 | 6.00 | [symbol] |
| ATOM | 2480 | CE1 | TYR | A | 281 | 0.082 | −1.048 | 30.733 | 1.00 | 6.35 | [symbol] |
| ATOM | 2482 | CZ | TYR | A | 281 | −0.126 | −2.314 | 30.231 | 1.00 | 6.60 | [symbol] |
| ATOM | 2483 | OH | TYR | A | 281 | −1.168 | −2.555 | 29.374 | 1.00 | 7.98 | [symbol] |
| ATOM | 2485 | CE2 | TYR | A | 281 | 0.697 | −3.351 | 30.583 | 1.00 | 6.56 | [symbol] |
| ATOM | 2487 | CD2 | TYR | A | 281 | 1.748 | −3.119 | 31.445 | 1.00 | 5.49 | [symbol] |
| ATOM | 2489 | C | TYR | A | 281 | 4.061 | −2.236 | 35.181 | 1.00 | 4.34 | [symbol] |
| ATOM | 2490 | O | TYR | A | 281 | 5.201 | −2.564 | 34.898 | 1.00 | 4.10 | [symbol] |
| ATOM | 2491 | N | ASN | A | 282 | 3.744 | −1.655 | 36.335 | 1.00 | 3.78 | [symbol] |
| ATOM | 2493 | CA | ASN | A | 282 | 4.756 | −1.474 | 37.377 | 1.00 | 3.97 | [symbol] |
| ATOM | 2495 | CB | ASN | A | 282 | 4.402 | −0.340 | 38.344 | 1.00 | 4.11 | [symbol] |
| ATOM | 2498 | CG | ASN | A | 282 | 3.066 | −0.537 | 39.012 | 1.00 | 5.03 | [symbol] |
| ATOM | 2499 | OD1 | ASN | A | 282 | 2.065 | −0.819 | 38.361 | 1.00 | 5.68 | [symbol] |
| ATOM | 2500 | ND2 | ASN | A | 282 | 3.043 | −0.379 | 40.325 | 1.00 | 5.45 | N |
| ATOM | 2503 | C | ASN | A | 282 | 4.962 | −2.793 | 38.130 | 1.00 | 3.55 | [symbol] |
| ATOM | 2504 | O | ASN | A | 282 | 5.766 | −2.876 | 39.063 | 1.00 | 3.50 | [symbol] |
| ATOM | 2505 | N | HIS | A | 283 | 4.218 | −3.820 | 37.729 | 1.00 | 3.14 | N |
| ATOM | 2507 | CA | HIS | A | 283 | 4.330 | −5.155 | 38.302 | 1.00 | 2.99 | [symbol] |
| ATOM | 2509 | CB | HIS | A | 283 | 2.958 | −5.680 | 38.729 | 1.00 | 3.26 | [symbol] |
| ATOM | 2512 | CG | HIS | A | 283 | 2.198 | −4.747 | 39.619 | 1.00 | 3.72 | [symbol] |
| ATOM | 2513 | ND1 | HIS | A | 283 | 2.212 | −4.855 | 40.997 | 1.00 | 5.84 | N |
| ATOM | 2515 | CE1 | HIS | A | 283 | 1.466 | −3.896 | 41.517 | 1.00 | 5.78 | [symbol] |
| ATOM | 2517 | NE2 | HIS | A | 283 | 0.967 | −3.175 | 40.527 | 1.00 | 5.04 | N |
| ATOM | 2519 | CD2 | HIS | A | 283 | 1.427 | −3.673 | 39.336 | 1.00 | 4.01 | [symbol] |
| ATOM | 2521 | C | HIS | A | 283 | 4.959 | −6.077 | 37.250 | 1.00 | 3.18 | [symbol] |
| ATOM | 2522 | O | HIS | A | 283 | 4.542 | −6.077 | 36.083 | 1.00 | 2.66 | [symbol] |
| ATOM | 2523 | N | VAL | A | 284 | 5.962 | −6.852 | 37.664 | 1.00 | 3.38 | N |
| ATOM | 2525 | CA | VAL | A | 284 | 6.691 | −7.754 | 36.772 | 1.00 | 3.93 | [symbol] |
| ATOM | 2527 | CB | VAL | A | 284 | 7.902 | −8.397 | 37.522 | 1.00 | 4.61 | [symbol] |
| ATOM | 2529 | CG1 | VAL | A | 284 | 8.480 | −9.561 | 36.733 | 1.00 | 6.36 | [symbol] |
| ATOM | 2533 | CG2 | VAL | A | 284 | 8.951 | −7.367 | 37.765 | 1.00 | 5.28 | [symbol] |
| ATOM | 2537 | C | VAL | A | 284 | 5.793 | −8.835 | 36.177 | 1.00 | 3.60 | [symbol] |
| ATOM | 2538 | O | VAL | A | 284 | 6.077 | −9.374 | 35.102 | 1.00 | 3.80 | [symbol] |
| ATOM | 2539 | N | SER | A | 285 | 4.690 | −9.129 | 36.857 | 1.00 | 3.21 | N |
| ATOM | 2541 | CA | SER | A | 285 | 3.729 | −10.112 | 36.374 | 1.00 | 3.59 | [symbol] |
| ATOM | 2543 | CB | SER | A | 285 | 2.665 | −10.381 | 37.441 | 1.00 | 3.57 | [symbol] |
| ATOM | 2546 | OG | SER | A | 285 | 2.080 | −9.175 | 37.898 | 1.00 | 3.13 | [symbol] |
| ATOM | 2548 | C | SER | A | 285 | 3.053 | −9.624 | 35.091 | 1.00 | 3.14 | [symbol] |
| ATOM | 2549 | O | SER | A | 285 | 2.489 | −10.423 | 34.338 | 1.00 | 4.00 | [symbol] |
| ATOM | 2550 | N | LYS | A | 286 | 3.093 | −8.313 | 34.853 | 1.00 | 3.10 | N |
| ATOM | 2552 | CA | LYS | A | 286 | 2.439 | −7.702 | 33.678 | 1.00 | 3.42 | [symbol] |
| ATOM | 2554 | CB | LYS | A | 286 | 1.438 | −6.633 | 34.123 | 1.00 | 3.77 | [symbol] |
| ATOM | 2557 | CG | LYS | A | 286 | 0.123 | −7.194 | 34.685 | 1.00 | 6.06 | [symbol] |
| ATOM | 2560 | CD | LYS | A | 286 | −0.724 | −7.896 | 33.666 | 1.00 | 8.22 | [symbol] |
| ATOM | 2563 | CE | LYS | A | 286 | −1.925 | −8.600 | 34.311 | 1.00 | 9.47 | [symbol] |
| ATOM | 2566 | NZ | LYS | A | 286 | −2.793 | −9.261 | 33.296 | 1.00 | 11.77 | [symbol] |
| ATOM | 2570 | C | LYS | A | 286 | 3.405 | −7.077 | 32.670 | 1.00 | 3.19 | [symbol] |
| ATOM | 2571 | O | LYS | A | 286 | 3.067 | −6.937 | 31.484 | 1.00 | 3.19 | [symbol] |
| ATOM | 2572 | N | TYR | A | 287 | 4.591 | −6.686 | 33.135 | 1.00 | 3.08 | [symbol] |
| ATOM | 2574 | CA | TYR | A | 287 | 5.581 | −6.043 | 32.283 | 1.00 | 2.92 | [symbol] |
| ATOM | 2576 | CB | TYR | A | 287 | 5.327 | −4.534 | 32.227 | 1.00 | 3.01 | [symbol] |
| ATOM | 2579 | CG | TYR | A | 287 | 6.346 | −3.785 | 31.403 | 1.00 | 2.82 | [symbol] |
| ATOM | 2580 | CD1 | TYR | A | 287 | 6.227 | −3.716 | 30.020 | 1.00 | 2.65 | [symbol] |
| ATOM | 2582 | CE1 | TYR | A | 287 | 7.154 | −3.039 | 29.258 | 1.00 | 3.46 | [symbol] |
| ATOM | 2584 | CZ | TYR | A | 287 | 8.239 | −2.428 | 29.883 | 1.00 | 3.19 | [symbol] |
| ATOM | 2585 | OH | TYR | A | 287 | 9.179 | −1.762 | 29.138 | 1.00 | 3.19 | [symbol] |
| ATOM | 2587 | CE2 | TYR | A | 287 | 8.375 | −2.474 | 31.262 | 1.00 | 3.12 | [symbol] |
| ATOM | 2589 | CD2 | TYR | A | 287 | 7.438 | −3.153 | 32.008 | 1.00 | 2.85 | [symbol] |
| ATOM | 2591 | C | TYR | A | 287 | 6.994 | −6.291 | 32.781 | 1.00 | 3.16 | [symbol] |
| ATOM | 2592 | O | TYR | A | 287 | 7.328 | −5.923 | 33.904 | 1.00 | 2.77 | [symbol] |
| ATOM | 2593 | N | CYS | A | 288 | 7.825 | −6.899 | 31.943 | 1.00 | 3.14 | [symbol] |
| ATOM | 2595 | CA | CYS | A | 288 | 9.210 | −7.146 | 32.319 | 1.00 | 3.38 | [symbol] |
| ATOM | 2597 | CB | CYS | A | 288 | 9.379 | −8.530 | 32.957 | 1.00 | 3.55 | [symbol] |
| ATOM | 2600 | SG | CYS | A | 288 | 9.045 | −9.938 | 31.904 | 1.00 | 4.82 | [symbol] |
| ATOM | 2601 | C | CYS | A | 288 | 10.167 | −6.940 | 31.143 | 1.00 | 3.21 | [symbol] |
| ATOM | 2602 | O | CYS | A | 288 | 11.324 | −7.371 | 31.199 | 1.00 | 3.03 | [symbol] |
| ATOM | 2603 | N | ALA | A | 289 | 9.694 | −6.258 | 30.100 | 1.00 | 2.71 | [symbol] |
| ATOM | 2605 | CA | ALA | A | 289 | 10.523 | −5.935 | 28.941 | 1.00 | 2.80 | [symbol] |
| ATOM | 2607 | CB | ALA | A | 289 | 9.680 | −5.275 | 27.818 | 1.00 | 2.83 | [symbol] |
| ATOM | 2611 | C | ALA | A | 289 | 11.691 | −5.033 | 29.332 | 1.00 | 3.28 | [symbol] |
| ATOM | 2612 | O | ALA | A | 289 | 12.757 | −5.100 | 28.732 | 1.00 | 4.23 | [symbol] |
| ATOM | 2613 | N | SER | A | 290 | 11.496 | −4.204 | 30.354 | 1.00 | 3.41 | [symbol] |
| ATOM | 2615 | CA | SER | A | 290 | 12.558 | −3.353 | 30.882 | 1.00 | 3.17 | [symbol] |
| ATOM | 2617 | CB | SER | A | 290 | 12.535 | −1.959 | 30.266 | 1.00 | 3.30 | [symbol] |
| ATOM | 2620 | OG | SER | A | 290 | 11.322 | −1.293 | 30.525 | 1.00 | 2.87 | [symbol] |
| ATOM | 2622 | C | SER | A | 290 | 12.317 | −3.286 | 32.377 | 1.00 | 3.07 | [symbol] |
| ATOM | 2623 | O | SER | A | 290 | 11.195 | −3.478 | 32.823 | 1.00 | 2.76 | [symbol] |
| ATOM | 2624 | N | LEU | A | 291 | 13.349 | −2.993 | 33.159 | 1.00 | 2.85 | [symbol] |
| ATOM | 2626 | CA | LEU | A | 291 | 13.206 | −3.042 | 34.601 | 1.00 | 2.89 | [symbol] |
| ATOM | 2628 | CB | LEU | A | 291 | 13.931 | −4.286 | 35.143 | 1.00 | 2.91 | [symbol] |
| ATOM | 2631 | CG | LEU | A | 291 | 13.374 | −5.624 | 34.631 | 1.00 | 2.88 | [symbol] |
| ATOM | 2633 | CD1 | LEU | A | 291 | 14.284 | −6.783 | 34.977 | 1.00 | 3.99 | [symbol] |
| ATOM | 2637 | CD2 | LEU | A | 291 | 11.955 | −5.886 | 35.147 | 1.00 | 2.41 | [symbol] |
| ATOM | 2641 | C | LEU | A | 291 | 13.712 | −1.810 | 35.332 | 1.00 | 2.94 | [symbol] |
| ATOM | 2642 | O | LEU | A | 291 | 13.742 | −1.805 | 36.562 | 1.00 | 2.78 | [symbol] |
| ATOM | 2643 | N | GLY | A | 292 | 14.073 | −0.766 | 34.587 | 1.00 | 2.64 | [symbol] |
| ATOM | 2645 | CA | GLY | A | 292 | 14.630 | 0.449 | 35.169 | 1.00 | 2.69 | [symbol] |
| ATOM | 2648 | C | GLY | A | 292 | 13.795 | 1.106 | 36.250 | 1.00 | 2.65 | [symbol] |
| ATOM | 2649 | O | GLY | A | 292 | 14.336 | 1.707 | 37.187 | 1.00 | 2.68 | [symbol] |
| ATOM | 2650 | N | HIS | A | 293 | 12.478 | 0.991 | 36.126 | 1.00 | 2.35 | [symbol] |
| ATOM | 2652 | CA | HIS | A | 293 | 11.550 | 1.619 | 37.055 | 1.00 | 2.26 | [symbol] |
| ATOM | 2654 | CB | HIS | A | 293 | 10.148 | 1.602 | 36.437 | 1.00 | 2.29 | [symbol] |
| ATOM | 2657 | CG | HIS | A | 293 | 9.712 | 0.229 | 36.055 | 1.00 | 2.25 | [symbol] |
| ATOM | 2658 | ND1 | HIS | A | 293 | 10.181 | −0.403 | 34.926 | 1.00 | 2.25 | [symbol] |
| ATOM | 2660 | CE1 | HIS | A | 293 | 9.648 | −1.609 | 34.848 | 1.00 | 2.36 | [symbol] |
| ATOM | 2662 | NE2 | HIS | A | 293 | 8.885 | −1.797 | 35.910 | 1.00 | 2.79 | [symbol] |
| ATOM | 2664 | CD2 | HIS | A | 293 | 8.918 | −0.666 | 36.690 | 1.00 | 2.34 | [symbol] |
| ATOM | 2666 | C | HIS | A | 293 | 11.504 | 0.929 | 38.421 | 1.00 | 2.56 | [symbol] |
| ATOM | 2667 | O | HIS | A | 293 | 10.796 | 1.395 | 39.313 | 1.00 | 2.98 | [symbol] |
| ATOM | 2668 | N | LYS | A | 294 | 12.240 | −0.172 | 38.582 | 1.00 | 2.27 | [symbol] |
| ATOM | 2670 | CA | LYS | A | 294 | 12.264 | −0.919 | 39.839 | 1.00 | 2.63 | [symbol] |
| ATOM | 2672 | CB | LYS | A | 294 | 12.376 | −2.421 | 39.565 | 1.00 | 2.92 | [symbol] |
| ATOM | 2675 | CG | LYS | A | 294 | 11.067 | −3.031 | 39.101 | 1.00 | 3.32 | [symbol] |
| ATOM | 2678 | CD | LYS | A | 294 | 10.086 | −3.141 | 40.258 | 1.00 | 4.06 | [symbol] |
| ATOM | 2681 | CE | LYS | A | 294 | 8.697 | −3.522 | 39.807 | 1.00 | 4.08 | [symbol] |
| ATOM | 2684 | NZ | LYS | A | 294 | 7.734 | −3.623 | 40.939 | 1.00 | 4.41 | [symbol] |
| ATOM | 2688 | C | LYS | A | 294 | 13.399 | −0.470 | 40.746 | 1.00 | 2.51 | [symbol] |
| ATOM | 2689 | O | LYS | A | 294 | 13.471 | −0.868 | 41.922 | 1.00 | 2.44 | [symbol] |
| ATOM | 2690 | N | ALA | A | 295 | 14.293 | 0.366 | 40.230 | 1.00 | 2.47 | [symbol] |
| ATOM | 2692 | CA | ALA | A | 295 | 15.431 | 0.814 | 41.054 | 1.00 | 2.22 | [symbol] |
| ATOM | 2694 | CB | ALA | A | 295 | 16.548 | 1.337 | 40.185 | 1.00 | 2.36 | [symbol] |
| ATOM | 2698 | C | ALA | A | 295 | 14.970 | 1.893 | 42.034 | 1.00 | 2.25 | [symbol] |
| ATOM | 2699 | O | ALA | A | 295 | 14.358 | 2.889 | 41.637 | 1.00 | 2.76 | [symbol] |
| ATOM | 2700 | N | ASN | A | 296 | 15.253 | 1.682 | 43.313 | 1.00 | 2.13 | [symbol] |
| ATOM | 2702 | CA | ASN | A | 296 | 14.860 | 2.615 | 44.361 | 1.00 | 2.29 | [symbol] |
| ATOM | 2704 | CB | ASN | A | 296 | 14.725 | 1.873 | 45.678 | 1.00 | 2.09 | [symbol] |
| ATOM | 2707 | CG | ASN | A | 296 | 13.486 | 1.028 | 45.711 | 1.00 | 2.28 | [symbol] |
| ATOM | 2708 | OD1 | ASN | A | 296 | 12.422 | 1.486 | 45.279 | 1.00 | 2.81 | [symbol] |
| ATOM | 2709 | ND2 | ASN | A | 296 | 13.615 | −0.221 | 46.121 | 1.00 | 2.55 | [symbol] |
| ATOM | 2712 | C | ASN | A | 296 | 15.786 | 3.816 | 44.496 | 1.00 | 2.15 | [symbol] |
| ATOM | 2713 | O | ASN | A | 296 | 16.853 | 3.860 | 43.893 | 1.00 | 2.45 | [symbol] |
| ATOM | 2714 | N | HIS | A | 297 | 15.344 | 4.798 | 45.271 | 1.00 | 2.20 | [symbol] |
| ATOM | 2716 | CA | HIS | A | 297 | 16.080 | 6.034 | 45.450 | 1.00 | 2.13 | [symbol] |
| ATOM | 2718 | CB | HIS | A | 297 | 15.121 | 7.173 | 45.807 | 1.00 | 2.21 | [symbol] |
| ATOM | 2721 | CG | HIS | A | 297 | 15.829 | 8.394 | 46.302 | 1.00 | 2.34 | [symbol] |
| ATOM | 2722 | ND1 | HIS | A | 297 | 16.462 | 9.281 | 45.458 | 1.00 | 2.56 | [symbol] |
| ATOM | 2724 | CE1 | HIS | A | 297 | 17.037 | 10.232 | 46.173 | 1.00 | 2.82 | [symbol] |
| ATOM | 2726 | NE2 | HIS | A | 297 | 16.815 | 9.984 | 47.451 | 1.00 | 2.94 | [symbol] |
| ATOM | 2728 | CD2 | HIS | A | 297 | 16.066 | 8.838 | 47.561 | 1.00 | 2.18 | [symbol] |
| ATOM | 2730 | C | HIS | A | 297 | 17.130 | 5.980 | 46.554 | 1.00 | 2.15 | [symbol] |
| ATOM | 2731 | O | HIS | A | 297 | 16.948 | 5.305 | 47.551 | 1.00 | 2.33 | [symbol] |
| ATOM | 2732 | N | SER | A | 298 | 18.216 | 6.721 | 46.364 | 1.00 | 2.19 | [symbol] |
| ATOM | 2734 | CA | SER | A | 298 | 19.215 | 6.914 | 47.405 | 1.00 | 2.49 | [symbol] |
| ATOM | 2736 | CB | SER | A | 298 | 20.243 | 5.783 | 47.431 | 1.00 | 2.80 | [symbol] |
| ATOM | 2739 | OG | SER | A | 298 | 21.287 | 6.091 | 48.325 | 1.00 | 2.20 | [symbol] |
| ATOM | 2741 | C | SER | A | 298 | 19.911 | 8.241 | 47.136 | 1.00 | 2.39 | [symbol] |
| ATOM | 2742 | O | SER | A | 298 | 20.109 | 8.615 | 45.982 | 1.00 | 2.37 | [symbol] |
| ATOM | 2743 | N | PHE | A | 299 | 20.277 | 8.943 | 48.207 | 1.00 | 2.46 | [symbol] |
| ATOM | 2745 | CA | PHE | A | 299 | 21.037 | 10.186 | 48.092 | 1.00 | 2.65 | [symbol] |
| ATOM | 2747 | CB | PHE | A | 299 | 20.745 | 11.104 | 49.279 | 1.00 | 2.42 | [symbol] |
| ATOM | 2750 | CG | PHE | A | 299 | 19.338 | 11.659 | 49.286 | 1.00 | 2.34 | [symbol] |
| ATOM | 2751 | CD1 | PHE | A | 299 | 18.416 | 11.245 | 50.231 | 1.00 | 2.37 | [symbol] |
| ATOM | 2753 | CE1 | PHE | A | 299 | 17.133 | 11.763 | 50.240 | 1.00 | 2.97 | [symbol] |
| ATOM | 2755 | CZ | PHE | A | 299 | 16.752 | 12.697 | 49.297 | 1.00 | 2.88 | [symbol] |
| ATOM | 2757 | CE2 | PHE | A | 299 | 17.657 | 13.115 | 48.345 | 1.00 | 3.21 | [symbol] |
| ATOM | 2759 | CD2 | PHE | A | 299 | 18.945 | 12.599 | 48.341 | 1.00 | 2.74 | [symbol] |
| ATOM | 2761 | C | PHE | A | 299 | 22.543 | 9.873 | 47.963 | 1.00 | 2.90 | [symbol] |
| ATOM | 2762 | O | PHE | A | 299 | 23.370 | 10.764 | 47.722 | 1.00 | 3.44 | [symbol] |
| ATOM | 2763 | N | THR | A | 300 | 22.901 | 8.605 | 48.151 | 1.00 | 2.84 | [symbol] |
| ATOM | 2765 | CA | THR | A | 300 | 24.260 | 8.135 | 47.903 | 1.00 | 2.81 | [symbol] |
| ATOM | 2767 | CB | THR | A | 300 | 24.960 | 7.752 | 49.219 | 1.00 | 3.19 | [symbol] |
| ATOM | 2769 | OG1 | THR | A | 300 | 24.282 | 6.645 | 49.839 | 1.00 | 3.63 | [symbol] |
| ATOM | 2771 | CG2 | THR | A | 300 | 24.845 | 8.865 | 50.233 | 1.00 | 3.94 | [symbol] |
| ATOM | 2775 | C | THR | A | 300 | 24.177 | 6.940 | 46.941 | 1.00 | 2.44 | [symbol] |
| ATOM | 2776 | O | THR | A | 300 | 24.554 | 5.827 | 47.265 | 1.00 | 2.31 | [symbol] |
| ATOM | 2777 | N | PRO | A | 301 | 23.657 | 7.180 | 45.737 | 1.00 | 2.43 | [symbol] |
| ATOM | 2778 | CA | PRO | A | 301 | 23.378 | 6.110 | 44.781 | 1.00 | 2.16 | [symbol] |
| ATOM | 2780 | CB | PRO | A | 301 | 22.571 | 6.820 | 43.687 | 1.00 | 2.48 | [symbol] |
| ATOM | 2783 | CG | PRO | A | 301 | 22.859 | 8.261 | 43.848 | 1.00 | 2.59 | [symbol] |
| ATOM | 2786 | CD | PRO | A | 301 | 23.355 | 8.504 | 45.187 | 1.00 | 2.30 | [symbol] |
| ATOM | 2789 | C | PRO | A | 301 | 24.612 | 5.508 | 44.146 | 1.00 | 2.19 | [symbol] |
| ATOM | 2790 | O | PRO | A | 301 | 25.667 | 6.142 | 44.118 | 1.00 | 2.11 | [symbol] |
| ATOM | 2791 | N | ASN | A | 302 | 24.473 | 4.280 | 43.663 | 1.00 | 2.08 | [symbol] |
| ATOM | 2793 | CA | ASN | A | 302 | 25.544 | 3.631 | 42.923 | 1.00 | 2.30 | [symbol] |
| ATOM | 2795 | CB | ASN | A | 302 | 25.760 | 2.176 | 43.368 | 1.00 | 2.54 | [symbol] |
| ATOM | 2798 | CG | ASN | A | 302 | 24.503 | 1.347 | 43.345 | 1.00 | 2.93 | [symbol] |
| ATOM | 2799 | OD1 | ASN | A | 302 | 23.527 | 1.646 | 42.642 | 1.00 | 2.27 | [symbol] |
| ATOM | 2800 | ND2 | ASN | A | 302 | 24.524 | 0.276 | 44.126 | 1.00 | 2.69 | [symbol] |
| ATOM | 2803 | C | ASN | A | 302 | 25.305 | 3.719 | 41.414 | 1.00 | 2.18 | [symbol] |
| ATOM | 2804 | O | ASN | A | 302 | 26.127 | 3.272 | 40.629 | 1.00 | 2.31 | [symbol] |
| ATOM | 2805 | N | CYS | A | 303 | 24.182 | 4.317 | 41.022 | 1.00 | 2.27 | [symbol] |
| ATOM | 2807 | CA | CYS | A | 303 | 23.833 | 4.451 | 39.602 | 1.00 | 2.49 | [symbol] |
| ATOM | 2809 | CB | CYS | A | 303 | 22.792 | 3.396 | 39.210 | 1.00 | 2.78 | [symbol] |
| ATOM | 2812 | SG | CYS | A | 303 | 23.339 | 1.693 | 39.246 | 1.00 | 3.80 | [symbol] |
| ATOM | 2813 | C | CYS | A | 303 | 23.254 | 5.829 | 39.291 | 1.00 | 2.73 | [symbol] |
| ATOM | 2814 | O | CYS | A | 303 | 22.880 | 6.575 | 40.195 | 1.00 | 2.38 | [symbol] |
| ATOM | 2815 | N | ILE | A | 304 | 23.175 | 6.144 | 37.997 | 1.00 | 3.20 | [symbol] |
| ATOM | 2817 | CA | ILE | A | 304 | 22.551 | 7.362 | 37.507 | 1.00 | 4.01 | [symbol] |
| ATOM | 2819 | CB | ILE | A | 304 | 23.589 | 8.352 | 36.962 | 1.00 | 4.63 | [symbol] |
| ATOM | 2821 | CG1 | ILE | A | 304 | 22.900 | 9.662 | 36.546 | 1.00 | 6.24 | [symbol] |
| ATOM | 2824 | CD1 | ILE | A | 304 | 23.714 | 10.929 | 36.824 | 1.00 | 7.94 | [symbol] |
| ATOM | 2828 | CG2 | ILE | A | 304 | 24.294 | 7.762 | 35.735 | 1.00 | 3.91 | [symbol] |
| ATOM | 2832 | C | ILE | A | 304 | 21.615 | 7.005 | 36.356 | 1.00 | 3.90 | [symbol] |
| ATOM | 2833 | O | ILE | A | 304 | 21.779 | 5.967 | 35.706 | 1.00 | 3.61 | [symbol] |
| ATOM | 2834 | N | TYR | A | 305 | 20.620 | 7.851 | 36.115 | 1.00 | 4.27 | [symbol] |
| ATOM | 2836 | CA | TYR | A | 305 | 19.811 | 7.719 | 34.905 | 1.00 | 4.17 | [symbol] |
| ATOM | 2838 | CB | TYR | A | 305 | 18.446 | 8.396 | 35.049 | 1.00 | 4.27 | [symbol] |
| ATOM | 2841 | CG | TYR | A | 305 | 17.518 | 7.784 | 36.075 | 1.00 | 4.35 | [symbol] |
| ATOM | 2842 | CD1 | TYR | A | 305 | 16.969 | 8.561 | 37.079 | 1.00 | 3.85 | [symbol] |
| ATOM | 2844 | CE1 | TYR | A | 305 | 16.088 | 8.031 | 37.989 | 1.00 | 3.52 | [symbol] |
| ATOM | 2846 | CZ | TYR | A | 305 | 15.742 | 6.705 | 37.916 | 1.00 | 2.97 | [symbol] |
| ATOM | 2847 | OH | TYR | A | 305 | 14.899 | 6.178 | 38.864 | 1.00 | 3.40 | [symbol] |
| ATOM | 2849 | CE2 | TYR | A | 305 | 16.272 | 5.905 | 36.938 | 1.00 | 3.18 | [symbol] |
| ATOM | 2851 | CD2 | TYR | A | 305 | 17.145 | 6.445 | 36.009 | 1.00 | 3.93 | [symbol] |
| ATOM | 2853 | C | TYR | A | 305 | 20.594 | 8.424 | 33.793 | 1.00 | 4.33 | [symbol] |
| ATOM | 2854 | O | TYR | A | 305 | 21.046 | 9.556 | 33.979 | 1.00 | 4.86 | [symbol] |
| ATOM | 2855 | N | ASP | A | 306 | 20.757 | 7.766 | 32.646 | 1.00 | 4.34 | [symbol] |
| ATOM | 2857 | CA | ASP | A | 306 | 21.513 | 8.308 | 31.529 | 1.00 | 4.40 | [symbol] |
| ATOM | 2859 | CB | ASP | A | 306 | 22.849 | 7.584 | 31.422 | 1.00 | 4.75 | [symbol] |
| ATOM | 2862 | CG | ASP | A | 306 | 23.936 | 8.435 | 30.780 | 1.00 | 7.01 | [symbol] |
| ATOM | 2863 | OD1 | ASP | A | 306 | 25.078 | 7.942 | 30.676 | 1.00 | 9.18 | [symbol] |
| ATOM | 2864 | OD2 | ASP | A | 306 | 23.753 | 9.599 | 30.359 | 1.00 | 8.97 | [symbol] |
| ATOM | 2865 | C | ASP | A | 306 | 20.724 | 8.051 | 30.246 | 1.00 | 4.09 | [symbol] |
| ATOM | 2866 | O | ASP | A | 306 | 19.871 | 7.178 | 30.213 | 1.00 | 4.07 | [symbol] |
| ATOM | 2867 | N | MET | A | 307 | 21.019 | 8.816 | 29.205 | 1.00 | 4.07 | [symbol] |
| ATOM | 2869 | CA | MET | A | 307 | 20.347 | 8.664 | 27.912 | 1.00 | 4.21 | [symbol] |
| ATOM | 2871 | CB | MET | A | 307 | 20.783 | 9.786 | 26.977 | 1.00 | 5.32 | [symbol] |
| ATOM | 2874 | CG | MET | A | 307 | 20.489 | 11.178 | 27.519 | 1.00 | 8.07 | [symbol] |
| ATOM | 2877 | SD | MET | A | 307 | 18.720 | 11.457 | 27.739 | 1.00 | 14.04 | [symbol] |
| ATOM | 2878 | CE | MET | A | 307 | 18.218 | 11.779 | 26.162 | 1.00 | 13.82 | [symbol] |
| ATOM | 2882 | C | MET | A | 307 | 20.701 | 7.330 | 27.284 | 1.00 | 3.48 | [symbol] |
| ATOM | 2883 | O | MET | A | 307 | 21.779 | 6.778 | 27.512 | 1.00 | 3.03 | [symbol] |
| ATOM | 2884 | N | PHE | A | 308 | 19.800 | 6.798 | 26.472 | 1.00 | 2.95 | [symbol] |
| ATOM | 2886 | CA | PHE | A | 308 | 20.089 | 5.560 | 25.787 | 1.00 | 2.68 | [symbol] |
| ATOM | 2888 | CB | PHE | A | 308 | 19.816 | 4.342 | 26.658 | 1.00 | 2.48 | [symbol] |
| ATOM | 2891 | CG | PHE | A | 308 | 20.525 | 3.109 | 26.175 | 1.00 | 2.58 | [symbol] |
| ATOM | 2892 | CD1 | PHE | A | 308 | 21.838 | 2.872 | 26.533 | 1.00 | 2.38 | [symbol] |
| ATOM | 2894 | CE1 | PHE | A | 308 | 22.506 | 1.749 | 26.081 | 1.00 | 2.34 | [symbol] |
| ATOM | 2896 | CZ | PHE | A | 308 | 21.861 | 0.859 | 25.233 | 1.00 | 2.21 | [symbol] |
| ATOM | 2898 | CE2 | PHE | A | 308 | 20.561 | 1.091 | 24.864 | 1.00 | 2.27 | [symbol] |
| ATOM | 2900 | CD2 | PHE | A | 308 | 19.899 | 2.213 | 25.323 | 1.00 | 2.69 | [symbol] |
| ATOM | 2902 | C | PHE | A | 308 | 19.247 | 5.463 | 24.556 | 1.00 | 2.59 | [symbol] |
| ATOM | 2903 | O | PHE | A | 308 | 18.077 | 5.819 | 24.590 | 1.00 | 2.93 | [symbol] |
| ATOM | 2904 | N | VAL | A | 309 | 19.846 | 4.997 | 23.470 | 1.00 | 2.55 | [symbol] |
| ATOM | 2906 | CA | VAL | A | 309 | 19.084 | 4.741 | 22.238 | 1.00 | 2.34 | [symbol] |
| ATOM | 2908 | CB | VAL | A | 309 | 19.779 | 5.286 | 20.996 | 1.00 | 2.39 | [symbol] |
| ATOM | 2910 | CG1 | VAL | A | 309 | 18.970 | 4.927 | 19.749 | 1.00 | 3.20 | [symbol] |
| ATOM | 2914 | CG2 | VAL | A | 309 | 19.947 | 6.783 | 21.101 | 1.00 | 2.94 | [symbol] |
| ATOM | 2918 | C | VAL | A | 309 | 18.929 | 3.234 | 22.085 | 1.00 | 2.29 | [symbol] |
| ATOM | 2919 | O | VAL | A | 309 | 19.908 | 2.537 | 21.784 | 1.00 | 2.81 | [symbol] |
| ATOM | 2920 | N | HIS | A | 310 | 17.708 | 2.735 | 22.288 | 1.00 | 2.05 | [symbol] |
| ATOM | 2922 | CA | HIS | A | 310 | 17.437 | 1.302 | 22.283 | 1.00 | 2.05 | [symbol] |
| ATOM | 2924 | CB | HIS | A | 310 | 16.515 | 0.970 | 23.451 | 1.00 | 2.06 | [symbol] |
| ATOM | 2927 | CG | HIS | A | 310 | 16.542 | −0.468 | 23.854 | 1.00 | 2.16 | [symbol] |
| ATOM | 2928 | ND1 | HIS | A | 310 | 15.910 | −1.456 | 23.133 | 1.00 | 2.24 | [symbol] |
| ATOM | 2930 | CE1 | HIS | A | 310 | 16.094 | −2.620 | 23.729 | 1.00 | 2.24 | [symbol] |
| ATOM | 2932 | NE2 | HIS | A | 310 | 16.841 | −2.424 | 24.804 | 1.00 | 2.07 | [symbol] |
| ATOM | 2934 | CD2 | HIS | A | 310 | 17.129 | −1.087 | 24.907 | 1.00 | 2.11 | [symbol] |
| ATOM | 2936 | C | HIS | A | 310 | 16.780 | 0.859 | 20.978 | 1.00 | 2.02 | [symbol] |
| ATOM | 2937 | O | HIS | A | 310 | 15.853 | 1.516 | 20.508 | 1.00 | 2.15 | [symbol] |
| ATOM | 2938 | N | PRO | A | 311 | 17.236 | −0.253 | 20.404 | 1.00 | 2.00 | [symbol] |
| ATOM | 2939 | CA | PRO | A | 311 | 16.729 | −0.708 | 19.113 | 1.00 | 2.03 | [symbol] |
| ATOM | 2941 | CB | PRO | A | 311 | 17.679 | −1.853 | 18.779 | 1.00 | 2.05 | [symbol] |
| ATOM | 2944 | CG | PRO | A | 311 | 18.002 | −2.396 | 20.074 | 1.00 | 2.00 | [symbol] |
| ATOM | 2947 | CD | PRO | A | 311 | 18.269 | −1.178 | 20.901 | 1.00 | 2.25 | [symbol] |
| ATOM | 2950 | C | PRO | A | 311 | 15.305 | −1.227 | 19.166 | 1.00 | 2.06 | [symbol] |
| ATOM | 2951 | O | PRO | A | 311 | 14.682 | −1.344 | 18.121 | 1.00 | 2.04 | [symbol] |
| ATOM | 2952 | N | ARG | A | 312 | 14.825 | −1.556 | 20.363 | 1.00 | 2.07 | [symbol] |
| ATOM | 2954 | CA | ARG | A | 312 | 13.457 | −2.019 | 20.544 | 1.00 | 2.05 | [symbol] |
| ATOM | 2956 | CB | ARG | A | 312 | 13.451 | −3.193 | 21.520 | 1.00 | 2.11 | [symbol] |
| ATOM | 2959 | CG | ARG | A | 312 | 12.081 | −3.729 | 21.869 | 1.00 | 2.61 | [symbol] |
| ATOM | 2962 | CD | ARG | A | 312 | 12.183 | −4.845 | 22.888 | 1.00 | 2.59 | [symbol] |
| ATOM | 2965 | NE | ARG | A | 312 | 10.909 | −5.399 | 23.321 | 1.00 | 2.54 | [symbol] |
| ATOM | 2967 | CZ | ARG | A | 312 | 10.809 | −6.309 | 24.281 | 1.00 | 2.35 | [symbol] |
| ATOM | 2968 | NH1 | ARG | A | 312 | 11.910 | −6.750 | 24.882 | 1.00 | 2.09 | [symbol] |
| ATOM | 2971 | NH2 | ARG | A | 312 | 9.633 | −6.785 | 24.651 | 1.00 | 2.21 | [symbol] |
| ATOM | 2974 | C | ARG | A | 312 | 12.555 | −0.906 | 21.079 | 1.00 | 2.01 | [symbol] |
| ATOM | 2975 | O | ARG | A | 312 | 11.441 | −0.707 | 20.585 | 1.00 | 2.03 | [symbol] |
| ATOM | 2976 | N | PHE | A | 313 | 13.048 | −0.180 | 22.082 | 1.00 | 2.06 | [symbol] |
| ATOM | 2978 | CA | PHE | A | 313 | 12.272 | 0.852 | 22.768 | 1.00 | 2.15 | [symbol] |
| ATOM | 2980 | CB | PHE | A | 313 | 12.646 | 0.908 | 24.252 | 1.00 | 2.09 | [symbol] |
| ATOM | 2983 | CG | PHE | A | 313 | 12.593 | −0.422 | 24.940 | 1.00 | 2.49 | [symbol] |
| ATOM | 2984 | CD1 | PHE | A | 313 | 13.706 | −0.932 | 25.595 | 1.00 | 2.43 | [symbol] |
| ATOM | 2986 | CE1 | PHE | A | 313 | 13.649 | −2.151 | 26.218 | 1.00 | 2.71 | [symbol] |
| ATOM | 2988 | CZ | PHE | A | 313 | 12.476 | −2.890 | 26.186 | 1.00 | 3.14 | [symbol] |
| ATOM | 2990 | CE2 | PHE | A | 313 | 11.374 | −2.388 | 25.543 | 1.00 | 3.93 | [symbol] |
| ATOM | 2992 | CD2 | PHE | A | 313 | 11.432 | −1.170 | 24.924 | 1.00 | 3.07 | [symbol] |
| ATOM | 2994 | C | PHE | A | 313 | 12.411 | 2.260 | 22.202 | 1.00 | 2.21 | [symbol] |
| ATOM | 2995 | O | PHE | A | 313 | 11.577 | 3.113 | 22.496 | 1.00 | 2.40 | [symbol] |
| ATOM | 2996 | N | GLY | A | 314 | 13.465 | 2.515 | 21.430 | 1.00 | 2.22 | [symbol] |
| ATOM | 2998 | CA | GLY | A | 314 | 13.703 | 3.846 | 20.898 | 1.00 | 2.38 | [symbol] |
| ATOM | 3001 | C | GLY | A | 314 | 14.504 | 4.673 | 21.888 | 1.00 | 2.42 | [symbol] |
| ATOM | 3002 | O | GLY | A | 314 | 15.170 | 4.117 | 22.771 | 1.00 | 2.50 | [symbol] |
| ATOM | 3003 | N | PRO | A | 315 | 14.474 | 5.995 | 21.752 | 1.00 | 2.39 | [symbol] |
| ATOM | 3004 | CA | PRO | A | 315 | 15.205 | 6.863 | 22.682 | 1.00 | 2.53 | [symbol] |
| ATOM | 3006 | CB | PRO | A | 315 | 15.125 | 8.244 | 22.017 | 1.00 | 2.53 | [symbol] |
| ATOM | 3009 | CG | PRO | A | 315 | 14.561 | 8.011 | 20.631 | 1.00 | 2.82 | [symbol] |
| ATOM | 3012 | CD | PRO | A | 315 | 13.766 | 6.768 | 20.718 | 1.00 | 2.60 | [symbol] |
| ATOM | 3015 | C | PRO | A | 315 | 14.553 | 6.856 | 24.064 | 1.00 | 2.29 | [symbol] |
| ATOM | 3016 | O | PRO | A | 315 | 13.414 | 7.277 | 24.218 | 1.00 | 2.54 | [symbol] |
| ATOM | 3017 | N | ILE | A | 316 | 15.279 | 6.371 | 25.065 | 1.00 | 2.65 | [symbol] |
| ATOM | 3019 | CA | ILE | A | 316 | 14.766 | 6.267 | 26.421 | 1.00 | 2.47 | [symbol] |
| ATOM | 3021 | CB | ILE | A | 316 | 14.346 | 4.822 | 26.706 | 1.00 | 2.35 | [symbol] |
| ATOM | 3023 | CG1 | ILE | A | 316 | 15.497 | 3.870 | 26.375 | 1.00 | 2.33 | [symbol] |
| ATOM | 3026 | CD1 | ILE | A | 316 | 15.293 | 2.446 | 26.877 | 1.00 | 2.49 | [symbol] |
| ATOM | 3030 | CG2 | ILE | A | 316 | 13.104 | 4.465 | 25.901 | 1.00 | 3.20 | [symbol] |
| ATOM | 3034 | C | ILE | A | 316 | 15.879 | 6.678 | 27.374 | 1.00 | 2.60 | [symbol] |
| ATOM | 3035 | O | ILE | A | 316 | 16.785 | 7.397 | 26.977 | 1.00 | 2.71 | [symbol] |
| ATOM | 3036 | N | LYS | A | 317 | 15.811 | 6.223 | 28.622 | 1.00 | 3.12 | [symbol] |
| ATOM | 3038 | CA | LYS | A | 317 | 16.918 | 6.422 | 29.560 | 1.00 | 3.53 | [symbol] |
| ATOM | 3040 | CB | LYS | A | 317 | 16.561 | 7.348 | 30.733 | 1.00 | 4.01 | [symbol] |
| ATOM | 3043 | CG | LYS | A | 317 | 16.618 | 8.849 | 30.330 | 1.00 | 5.08 | [symbol] |
| ATOM | 3046 | CD | LYS | A | 317 | 16.663 | 9.811 | 31.503 | 1.00 | 5.59 | [symbol] |
| ATOM | 3049 | CE | LYS | A | 317 | 16.792 | 11.261 | 31.003 | 1.00 | 5.93 | [symbol] |
| ATOM | 3052 | NZ | LYS | A | 317 | 16.702 | 12.281 | 32.080 | 1.00 | 5.69 | [symbol] |
| ATOM | 3056 | C | LYS | A | 317 | 17.334 | 5.033 | 30.034 | 1.00 | 3.26 | [symbol] |
| ATOM | 3057 | O | LYS | A | 317 | 16.600 | 4.056 | 29.832 | 1.00 | 3.14 | [symbol] |
| ATOM | 3058 | N | CYS | A | 318 | 18.520 | 4.937 | 30.615 | 1.00 | 3.10 | [symbol] |
| ATOM | 3060 | CA | CYS | A | 318 | 19.029 | 3.676 | 31.146 | 1.00 | 2.84 | [symbol] |
| ATOM | 3062 | CB | CYS | A | 318 | 20.146 | 3.121 | 30.260 | 1.00 | 2.61 | [symbol] |
| ATOM | 3065 | SG | CYS | A | 318 | 21.710 | 4.019 | 30.351 | 1.00 | 3.58 | [symbol] |
| ATOM | 3066 | C | CYS | A | 318 | 19.546 | 3.909 | 32.557 | 1.00 | 2.89 | [symbol] |
| ATOM | 3067 | O | CYS | A | 318 | 19.564 | 5.043 | 33.031 | 1.00 | 2.65 | [symbol] |
| ATOM | 3068 | N | ILE | A | 319 | 19.941 | 2.826 | 33.223 | 1.00 | 2.82 | [symbol] |
| ATOM | 3070 | CA | ILE | A | 319 | 20.562 | 2.902 | 34.537 | 1.00 | 3.48 | [symbol] |
| ATOM | 3072 | CB | ILE | A | 319 | 19.903 | 1.905 | 35.512 | 1.00 | 3.96 | [symbol] |
| ATOM | 3074 | CG1 | ILE | A | 319 | 18.398 | 2.176 | 35.628 | 1.00 | 5.82 | [symbol] |
| ATOM | 3077 | CD1 | ILE | A | 319 | 18.023 | 3.175 | 36.621 | 1.00 | 7.54 | [symbol] |
| ATOM | 3081 | CG2 | ILE | A | 319 | 20.612 | 1.932 | 36.846 | 1.00 | 4.63 | [symbol] |
| ATOM | 3085 | C | ILE | A | 319 | 22.012 | 2.515 | 34.311 | 1.00 | 2.77 | [symbol] |
| ATOM | 3086 | O | ILE | A | 319 | 22.282 | 1.418 | 33.815 | 1.00 | 2.85 | [symbol] |
| ATOM | 3087 | N | ARG | A | 320 | 22.930 | 3.417 | 34.653 | 1.00 | 2.38 | [symbol] |
| ATOM | 3089 | CA | ARG | A | 320 | 24.357 | 3.199 | 34.461 | 1.00 | 2.26 | [symbol] |
| ATOM | 3091 | CB | ARG | A | 320 | 24.902 | 4.228 | 33.482 | 1.00 | 2.37 | [symbol] |
| ATOM | 3094 | CG | ARG | A | 320 | 26.360 | 4.064 | 33.145 | 1.00 | 2.44 | [symbol] |
| ATOM | 3097 | CD | ARG | A | 320 | 26.901 | 5.216 | 32.313 | 1.00 | 2.34 | [symbol] |
| ATOM | 3100 | NE | ARG | A | 320 | 26.315 | 5.275 | 30.983 | 1.00 | 2.76 | [symbol] |
| ATOM | 3102 | CZ | ARG | A | 320 | 26.774 | 4.615 | 29.933 | 1.00 | 2.66 | [symbol] |
| ATOM | 3103 | NH1 | ARG | A | 320 | 27.827 | 3.820 | 30.046 | 1.00 | 2.29 | [symbol] |
| ATOM | 3106 | NH2 | ARG | A | 320 | 26.164 | 4.746 | 28.761 | 1.00 | 2.49 | [symbol] |
| ATOM | 3109 | C | ARG | A | 320 | 25.070 | 3.362 | 35.792 | 1.00 | 2.24 | [symbol] |
| ATOM | 3110 | O | ARG | A | 320 | 24.845 | 4.335 | 36.511 | 1.00 | 2.42 | [symbol] |
| ATOM | 3111 | N | THR | A | 321 | 25.943 | 2.419 | 36.115 | 1.00 | 2.12 | [symbol] |
| ATOM | 3113 | CA | THR | A | 321 | 26.667 | 2.490 | 37.390 | 1.00 | 2.25 | [symbol] |
| ATOM | 3115 | CB | THR | A | 321 | 27.462 | 1.218 | 37.665 | 1.00 | 2.06 | [symbol] |
| ATOM | 3117 | OG1 | THR | A | 321 | 28.545 | 1.097 | 36.736 | 1.00 | 2.59 | [symbol] |
| ATOM | 3119 | CG2 | THR | A | 321 | 26.650 | −0.039 | 37.490 | 1.00 | 2.06 | [symbol] |
| ATOM | 3123 | C | THR | A | 321 | 27.605 | 3.696 | 37.421 | 1.00 | 2.65 | [symbol] |
| ATOM | 3124 | O | THR | A | 321 | 28.131 | 4.132 | 36.388 | 1.00 | 2.48 | [symbol] |
| ATOM | 3125 | N | LEU | A | 322 | 27.791 | 4.227 | 38.628 | 1.00 | 3.11 | [symbol] |
| ATOM | 3127 | CA | LEU | A | 322 | 28.667 | 5.360 | 38.890 | 1.00 | 3.87 | [symbol] |
| ATOM | 3129 | CB | LEU | A | 322 | 28.016 | 6.264 | 39.937 | 1.00 | 4.48 | [symbol] |
| ATOM | 3132 | CG | LEU | A | 322 | 27.290 | 7.518 | 39.498 | 1.00 | 6.27 | [symbol] |
| ATOM | 3134 | CD1 | LEU | A | 322 | 26.431 | 7.277 | 38.300 | 1.00 | 9.03 | [symbol] |
| ATOM | 3138 | CD2 | LEU | A | 322 | 26.468 | 7.993 | 40.693 | 1.00 | 6.96 | [symbol] |
| ATOM | 3142 | C | LEU | A | 322 | 29.987 | 4.870 | 39.476 | 1.00 | 4.18 | [symbol] |
| ATOM | 3143 | O | LEU | A | 322 | 30.979 | 5.597 | 39.508 | 1.00 | 4.32 | [symbol] |
| ATOM | 3144 | N | ARG | A | 323 | 29.970 | 3.649 | 39.985 | 1.00 | 4.39 | [symbol] |
| ATOM | 3146 | CA | ARG | A | 323 | 31.132 | 3.070 | 40.646 | 1.00 | 4.63 | [symbol] |
| ATOM | 3148 | CB | ARG | A | 323 | 31.080 | 3.375 | 42.146 | 1.00 | 4.87 | [symbol] |
| ATOM | 3151 | CG | ARG | A | 323 | 29.824 | 2.867 | 42.875 | 1.00 | 6.06 | [symbol] |
| ATOM | 3154 | CD | ARG | A | 323 | 29.726 | 3.355 | 44.329 | 1.00 | 8.55 | [symbol] |
| ATOM | 3157 | NE | ARG | A | 323 | 28.606 | 2.801 | 45.088 | 1.00 | 10.17 | [symbol] |
| ATOM | 3159 | CZ | ARG | A | 323 | 27.802 | 3.516 | 45.885 | 1.00 | 12.22 | [symbol] |
| ATOM | 3160 | NH1 | ARG | A | 323 | 27.966 | 4.828 | 46.029 | 1.00 | 11.79 | [symbol] |
| ATOM | 3163 | NH2 | ARG | A | 323 | 26.815 | 2.918 | 46.539 | 1.00 | 14.22 | [symbol] |
| ATOM | 3166 | C | ARG | A | 323 | 31.090 | 1.571 | 40.424 | 1.00 | 4.50 | [symbol] |
| ATOM | 3167 | O | ARG | A | 323 | 30.161 | 1.064 | 39.814 | 1.00 | 5.16 | [symbol] |
| ATOM | 3168 | N | ALA | A | 324 | 32.084 | 0.846 | 40.915 | 1.00 | 4.31 | [symbol] |
| ATOM | 3170 | CA | ALA | A | 324 | 32.033 | −0.602 | 40.779 | 1.00 | 4.08 | [symbol] |
| ATOM | 3172 | CB | ALA | A | 324 | 33.333 | −1.223 | 41.129 | 1.00 | 3.70 | [symbol] |
| ATOM | 3176 | C | ALA | A | 324 | 30.902 | −1.132 | 41.665 | 1.00 | 4.02 | [symbol] |
| ATOM | 3177 | O | ALA | A | 324 | 30.613 | −0.573 | 42.718 | 1.00 | 3.97 | [symbol] |
| ATOM | 3178 | N | VAL | A | 325 | 30.235 | −2.184 | 41.204 | 1.00 | 4.41 | [symbol] |
| ATOM | 3180 | CA | VAL | A | 325 | 29.097 | −2.754 | 41.907 | 1.00 | 4.52 | [symbol] |
| ATOM | 3182 | CB | VAL | A | 325 | 27.782 | −2.415 | 41.193 | 1.00 | 4.82 | [symbol] |
| ATOM | 3184 | CG1 | VAL | A | 325 | 26.624 | −3.204 | 41.766 | 1.00 | 5.53 | [symbol] |
| ATOM | 3188 | CG2 | VAL | A | 325 | 27.496 | −0.917 | 41.274 | 1.00 | 5.10 | [symbol] |
| ATOM | 3192 | C | VAL | A | 325 | 29.304 | −4.263 | 41.968 | 1.00 | 4.28 | [symbol] |
| ATOM | 3193 | O | VAL | A | 325 | 29.699 | −4.874 | 40.979 | 1.00 | 4.13 | [symbol] |
| ATOM | 3194 | N | GLU | A | 326 | 29.071 | −4.862 | 43.130 | 1.00 | 4.39 | [symbol] |
| ATOM | 3196 | CA | GLU | A | 326 | 29.232 | −6.304 | 43.270 | 1.00 | 4.62 | [symbol] |
| ATOM | 3198 | CB | GLU | A | 326 | 29.656 | −6.670 | 44.685 | 1.00 | 5.39 | [symbol] |
| ATOM | 3201 | CG | GLU | A | 326 | 31.133 | −7.007 | 44.791 | 1.00 | 6.97 | [symbol] |
| ATOM | 3204 | CD | GLU | A | 326 | 31.551 | −8.095 | 43.807 | 1.00 | 9.48 | [symbol] |
| ATOM | 3205 | OE1 | GLU | A | 326 | 30.686 | −8.907 | 43.347 | 1.00 | 8.95 | [symbol] |
| ATOM | 3206 | OE2 | GLU | A | 326 | 32.753 | −8.133 | 43.478 | 1.00 | 11.36 | [symbol] |
| ATOM | 3207 | C | GLU | A | 326 | 27.952 | −7.052 | 42.956 | 1.00 | 4.33 | [symbol] |
| ATOM | 3208 | O | GLU | A | 326 | 26.871 | −6.472 | 42.948 | 1.00 | 4.21 | [symbol] |
| ATOM | 3209 | N | ALA | A | 327 | 28.089 | −8.355 | 42.734 | 1.00 | 3.98 | [symbol] |
| ATOM | 3211 | CA | ALA | A | 327 | 26.940 | −9.226 | 42.518 | 1.00 | 3.84 | [symbol] |
| ATOM | 3213 | CB | ALA | A | 327 | 27.418 | −10.657 | 42.330 | 1.00 | 3.86 | [symbol] |
| ATOM | 3217 | C | ALA | A | 327 | 25.998 | −9.155 | 43.719 | 1.00 | 3.85 | [symbol] |
| ATOM | 3218 | O | ALA | A | 327 | 26.454 | −9.146 | 44.870 | 1.00 | 3.06 | [symbol] |
| ATOM | 3219 | N | ASP | A | 328 | 24.697 | −9.120 | 43.453 | 1.00 | 3.85 | [symbol] |
| ATOM | 3221 | CA | ASP | A | 328 | 23.662 | −9.100 | 44.490 | 1.00 | 4.37 | [symbol] |
| ATOM | 3223 | CB | ASP | A | 328 | 23.813 | −10.313 | 45.399 | 1.00 | 4.71 | [symbol] |
| ATOM | 3226 | CG | ASP | A | 328 | 23.611 | −11.610 | 44.651 | 1.00 | 4.46 | [symbol] |
| ATOM | 3227 | OD1 | ASP | A | 328 | 24.460 | −12.518 | 44.789 | 1.00 | 4.47 | [symbol] |
| ATOM | 3228 | OD2 | ASP | A | 328 | 22.646 | −11.801 | 43.882 | 1.00 | 5.41 | [symbol] |
| ATOM | 3229 | C | ASP | A | 328 | 23.600 | −7.799 | 45.295 | 1.00 | 4.84 | [symbol] |
| ATOM | 3230 | O | ASP | A | 328 | 22.965 | −7.740 | 46.351 | 1.00 | 5.11 | [symbol] |
| ATOM | 3231 | N | GLU | A | 329 | 24.256 | −6.757 | 44.797 | 1.00 | 4.98 | [symbol] |
| ATOM | 3233 | CA | GLU | A | 329 | 24.176 | −5.439 | 45.411 | 1.00 | 4.94 | [symbol] |
| ATOM | 3235 | CB | GLU | A | 329 | 25.431 | −4.620 | 45.086 | 1.00 | 5.24 | [symbol] |
| ATOM | 3238 | CG | GLU | A | 329 | 25.329 | −3.153 | 45.470 | 1.00 | 6.22 | [symbol] |
| ATOM | 3241 | CD | GLU | A | 329 | 26.680 | −2.439 | 45.501 | 1.00 | 7.84 | [symbol] |
| ATOM | 3242 | OE1 | GLU | A | 329 | 27.745 | −3.107 | 45.497 | 1.00 | 6.56 | [symbol] |
| ATOM | 3243 | OE2 | GLU | A | 329 | 26.659 | −1.189 | 45.515 | 1.00 | 9.56 | [symbol] |
| ATOM | 3244 | C | GLU | A | 329 | 22.908 | −4.745 | 44.876 | 1.00 | 4.42 | [symbol] |
| ATOM | 3245 | O | GLU | A | 329 | 22.546 | −4.900 | 43.698 | 1.00 | 4.27 | [symbol] |
| ATOM | 3246 | N | GLU | A | 330 | 22.204 | −4.002 | 45.723 | 1.00 | 3.90 | [symbol] |
| ATOM | 3248 | CA | GLU | A | 330 | 21.009 | −3.293 | 45.234 | 1.00 | 3.66 | [symbol] |
| ATOM | 3250 | CB | GLU | A | 330 | 20.079 | −2.861 | 46.373 | 1.00 | 3.79 | [symbol] |
| ATOM | 3253 | CG | GLU | A | 330 | 18.758 | −2.288 | 45.866 | 1.00 | 3.60 | [symbol] |
| ATOM | 3256 | CD | GLU | A | 330 | 17.722 | −2.054 | 46.950 | 1.00 | 4.19 | [symbol] |
| ATOM | 3257 | OE1 | GLU | A | 330 | 18.039 | −2.252 | 48.125 | 1.00 | 4.83 | [symbol] |
| ATOM | 3258 | OE2 | GLU | A | 330 | 16.567 | −1.680 | 46.626 | 1.00 | 4.44 | [symbol] |
| ATOM | 3259 | C | GLU | A | 330 | 21.439 | −2.076 | 44.415 | 1.00 | 3.53 | [symbol] |
| ATOM | 3260 | O | GLU | A | 330 | 22.347 | −1.328 | 44.822 | 1.00 | 3.55 | [symbol] |
| ATOM | 3261 | N | LEU | A | 331 | 20.796 | −1.891 | 43.264 | 1.00 | 3.45 | [symbol] |
| ATOM | 3263 | CA | LEU | A | 331 | 21.040 | −0.743 | 42.397 | 1.00 | 3.33 | [symbol] |
| ATOM | 3265 | CB | LEU | A | 331 | 20.753 | −1.077 | 40.938 | 1.00 | 3.49 | [symbol] |
| ATOM | 3268 | CG | LEU | A | 331 | 21.633 | −2.168 | 40.332 | 1.00 | 4.45 | [symbol] |
| ATOM | 3270 | CD1 | LEU | A | 331 | 21.415 | −2.275 | 38.821 | 1.00 | 4.93 | [symbol] |
| ATOM | 3274 | CD2 | LEU | A | 331 | 23.105 | −1.933 | 40.668 | 1.00 | 4.93 | [symbol] |
| ATOM | 3278 | C | LEU | A | 331 | 20.149 | 0.400 | 42.838 | 1.00 | 2.89 | [symbol] |
| ATOM | 3279 | O | LEU | A | 331 | 18.930 | 0.239 | 42.968 | 1.00 | 2.74 | [symbol] |
| ATOM | 3280 | N | THR | A | 332 | 20.752 | 1.552 | 43.089 | 1.00 | 2.38 | [symbol] |
| ATOM | 3282 | CA | THR | A | 332 | 19.992 | 2.721 | 43.498 | 1.00 | 2.55 | [symbol] |
| ATOM | 3284 | CB | THR | A | 332 | 20.241 | 3.074 | 44.958 | 1.00 | 2.23 | [symbol] |
| ATOM | 3286 | OG1 | THR | A | 332 | 21.648 | 3.262 | 45.174 | 1.00 | 3.52 | [symbol] |
| ATOM | 3288 | CG2 | THR | A | 332 | 19.851 | 1.934 | 45.860 | 1.00 | 2.12 | [symbol] |
| ATOM | 3292 | C | THR | A | 332 | 20.363 | 3.913 | 42.642 | 1.00 | 2.57 | [symbol] |
| ATOM | 3293 | O | THR | A | 332 | 21.450 | 3.969 | 42.082 | 1.00 | 2.47 | [symbol] |
| ATOM | 3294 | N | VAL | A | 333 | 19.447 | 4.871 | 42.560 | 1.00 | 2.89 | [symbol] |
| ATOM | 3296 | CA | VAL | A | 333 | 19.650 | 6.071 | 41.761 | 1.00 | 3.37 | [symbol] |
| ATOM | 3298 | CB | VAL | A | 333 | 18.935 | 5.964 | 40.381 | 1.00 | 3.63 | [symbol] |
| ATOM | 3300 | CG1 | VAL | A | 333 | 19.195 | 7.192 | 39.540 | 1.00 | 4.18 | [symbol] |
| ATOM | 3304 | CG2 | VAL | A | 333 | 19.375 | 4.712 | 39.631 | 1.00 | 4.44 | [symbol] |
| ATOM | 3308 | C | VAL | A | 333 | 19.063 | 7.272 | 42.494 | 1.00 | 3.05 | [symbol] |
| ATOM | 3309 | O | VAL | A | 333 | 18.110 | 7.138 | 43.250 | 1.00 | 2.77 | [symbol] |
| ATOM | 3310 | N | ALA | A | 334 | 19.626 | 8.448 | 42.258 | 1.00 | 2.99 | [symbol] |
| ATOM | 3312 | CA | ALA | A | 334 | 19.081 | 9.671 | 42.831 | 1.00 | 2.93 | [symbol] |
| ATOM | 3314 | CB | ALA | A | 334 | 20.136 | 10.765 | 42.851 | 1.00 | 3.06 | [symbol] |
| ATOM | 3318 | C | ALA | A | 334 | 17.890 | 10.075 | 41.973 | 1.00 | 2.63 | [symbol] |
| ATOM | 3319 | O | ALA | A | 334 | 18.049 | 10.352 | 40.787 | 1.00 | 3.30 | [symbol] |
| ATOM | 3320 | N | TYR | A | 335 | 16.695 | 10.076 | 42.547 | 1.00 | 2.12 | [symbol] |
| ATOM | 3322 | CA | TYR | A | 335 | 15.488 | 10.421 | 41.772 | 1.00 | 2.13 | [symbol] |
| ATOM | 3324 | CB | TYR | A | 335 | 14.226 | 10.153 | 42.591 | 1.00 | 2.12 | [symbol] |
| ATOM | 3327 | CG | TYR | A | 335 | 13.822 | 8.679 | 42.649 | 1.00 | 2.19 | [symbol] |
| ATOM | 3328 | CD1 | TYR | A | 335 | 14.659 | 7.669 | 42.172 | 1.00 | 2.05 | [symbol] |
| ATOM | 3330 | CE1 | TYR | A | 335 | 14.288 | 6.333 | 42.253 | 1.00 | 2.13 | [symbol] |
| ATOM | 3332 | CZ | TYR | A | 335 | 13.062 | 6.001 | 42.802 | 1.00 | 2.11 | [symbol] |
| ATOM | 3333 | OH | TYR | A | 335 | 12.679 | 4.679 | 42.880 | 1.00 | 2.37 | [symbol] |
| ATOM | 3335 | CE2 | TYR | A | 335 | 12.227 | 6.983 | 43.280 | 1.00 | 2.00 | [symbol] |
| ATOM | 3337 | CD2 | TYR | A | 335 | 12.605 | 8.302 | 43.201 | 1.00 | 2.00 | [symbol] |
| ATOM | 3339 | C | TYR | A | 335 | 15.537 | 11.863 | 41.250 | 1.00 | 2.06 | [symbol] |
| ATOM | 3340 | O | TYR | A | 335 | 14.941 | 12.187 | 40.210 | 1.00 | 2.37 | [symbol] |
| ATOM | 3341 | N | GLY | A | 336 | 16.249 | 12.730 | 41.965 | 1.00 | 2.07 | [symbol] |
| ATOM | 3343 | CA | GLY | A | 336 | 16.435 | 14.105 | 41.538 | 1.00 | 2.14 | [symbol] |
| ATOM | 3346 | C | GLY | A | 336 | 15.197 | 14.964 | 41.409 | 1.00 | 2.34 | [symbol] |
| ATOM | 3347 | O | GLY | A | 336 | 15.019 | 15.632 | 40.396 | 1.00 | 2.51 | [symbol] |
| ATOM | 3348 | N | TYR | A | 337 | 14.344 | 14.964 | 42.425 | 1.00 | 2.25 | [symbol] |
| ATOM | 3350 | CA | TYR | A | 337 | 13.200 | 15.861 | 42.424 | 1.00 | 2.45 | [symbol] |
| ATOM | 3352 | CB | TYR | A | 337 | 12.150 | 15.412 | 43.440 | 1.00 | 2.38 | [symbol] |
| ATOM | 3355 | CG | TYR | A | 337 | 11.528 | 14.084 | 43.097 | 1.00 | 2.24 | [symbol] |
| ATOM | 3356 | CD1 | TYR | A | 337 | 11.490 | 13.046 | 44.013 | 1.00 | 2.21 | [symbol] |
| ATOM | 3358 | CE1 | TYR | A | 337 | 10.916 | 11.828 | 43.694 | 1.00 | 2.44 | [symbol] |
| ATOM | 3360 | CZ | TYR | A | 337 | 10.367 | 11.648 | 42.449 | 1.00 | 2.60 | [symbol] |
| ATOM | 3361 | OH | TYR | A | 337 | 9.790 | 10.446 | 42.091 | 1.00 | 2.91 | [symbol] |
| ATOM | 3363 | CE2 | TYR | A | 337 | 10.396 | 12.668 | 41.530 | 1.00 | 2.55 | [symbol] |
| ATOM | 3365 | CD2 | TYR | A | 337 | 10.963 | 13.867 | 41.847 | 1.00 | 2.67 | [symbol] |
| ATOM | 3367 | C | TYR | A | 337 | 13.721 | 17.255 | 42.754 | 1.00 | 2.50 | [symbol] |
| ATOM | 3368 | O | TYR | A | 337 | 14.841 | 17.394 | 43.269 | 1.00 | 2.54 | [symbol] |
| ATOM | 3369 | N | ASP | A | 338 | 12.922 | 18.275 | 42.463 | 1.00 | 2.61 | [symbol] |
| ATOM | 3371 | CA | ASP | A | 338 | 13.279 | 19.658 | 42.726 | 1.00 | 3.24 | [symbol] |
| ATOM | 3373 | CB | ASP | A | 338 | 12.413 | 20.600 | 41.906 | 1.00 | 3.78 | [symbol] |
| ATOM | 3376 | CG | ASP | A | 338 | 12.916 | 22.029 | 41.916 | 1.00 | 4.73 | [symbol] |
| ATOM | 3377 | OD1 | ASP | A | 338 | 13.709 | 22.399 | 42.804 | 1.00 | 4.71 | [symbol] |
| ATOM | 3378 | OD2 | ASP | A | 338 | 12.539 | 22.873 | 41.070 | 1.00 | 6.96 | [symbol] |
| ATOM | 3379 | C | ASP | A | 338 | 13.128 | 19.964 | 44.214 | 1.00 | 3.59 | [symbol] |
| ATOM | 3380 | O | ASP | A | 338 | 12.020 | 19.993 | 44.750 | 1.00 | 3.63 | [symbol] |
| ATOM | 3381 | N | HIS | A | 339 | 14.262 | 20.207 | 44.865 | 1.00 | 3.16 | [symbol] |
| ATOM | 3383 | CA | HIS | A | 339 | 14.304 | 20.488 | 46.296 | 1.00 | 3.75 | [symbol] |
| ATOM | 3385 | CB | HIS | A | 339 | 15.695 | 20.166 | 46.843 | 1.00 | 3.35 | [symbol] |
| ATOM | 3388 | CG | HIS | A | 339 | 16.044 | 18.711 | 46.790 | 1.00 | 3.17 | [symbol] |
| ATOM | 3389 | ND1 | HIS | A | 339 | 17.254 | 18.223 | 47.230 | 1.00 | 2.63 | [symbol] |
| ATOM | 3391 | CE1 | HIS | A | 339 | 17.288 | 16.915 | 47.057 | 1.00 | 2.70 | [symbol] |
| ATOM | 3393 | NE2 | HIS | A | 339 | 16.143 | 16.534 | 46.522 | 1.00 | 2.61 | [symbol] |
| ATOM | 3395 | CD2 | HIS | A | 339 | 15.350 | 17.639 | 46.338 | 1.00 | 2.29 | [symbol] |
| ATOM | 3397 | C | HIS | A | 339 | 13.972 | 21.931 | 46.640 | 1.00 | 4.29 | [symbol] |
| ATOM | 3398 | O | HIS | A | 339 | 13.708 | 22.237 | 47.799 | 1.00 | 4.47 | [symbol] |
| ATOM | 3399 | N | SER | A | 340 | 13.989 | 22.813 | 45.642 | 1.00 | 4.86 | [symbol] |
| ATOM | 3401 | CA | SER | A | 340 | 13.725 | 24.228 | 45.875 | 1.00 | 5.67 | [symbol] |
| ATOM | 3403 | CB | SER | A | 340 | 15.034 | 24.966 | 46.119 | 1.00 | 5.47 | [symbol] |
| ATOM | 3406 | OG | SER | A | 340 | 14.765 | 26.299 | 46.505 | 1.00 | 5.21 | [symbol] |
| ATOM | 3408 | C | SER | A | 340 | 12.992 | 24.922 | 44.726 | 1.00 | 6.73 | [symbol] |
| ATOM | 3409 | O | SER | A | 340 | 13.558 | 25.757 | 44.031 | 1.00 | 6.15 | [symbol] |
| ATOM | 3410 | N | PRO | A | 341 | 11.735 | 24.570 | 44.514 | 1.00 | 8.49 | [symbol] |
| ATOM | 3411 | CA | PRO | A | 341 | 10.935 | 25.205 | 43.464 | 1.00 | 9.84 | [symbol] |
| ATOM | 3413 | CB | PRO | A | 341 | 9.548 | 24.564 | 43.621 | 1.00 | 9.67 | [symbol] |
| ATOM | 3416 | CG | PRO | A | 341 | 9.663 | 23.509 | 44.656 | 1.00 | 9.19 | [symbol] |
| ATOM | 3419 | CD | PRO | A | 341 | 11.008 | 23.538 | 45.251 | 1.00 | 8.47 | [symbol] |
| ATOM | 3422 | C | PRO | A | 341 | 10.906 | 26.729 | 43.636 | 1.00 | 11.40 | [symbol] |
| ATOM | 3423 | O | PRO | A | 341 | 11.054 | 27.212 | 44.752 | 1.00 | 11.46 | [symbol] |
| ATOM | 3424 | N | PRO | A | 342 | 10.702 | 27.460 | 42.539 | 1.00 | 13.36 | [symbol] |
| ATOM | 3425 | CA | PRO | A | 342 | 10.795 | 28.924 | 42.514 | 1.00 | 14.64 | [symbol] |
| ATOM | 3427 | CB | PRO | A | 342 | 10.413 | 29.298 | 41.083 | 1.00 | 14.54 | [symbol] |
| ATOM | 3430 | CG | PRO | A | 342 | 10.091 | 28.070 | 40.386 | 1.00 | 14.08 | [symbol] |
| ATOM | 3433 | CD | PRO | A | 342 | 10.255 | 26.912 | 41.264 | 1.00 | 13.41 | [symbol] |
| ATOM | 3436 | C | PRO | A | 342 | 9.692 | 29.343 | 43.362 | 1.00 | 15.96 | [symbol] |
| ATOM | 3437 | O | PRO | A | 342 | 9.633 | 30.393 | 44.022 | 1.00 | 16.30 | [symbol] |
| ATOM | 3438 | N | GLY | A | 343 | 8.735 | 28.442 | 43.240 | 1.00 | 17.38 | [symbol] |
| ATOM | 3440 | CA | GLY | A | 343 | 7.569 | 28.478 | 44.029 | 1.00 | 18.23 | [symbol] |
| ATOM | 3443 | C | GLY | A | 343 | 8.228 | 28.995 | 45.268 | 1.00 | 18.94 | [symbol] |
| ATOM | 3444 | O | GLY | A | 343 | 9.434 | 29.246 | 45.343 | 1.00 | 19.46 | [symbol] |
| ATOM | 3445 | N | LYS | A | 344 | 7.428 | 29.144 | 46.274 | 1.00 | 19.60 | [symbol] |
| ATOM | 3447 | CA | LYS | A | 344 | 7.914 | 29.735 | 47.463 | 1.00 | 20.09 | [symbol] |
| ATOM | 3449 | CB | LYS | A | 344 | 6.668 | 30.125 | 48.244 | 1.00 | 20.41 | [symbol] |
| ATOM | 3452 | CG | LYS | A | 344 | 6.315 | 29.099 | 49.302 | 1.00 | 21.21 | [symbol] |
| ATOM | 3455 | CD | LYS | A | 344 | 5.275 | 29.602 | 50.247 | 1.00 | 22.19 | [symbol] |
| ATOM | 3458 | CE | LYS | A | 344 | 4.668 | 28.474 | 51.058 | 1.00 | 22.78 | [symbol] |
| ATOM | 3461 | NZ | LYS | A | 344 | 3.561 | 28.978 | 51.917 | 1.00 | 22.97 | [symbol] |
| ATOM | 3465 | C | LYS | A | 344 | 8.632 | 28.654 | 48.255 | 1.00 | 20.09 | [symbol] |
| ATOM | 3466 | O | LYS | A | 344 | 9.787 | 28.762 | 48.682 | 1.00 | 20.34 | [symbol] |
| ATOM | 3467 | N | SER | A | 345 | 7.861 | 27.577 | 48.261 | 1.00 | 19.82 | [symbol] |
| ATOM | 3469 | CA | SER | A | 345 | 7.728 | 26.467 | 49.184 | 1.00 | 19.48 | [symbol] |
| ATOM | 3471 | CB | SER | A | 345 | 6.371 | 26.002 | 48.682 | 1.00 | 19.59 | [symbol] |
| ATOM | 3474 | OG | SER | A | 345 | 6.417 | 26.059 | 47.251 | 1.00 | 20.84 | [symbol] |
| ATOM | 3476 | C | SER | A | 345 | 8.360 | 25.117 | 49.402 | 1.00 | 18.72 | [symbol] |
| ATOM | 3477 | O | SER | A | 345 | 7.650 | 24.234 | 49.879 | 1.00 | 19.17 | [symbol] |
| ATOM | 3478 | N | GLY | A | 346 | 9.612 | 24.864 | 49.135 | 1.00 | 17.45 | [symbol] |
| ATOM | 3480 | CA | GLY | A | 346 | 10.055 | 23.530 | 49.496 | 1.00 | 16.41 | [symbol] |
| ATOM | 3483 | C | GLY | A | 346 | 9.892 | 22.517 | 48.382 | 1.00 | 15.40 | [symbol] |
| ATOM | 3484 | O | GLY | A | 346 | 9.197 | 22.747 | 47.397 | 1.00 | 15.23 | [symbol] |
| ATOM | 3485 | N | PRO | A | 347 | 10.529 | 21.369 | 48.560 | 1.00 | 14.06 | [symbol] |
| ATOM | 3486 | CA | PRO | A | 347 | 10.630 | 20.358 | 47.508 | 1.00 | 13.20 | [symbol] |
| ATOM | 3488 | CB | PRO | A | 347 | 11.447 | 19.236 | 48.160 | 1.00 | 13.19 | [symbol] |
| ATOM | 3491 | CG | PRO | A | 347 | 11.825 | 19.687 | 49.517 | 1.00 | 13.71 | [symbol] |
| ATOM | 3494 | CD | PRO | A | 347 | 11.199 | 20.970 | 49.801 | 1.00 | 14.23 | [symbol] |
| ATOM | 3497 | C | PRO | A | 347 | 9.322 | 19.797 | 46.988 | 1.00 | 12.24 | [symbol] |
| ATOM | 3498 | O | PRO | A | 347 | 8.428 | 19.461 | 47.754 | 1.00 | 12.46 | [symbol] |
| ATOM | 3499 | N | GLU | A | 348 | 9.227 | 19.698 | 45.669 | 1.00 | 11.28 | [symbol] |
| ATOM | 3501 | CA | GLU | A | 348 | 8.063 | 19.073 | 45.047 | 1.00 | 10.48 | [symbol] |
| ATOM | 3503 | CB | GLU | A | 348 | 7.728 | 19.695 | 43.700 | 1.00 | 11.42 | [symbol] |
| ATOM | 3506 | CG | GLU | A | 348 | 6.239 | 20.027 | 43.515 | 1.00 | 14.16 | [symbol] |
| ATOM | 3509 | CD | GLU | A | 348 | 5.301 | 19.258 | 44.447 | 1.00 | 17.81 | [symbol] |
| ATOM | 3510 | OE1 | GLU | A | 348 | 5.099 | 18.042 | 44.248 | 1.00 | 20.08 | [symbol] |
| ATOM | 3511 | OE2 | GLU | A | 348 | 4.749 | 19.879 | 45.386 | 1.00 | 19.94 | [symbol] |
| ATOM | 3512 | C | GLU | A | 348 | 8.448 | 17.609 | 44.902 | 1.00 | 8.57 | [symbol] |
| ATOM | 3513 | O | GLU | A | 348 | 9.138 | 17.214 | 43.968 | 1.00 | 7.67 | [symbol] |
| ATOM | 3514 | N | ALA | A | 349 | 8.041 | 16.810 | 45.874 | 1.00 | 7.53 | [symbol] |
| ATOM | 3516 | CA | ALA | A | 349 | 8.433 | 15.419 | 45.919 | 1.00 | 6.82 | [symbol] |
| ATOM | 3518 | CB | ALA | A | 349 | 9.829 | 15.300 | 46.541 | 1.00 | 6.96 | [symbol] |
| ATOM | 3522 | C | ALA | A | 349 | 7.430 | 14.658 | 46.753 | 1.00 | 6.14 | [symbol] |
| ATOM | 3523 | O | ALA | A | 349 | 6.677 | 15.252 | 47.524 | 1.00 | 5.39 | [symbol] |
| ATOM | 3524 | N | PRO | A | 350 | 7.427 | 13.339 | 46.618 | 1.00 | 6.01 | [symbol] |
| ATOM | 3525 | CA | PRO | A | 350 | 6.539 | 12.502 | 47.408 | 1.00 | 5.75 | [symbol] |
| ATOM | 3527 | CB | PRO | A | 350 | 6.814 | 11.089 | 46.884 | 1.00 | 6.19 | [symbol] |
| ATOM | 3530 | CG | PRO | A | 350 | 7.616 | 11.243 | 45.665 | 1.00 | 6.43 | [symbol] |
| ATOM | 3533 | CD | PRO | A | 350 | 8.290 | 12.542 | 45.737 | 1.00 | 6.39 | [symbol] |
| ATOM | 3536 | C | PRO | A | 350 | 6.958 | 12.613 | 48.865 | 1.00 | 5.09 | [symbol] |
| ATOM | 3537 | O | PRO | A | 350 | 8.127 | 12.904 | 49.136 | 1.00 | 4.77 | [symbol] |
| ATOM | 3538 | N | GLU | A | 351 | 6.025 | 12.392 | 49.777 | 1.00 | 4.61 | [symbol] |
| ATOM | 3540 | CA | GLU | A | 351 | 6.289 | 12.547 | 51.198 | 1.00 | 4.26 | [symbol] |
| ATOM | 3542 | CB | GLU | A | 351 | 5.007 | 12.350 | 52.004 | 1.00 | 4.18 | [symbol] |
| ATOM | 3545 | CG | GLU | A | 351 | 5.192 | 12.539 | 53.512 | 1.00 | 3.77 | [symbol] |
| ATOM | 3548 | CD | GLU | A | 351 | 5.700 | 13.918 | 53.922 | 1.00 | 3.90 | [symbol] |
| ATOM | 3549 | OE1 | GLU | A | 351 | 5.729 | 14.847 | 53.088 | 1.00 | 3.41 | [symbol] |
| ATOM | 3550 | OE2 | GLU | A | 351 | 6.079 | 14.078 | 55.111 | 1.00 | 4.18 | [symbol] |
| ATOM | 3551 | C | GLU | A | 351 | 7.399 | 11.637 | 51.723 | 1.00 | 3.94 | [symbol] |
| ATOM | 3552 | O | GLU | A | 351 | 8.212 | 12.067 | 52.542 | 1.00 | 3.91 | [symbol] |
| ATOM | 3553 | N | TRP | A | 352 | 7.439 | 10.386 | 51.268 | 1.00 | 3.48 | [symbol] |
| ATOM | 3555 | CA | TRP | A | 352 | 8.464 | 9.448 | 51.727 | 1.00 | 3.30 | [symbol] |
| ATOM | 3557 | CB | TRP | A | 352 | 8.244 | 8.051 | 51.132 | 1.00 | 3.23 | [symbol] |
| ATOM | 3560 | CG | TRP | A | 352 | 8.373 | 7.984 | 49.655 | 1.00 | 2.44 | [symbol] |
| ATOM | 3561 | CD1 | TRP | A | 352 | 7.354 | 7.966 | 48.734 | 1.00 | 2.73 | [symbol] |
| ATOM | 3563 | NE1 | TRP | A | 352 | 7.872 | 7.904 | 47.464 | 1.00 | 2.26 | [symbol] |
| ATOM | 3565 | CE2 | TRP | A | 352 | 9.242 | 7.885 | 47.545 | 1.00 | 2.19 | [symbol] |
| ATOM | 3566 | CD2 | TRP | A | 352 | 9.587 | 7.923 | 48.907 | 1.00 | 2.67 | [symbol] |
| ATOM | 3567 | CE3 | TRP | A | 352 | 10.950 | 7.919 | 49.251 | 1.00 | 2.00 | [symbol] |
| ATOM | 3569 | CZ3 | TRP | A | 352 | 11.888 | 7.860 | 48.247 | 1.00 | 2.01 | [symbol] |
| ATOM | 3571 | CH2 | TRP | A | 352 | 11.510 | 7.818 | 46.900 | 1.00 | 2.08 | [symbol] |
| ATOM | 3573 | CZ2 | TRP | A | 352 | 10.193 | 7.823 | 46.534 | 1.00 | 2.44 | [symbol] |
| ATOM | 3575 | C | TRP | A | 352 | 9.872 | 9.978 | 51.407 | 1.00 | 3.05 | [symbol] |
| ATOM | 3576 | O | TRP | A | 352 | 10.821 | 9.749 | 52.161 | 1.00 | 2.69 | [symbol] |
| ATOM | 3577 | N | TYR | A | 353 | 9.984 | 10.686 | 50.286 | 1.00 | 2.79 | [symbol] |
| ATOM | 3579 | CA | TYR | A | 353 | 11.239 | 11.243 | 49.798 | 1.00 | 2.97 | [symbol] |
| ATOM | 3581 | CB | TYR | A | 353 | 11.074 | 11.636 | 48.332 | 1.00 | 2.84 | [symbol] |
| ATOM | 3584 | CG | TYR | A | 353 | 12.258 | 12.302 | 47.667 | 1.00 | 2.38 | [symbol] |
| ATOM | 3585 | CD1 | TYR | A | 353 | 13.044 | 11.602 | 46.758 | 1.00 | 2.40 | [symbol] |
| ATOM | 3587 | CE1 | TYR | A | 353 | 14.092 | 12.210 | 46.110 | 1.00 | 2.38 | [symbol] |
| ATOM | 3589 | CZ | TYR | A | 353 | 14.346 | 13.539 | 46.339 | 1.00 | 2.34 | [symbol] |
| ATOM | 3590 | OH | TYR | A | 353 | 15.372 | 14.143 | 45.669 | 1.00 | 3.35 | [symbol] |
| ATOM | 3592 | CE2 | TYR | A | 353 | 13.561 | 14.265 | 47.205 | 1.00 | 2.14 | [symbol] |
| ATOM | 3594 | CD2 | TYR | A | 353 | 12.520 | 13.646 | 47.858 | 1.00 | 2.08 | [symbol] |
| ATOM | 3596 | C | TYR | A | 353 | 11.620 | 12.449 | 50.640 | 1.00 | 3.20 | [symbol] |
| ATOM | 3597 | O | TYR | A | 353 | 12.780 | 12.621 | 50.995 | 1.00 | 3.31 | [symbol] |
| ATOM | 3598 | N | GLN | A | 354 | 10.632 | 13.274 | 50.965 | 1.00 | 3.72 | [symbol] |
| ATOM | 3600 | CA | GLN | A | 354 | 10.862 | 14.427 | 51.829 | 1.00 | 4.18 | [symbol] |
| ATOM | 3602 | CB | GLN | A | 354 | 9.589 | 15.248 | 51.985 | 1.00 | 4.39 | [symbol] |
| ATOM | 3605 | CG | GLN | A | 354 | 9.064 | 15.734 | 50.649 | 1.00 | 5.35 | [symbol] |
| ATOM | 3608 | CD | GLN | A | 354 | 8.216 | 16.977 | 50.735 | 1.00 | 6.61 | [symbol] |
| ATOM | 3609 | OE1 | GLN | A | 354 | 8.524 | 17.899 | 51.485 | 1.00 | 8.35 | [symbol] |
| ATOM | 3610 | NE2 | GLN | A | 354 | 7.164 | 17.022 | 49.933 | 1.00 | 6.18 | [symbol] |
| ATOM | 3613 | C | GLN | A | 354 | 11.365 | 13.965 | 53.195 | 1.00 | 4.32 | [symbol] |
| ATOM | 3614 | O | GLN | A | 354 | 12.252 | 14.591 | 53.771 | 1.00 | 4.28 | [symbol] |
| ATOM | 3615 | N | VAL | A | 355 | 10.795 | 12.871 | 53.706 | 1.00 | 4.44 | [symbol] |
| ATOM | 3617 | CA | VAL | A | 355 | 11.211 | 12.316 | 55.001 | 1.00 | 4.83 | [symbol] |
| ATOM | 3619 | CB | VAL | A | 355 | 10.322 | 11.120 | 55.416 | 1.00 | 4.90 | [symbol] |
| ATOM | 3621 | CG1 | VAL | A | 355 | 10.953 | 10.366 | 56.603 | 1.00 | 5.07 | [symbol] |
| ATOM | 3625 | CG2 | VAL | A | 355 | 8.928 | 11.585 | 55.764 | 1.00 | 4.57 | [symbol] |
| ATOM | 3629 | C | VAL | A | 355 | 12.666 | 11.842 | 54.947 | 1.00 | 4.96 | [symbol] |
| ATOM | 3630 | O | VAL | A | 355 | 13.457 | 12.124 | 55.853 | 1.00 | 4.94 | [symbol] |
| ATOM | 3631 | N | GLU | A | 356 | 13.005 | 11.105 | 53.887 | 1.00 | 5.36 | [symbol] |
| ATOM | 3633 | CA | GLU | A | 356 | 14.367 | 10.585 | 53.709 | 1.00 | 5.54 | [symbol] |
| ATOM | 3635 | CB | GLU | A | 356 | 14.446 | 9.650 | 52.495 | 1.00 | 6.54 | [symbol] |
| ATOM | 3638 | CG | GLU | A | 356 | 15.780 | 8.917 | 52.429 | 1.00 | 9.22 | [symbol] |
| ATOM | 3641 | CD | GLU | A | 356 | 15.801 | 7.726 | 51.489 | 1.00 | 13.26 | [symbol] |
| ATOM | 3642 | OE1 | GLU | A | 356 | 16.176 | 7.917 | 50.315 | 1.00 | 15.69 | [symbol] |
| ATOM | 3643 | OE2 | GLU | A | 356 | 15.493 | 6.590 | 51.933 | 1.00 | 15.43 | [symbol] |
| ATOM | 3644 | C | GLU | A | 356 | 15.349 | 11.737 | 53.541 | 1.00 | 4.88 | [symbol] |
| ATOM | 3645 | O | GLU | A | 356 | 16.484 | 11.695 | 54.048 | 1.00 | 4.16 | [symbol] |
| ATOM | 3646 | N | LEU | A | 357 | 14.913 | 12.784 | 52.851 | 1.00 | 4.21 | [symbol] |
| ATOM | 3648 | CA | LEU | A | 357 | 15.751 | 13.967 | 52.671 | 1.00 | 4.57 | [symbol] |
| ATOM | 3650 | CB | LEU | A | 357 | 15.024 | 15.006 | 51.825 | 1.00 | 3.91 | [symbol] |
| ATOM | 3653 | CG | LEU | A | 357 | 15.813 | 16.279 | 51.499 | 1.00 | 3.60 | [symbol] |
| ATOM | 3655 | CD1 | LEU | A | 357 | 17.210 | 15.951 | 51.005 | 1.00 | 3.39 | [symbol] |
| ATOM | 3659 | CD2 | LEU | A | 357 | 15.093 | 17.144 | 50.480 | 1.00 | 4.35 | [symbol] |
| ATOM | 3663 | C | LEU | A | 357 | 16.112 | 14.579 | 54.032 | 1.00 | 4.95 | [symbol] |
| ATOM | 3664 | O | LEU | A | 357 | 17.270 | 14.937 | 54.294 | 1.00 | 5.53 | [symbol] |
| ATOM | 3665 | N | LYS | A | 358 | 15.108 | 14.694 | 54.897 | 1.00 | 5.80 | [symbol] |
| ATOM | 3667 | CA | LYS | A | 358 | 15.295 | 15.243 | 56.242 | 1.00 | 6.34 | [symbol] |
| ATOM | 3669 | CB | LYS | A | 358 | 13.954 | 15.311 | 56.986 | 1.00 | 6.79 | [symbol] |
| ATOM | 3672 | CG | LYS | A | 358 | 14.039 | 15.852 | 58.408 | 1.00 | 8.85 | [symbol] |
| ATOM | 3675 | CD | LYS | A | 358 | 12.667 | 15.963 | 59.081 | 1.00 | 11.56 | [symbol] |
| ATOM | 3678 | CE | LYS | A | 358 | 12.778 | 15.922 | 60.608 | 1.00 | 13.45 | [symbol] |
| ATOM | 3681 | NZ | LYS | A | 358 | 11.446 | 16.046 | 61.278 | 1.00 | 15.20 | [symbol] |
| ATOM | 3685 | C | LYS | A | 358 | 16.278 | 14.381 | 57.021 | 1.00 | 6.23 | [symbol] |
| ATOM | 3686 | O | LYS | A | 358 | 17.210 | 14.897 | 57.647 | 1.00 | 6.46 | [symbol] |
| ATOM | 3687 | N | ALA | A | 359 | 16.055 | 13.069 | 56.982 | 1.00 | 6.07 | [symbol] |
| ATOM | 3689 | CA | ALA | A | 359 | 16.879 | 12.101 | 57.687 | 1.00 | 6.00 | [symbol] |
| ATOM | 3691 | CB | ALA | A | 359 | 16.277 | 10.724 | 57.587 | 1.00 | 6.19 | [symbol] |
| ATOM | 3695 | C | ALA | A | 359 | 18.303 | 12.091 | 57.143 | 1.00 | 6.09 | [symbol] |
| ATOM | 3696 | O | ALA | A | 359 | 19.265 | 12.036 | 57.907 | 1.00 | 5.68 | [symbol] |
| ATOM | 3697 | N | PHE | A | 360 | 18.443 | 12.145 | 55.824 | 1.00 | 5.93 | [symbol] |
| ATOM | 3699 | CA | PHE | A | 360 | 19.774 | 12.143 | 55.219 | 1.00 | 6.25 | [symbol] |
| ATOM | 3701 | CB | PHE | A | 360 | 19.692 | 12.075 | 53.691 | 1.00 | 6.09 | [symbol] |
| ATOM | 3704 | CG | PHE | A | 360 | 21.035 | 12.164 | 53.015 | 1.00 | 5.08 | [symbol] |
| ATOM | 3705 | CD1 | PHE | A | 360 | 21.974 | 11.152 | 53.164 | 1.00 | 5.53 | [symbol] |
| ATOM | 3707 | CE1 | PHE | A | 360 | 23.222 | 11.241 | 52.557 | 1.00 | 5.05 | [symbol] |
| ATOM | 3709 | CZ | PHE | A | 360 | 23.541 | 12.349 | 51.800 | 1.00 | 5.36 | [symbol] |
| ATOM | 3711 | CE2 | PHE | A | 360 | 22.616 | 13.362 | 51.643 | 1.00 | 5.78 | [symbol] |
| ATOM | 3713 | CD2 | PHE | A | 360 | 21.371 | 13.270 | 52.258 | 1.00 | 5.37 | [symbol] |
| ATOM | 3715 | C | PHE | A | 360 | 20.549 | 13.395 | 55.642 | 1.00 | 6.88 | [symbol] |
| ATOM | 3716 | O | PHE | A | 360 | 21.688 | 13.311 | 56.106 | 1.00 | 7.37 | [symbol] |
| ATOM | 3717 | N | GLN | A | 361 | 19.913 | 14.553 | 55.504 | 1.00 | 7.57 | [symbol] |
| ATOM | 3719 | CA | GLN | A | 361 | 20.535 | 15.826 | 55.850 | 1.00 | 8.38 | [symbol] |
| ATOM | 3721 | CB | GLN | A | 361 | 19.603 | 16.971 | 55.489 | 1.00 | 7.94 | [symbol] |
| ATOM | 3724 | CG | GLN | A | 361 | 19.627 | 17.293 | 53.983 | 1.00 | 7.07 | [symbol] |
| ATOM | 3727 | CD | GLN | A | 361 | 21.036 | 17.618 | 53.478 | 1.00 | 5.92 | [symbol] |
| ATOM | 3728 | OE1 | GLN | A | 361 | 21.496 | 17.078 | 52.467 | 1.00 | 7.96 | [symbol] |
| ATOM | 3729 | NE2 | GLN | A | 361 | 21.702 | 18.503 | 54.166 | 1.00 | 3.18 | [symbol] |
| ATOM | 3732 | C | GLN | A | 361 | 20.932 | 15.897 | 57.325 | 1.00 | 9.69 | [symbol] |
| ATOM | 3733 | O | GLN | A | 361 | 21.821 | 16.664 | 57.692 | 1.00 | 9.62 | [symbol] |
| ATOM | 3734 | N | ALA | A | 362 | 20.273 | 15.099 | 58.159 | 1.00 | 11.55 | [symbol] |
| ATOM | 3736 | CA | ALA | A | 362 | 20.615 | 15.019 | 59.590 | 1.00 | 13.25 | [symbol] |
| ATOM | 3738 | CB | ALA | A | 362 | 19.511 | 14.306 | 60.361 | 1.00 | 13.47 | [symbol] |
| ATOM | 3742 | C | ALA | A | 362 | 21.944 | 14.302 | 59.788 | 1.00 | 14.65 | [symbol] |
| ATOM | 3743 | O | ALA | A | 362 | 22.567 | 14.414 | 60.852 | 1.00 | 15.14 | [symbol] |
| ATOM | 3744 | N | THR | A | 363 | 22.382 | 13.561 | 58.772 | 1.00 | 16.29 | [symbol] |
| ATOM | 3746 | CA | THR | A | 363 | 23.667 | 12.859 | 58.813 | 1.00 | 17.77 | [symbol] |
| ATOM | 3748 | CB | THR | A | 363 | 23.600 | 11.548 | 58.031 | 1.00 | 17.95 | [symbol] |
| ATOM | 3750 | OG1 | THR | A | 363 | 22.493 | 10.765 | 58.497 | 1.00 | 18.76 | [symbol] |
| ATOM | 3752 | CG2 | THR | A | 363 | 24.814 | 10.663 | 58.314 | 1.00 | 18.15 | [symbol] |
| ATOM | 3756 | C | THR | A | 363 | 24.732 | 13.750 | 58.195 | 1.00 | 18.76 | [symbol] |
| ATOM | 3757 | O | THR | A | 363 | 25.922 | 13.421 | 58.208 | 1.00 | 18.78 | [symbol] |
| ATOM | 3758 | N | GLN | A | 364 | 24.283 | 14.871 | 57.640 | 1.00 | 19.77 | [symbol] |
| ATOM | 3760 | CA | GLN | A | 364 | 25.158 | 15.851 | 57.022 | 1.00 | 20.96 | [symbol] |
| ATOM | 3762 | CB | GLN | A | 364 | 24.616 | 16.229 | 55.640 | 1.00 | 21.24 | [symbol] |
| ATOM | 3765 | CG | GLN | A | 364 | 25.490 | 15.801 | 54.446 | 1.00 | 22.43 | [symbol] |
| ATOM | 3768 | CD | GLN | A | 364 | 25.668 | 14.294 | 54.277 | 1.00 | 23.32 | [symbol] |
| ATOM | 3769 | OE1 | GLN | A | 364 | 25.472 | 13.516 | 55.210 | 1.00 | 23.46 | [symbol] |
| ATOM | 3770 | NE2 | GLN | A | 364 | 26.060 | 13.889 | 53.076 | 1.00 | 24.13 | [symbol] |
| ATOM | 3773 | C | GLN | A | 364 | 25.312 | 17.110 | 57.902 | 1.00 | 21.55 | [symbol] |
| ATOM | 3774 | O | GLN | A | 364 | 26.401 | 17.645 | 58.011 | 1.00 | 21.55 | [symbol] |
| ATOM | 3775 | N | GLN | A | 365 | 24.219 | 17.542 | 58.539 | 1.00 | 22.62 | [symbol] |
| ATOM | 3777 | CA | GLN | A | 365 | 24.114 | 18.807 | 59.294 | 1.00 | 23.44 | [symbol] |
| ATOM | 3779 | CB | GLN | A | 365 | 25.287 | 19.137 | 60.218 | 1.00 | 23.70 | [symbol] |
| ATOM | 3782 | CG | GLN | A | 365 | 24.879 | 19.207 | 61.721 | 1.00 | 24.10 | [symbol] |
| ATOM | 3785 | CD | GLN | A | 365 | 24.542 | 20.613 | 62.236 | 1.00 | 24.69 | [symbol] |
| ATOM | 3786 | OE1 | GLN | A | 365 | 23.620 | 21.268 | 61.738 | 1.00 | 25.16 | [symbol] |
| ATOM | 3787 | NE2 | GLN | A | 365 | 25.279 | 21.061 | 63.257 | 1.00 | 24.67 | [symbol] |
| ATOM | 3790 | C | GLN | A | 365 | 23.877 | 19.909 | 58.281 | 1.00 | 24.03 | [symbol] |
| ATOM | 3791 | O | GLN | A | 365 | 24.709 | 20.190 | 57.421 | 1.00 | 24.63 | [symbol] |
| ATOM | 3792 | N | LYS | A | 366 | 22.717 | 20.524 | 58.396 | 1.00 | 24.21 | [symbol] |
| ATOM | 3794 | CA | LYS | A | 366 | 22.284 | 21.522 | 57.454 | 1.00 | 24.37 | [symbol] |
| ATOM | 3796 | CB | LYS | A | 366 | 20.933 | 21.079 | 56.920 | 1.00 | 24.52 | [symbol] |
| ATOM | 3799 | CG | LYS | A | 366 | 20.046 | 20.537 | 58.053 | 1.00 | 24.53 | [symbol] |
| ATOM | 3802 | CD | LYS | A | 366 | 19.425 | 19.211 | 57.731 | 1.00 | 24.45 | [symbol] |
| ATOM | 3805 | CE | LYS | A | 366 | 18.536 | 18.713 | 58.859 | 1.00 | 24.03 | [symbol] |
| ATOM | 3808 | NZ | LYS | A | 366 | 17.310 | 18.004 | 58.315 | 1.00 | 23.21 | [symbol] |
| ATOM | 3812 | C | LYS | A | 366 | 22.183 | 22.861 | 58.160 | 1.00 | 24.51 | [symbol] |
| ATOM | 3813 | O | LYS | A | 366 | 22.939 | 23.126 | 59.107 | 1.00 | 24.62 | [symbol] |
| ATOM | 3814 | O | SAH | E | 501 | 8.297 | −2.058 | 44.325 | 1.00 | 40.90 | [symbol] |
| ATOM | 3815 | C | SAH | E | 501 | 8.314 | −0.968 | 43.757 | 1.00 | 41.24 | [symbol] |
| ATOM | 3816 | CA | SAH | E | 501 | 9.219 | 0.183 | 44.185 | 1.00 | 41.19 | [symbol] |
| ATOM | 3818 | N | SAH | E | 501 | 9.794 | −0.121 | 45.501 | 1.00 | 41.18 | [symbol] |
| ATOM | 3819 | CB | SAH | E | 501 | 10.235 | 0.369 | 43.049 | 1.00 | 40.73 | [symbol] |
| ATOM | 3822 | CG | SAH | E | 501 | 10.669 | 1.774 | 42.642 | 1.00 | 40.21 | [symbol] |
| ATOM | 3825 | SD | SAH | E | 501 | 9.362 | 2.829 | 41.941 | 1.00 | 40.07 | [symbol] |
| ATOM | 3826 | C5* | SAH | E | 501 | 9.401 | 4.162 | 43.144 | 1.00 | 39.88 | [symbol] |
| ATOM | 3829 | C4* | SAH | E | 501 | 8.664 | 3.778 | 44.420 | 1.00 | 40.55 | [symbol] |
| ATOM | 3831 | O4* | SAH | E | 501 | 9.530 | 2.983 | 45.238 | 1.00 | 40.00 | [symbol] |
| ATOM | 3832 | C1* | SAH | E | 501 | 9.449 | 3.389 | 46.590 | 1.00 | 40.22 | [symbol] |
| ATOM | 3834 | C2* | SAH | E | 501 | 8.352 | 4.430 | 46.689 | 1.00 | 40.93 | [symbol] |
| ATOM | 3836 | O2* | SAH | E | 501 | 7.161 | 3.829 | 47.167 | 1.00 | 41.07 | [symbol] |
| ATOM | 3838 | C3* | SAH | E | 501 | 8.191 | 4.947 | 45.270 | 1.00 | 41.32 | [symbol] |
| ATOM | 3840 | O3* | SAH | E | 501 | 6.840 | 5.321 | 45.046 | 1.00 | 41.73 | [symbol] |
| ATOM | 3842 | N9 | SAH | E | 501 | 10.788 | 3.906 | 46.999 | 1.00 | 40.52 | [symbol] |
| ATOM | 3843 | C8 | SAH | E | 501 | 11.866 | 4.115 | 46.184 | 1.00 | 40.63 | [symbol] |
| ATOM | 3845 | N7 | SAH | E | 501 | 12.911 | 4.552 | 46.930 | 1.00 | 40.77 | [symbol] |
| ATOM | 3846 | C5 | SAH | E | 501 | 12.513 | 4.611 | 48.221 | 1.00 | 40.71 | [symbol] |
| ATOM | 3847 | C6 | SAH | E | 501 | 13.174 | 4.980 | 49.395 | 1.00 | 41.16 | [symbol] |
| ATOM | 3848 | N6 | SAH | E | 501 | 14.445 | 5.380 | 49.372 | 1.00 | 40.82 | [symbol] |
| ATOM | 3851 | C4 | SAH | E | 501 | 11.186 | 4.199 | 48.278 | 1.00 | 40.49 | [symbol] |
| ATOM | 3852 | N3 | SAH | E | 501 | 10.520 | 4.152 | 49.451 | 1.00 | 40.16 | [symbol] |
| ATOM | 3853 | C2 | SAH | E | 501 | 11.180 | 4.520 | 50.620 | 1.00 | 41.78 | [symbol] |
| ATOM | 3855 | N1 | SAH | E | 501 | 12.490 | 4.933 | 50.576 | 1.00 | 39.76 | [symbol] |
| ATOM | 3859 | N | ALA | K | 1 | 8.996 | 19.908 | 38.353 | 1.00 | 12.39 | [symbol] |
| ATOM | 3861 | CA | ALA | K | 1 | 8.059 | 18.939 | 37.729 | 1.00 | 12.32 | [symbol] |
| ATOM | 3863 | CB | ALA | K | 1 | 8.537 | 18.584 | 36.343 | 1.00 | 12.87 | [symbol] |
| ATOM | 3867 | C | ALA | K | 1 | 7.923 | 17.674 | 38.576 | 1.00 | 11.87 | [symbol] |
| ATOM | 3868 | O | ALA | K | 1 | 8.855 | 17.304 | 39.301 | 1.00 | 11.99 | [symbol] |
| ATOM | 3871 | N | ARG | K | 2 | 6.723 | 17.102 | 38.604 | 1.00 | 10.65 | [symbol] |
| ATOM | 3873 | CA | ARG | K | 2 | 6.470 | 15.840 | 39.291 | 1.00 | 10.19 | [symbol] |
| ATOM | 3875 | CB | ARG | K | 2 | 4.974 | 15.667 | 39.504 | 1.00 | 10.49 | [symbol] |
| ATOM | 3878 | CG | ARG | K | 2 | 4.415 | 16.668 | 40.510 | 1.00 | 11.49 | [symbol] |
| ATOM | 3881 | CD | ARG | K | 2 | 3.077 | 16.269 | 41.101 | 1.00 | 13.38 | [symbol] |
| ATOM | 3884 | NE | ARG | K | 2 | 2.519 | 17.280 | 41.994 | 1.00 | 14.59 | [symbol] |
| ATOM | 3886 | CZ | ARG | K | 2 | 1.339 | 17.177 | 42.589 | 1.00 | 16.21 | [symbol] |
| ATOM | 3887 | NH1 | ARG | K | 2 | 0.571 | 16.115 | 42.374 | 1.00 | 16.66 | [symbol] |
| ATOM | 3890 | NH2 | ARG | K | 2 | 0.918 | 18.135 | 43.406 | 1.00 | 17.20 | [symbol] |
| ATOM | 3893 | C | ARG | K | 2 | 7.094 | 14.579 | 38.700 | 1.00 | 9.34 | [symbol] |
| ATOM | 3894 | O | ARG | K | 2 | 6.421 | 13.555 | 38.530 | 1.00 | 8.70 | [symbol] |
| ATOM | 3895 | N | THR | K | 3 | 8.389 | 14.649 | 38.415 | 1.00 | 8.38 | [symbol] |
| ATOM | 3897 | CA | THR | K | 3 | 9.111 | 13.512 | 37.888 | 1.00 | 8.36 | [symbol] |
| ATOM | 3899 | CB | THR | K | 3 | 8.979 | 13.404 | 36.364 | 1.00 | 9.04 | [symbol] |
| ATOM | 3901 | OG1 | THR | K | 3 | 10.130 | 13.974 | 35.730 | 1.00 | 11.22 | [symbol] |
| ATOM | 3903 | CG2 | THR | K | 3 | 7.815 | 14.212 | 35.820 | 1.00 | 8.63 | [symbol] |
| ATOM | 3907 | C | THR | K | 3 | 10.587 | 13.644 | 38.268 | 1.00 | 7.71 | [symbol] |
| ATOM | 3908 | O | THR | K | 3 | 11.070 | 14.728 | 38.566 | 1.00 | 6.59 | [symbol] |
| ATOM | 3909 | CM | M1L | K | 4 | 11.009 | 5.204 | 39.930 | 1.00 | 2.73 | [symbol] |
| ATOM | 3910 | NZ | M1L | K | 4 | 11.719 | 6.209 | 39.159 | 1.00 | 2.32 | [symbol] |
| ATOM | 3911 | CE | M1L | K | 4 | 11.574 | 7.615 | 39.523 | 1.00 | 2.66 | [symbol] |
| ATOM | 3914 | CD | M1L | K | 4 | 12.364 | 8.512 | 38.589 | 1.00 | 2.45 | [symbol] |
| ATOM | 3917 | CG | M1L | K | 4 | 12.084 | 9.965 | 38.945 | 1.00 | 2.40 | [symbol] |
| ATOM | 3920 | CB | M1L | K | 4 | 12.904 | 10.873 | 38.053 | 1.00 | 2.54 | [symbol] |
| ATOM | 3923 | CA | M1L | K | 4 | 12.449 | 12.304 | 38.194 | 1.00 | 2.52 | [symbol] |
| ATOM | 3925 | N | M1L | K | 4 | 11.029 | 12.295 | 37.865 | 1.00 | 3.37 | [symbol] |
| ATOM | 3926 | C | M1L | K | 4 | 13.197 | 13.193 | 37.235 | 1.00 | 3.01 | [symbol] |
| ATOM | 3928 | O | M1L | K | 4 | 12.962 | 13.222 | 36.056 | 1.00 | 2.56 | [symbol] |
| ATOM | 3929 | N | GLN | K | 5 | 14.584 | 13.478 | 37.797 | 1.00 | 7.22 | [symbol] |
| ATOM | 3931 | CA | GLN | K | 5 | 15.450 | 14.271 | 36.884 | 1.00 | 8.17 | [symbol] |
| ATOM | 3933 | CB | GLN | K | 5 | 16.054 | 13.370 | 35.828 | 1.00 | 8.43 | [symbol] |
| ATOM | 3936 | CG | GLN | K | 5 | 16.970 | 12.351 | 36.383 | 1.00 | 8.88 | [symbol] |
| ATOM | 3939 | CD | GLN | K | 5 | 18.081 | 11.997 | 35.430 | 1.00 | 9.35 | [symbol] |
| ATOM | 3940 | OE1 | GLN | K | 5 | 17.869 | 11.885 | 34.227 | 1.00 | 8.97 | [symbol] |
| ATOM | 3941 | NE2 | GLN | K | 5 | 19.276 | 11.807 | 35.972 | 1.00 | 9.42 | [symbol] |
| ATOM | 3944 | C | GLN | K | 5 | 14.750 | 15.431 | 36.194 | 1.00 | 8.64 | [symbol] |
| ATOM | 3945 | O | GLN | K | 5 | 15.022 | 15.707 | 35.035 | 1.00 | 8.44 | [symbol] |
| ATOM | 3948 | N | THR | K | 6 | 13.745 | 15.998 | 36.835 | 1.00 | 9.60 | [symbol] |
| ATOM | 3950 | CA | THR | K | 6 | 13.138 | 17.229 | 36.381 | 1.00 | 10.73 | [symbol] |
| ATOM | 3952 | CB | THR | K | 6 | 12.040 | 17.631 | 37.363 | 1.00 | 10.90 | [symbol] |
| ATOM | 3954 | OG1 | THR | K | 6 | 11.316 | 18.746 | 36.833 | 1.00 | 12.28 | [symbol] |
| ATOM | 3956 | CG2 | THR | K | 6 | 12.600 | 18.116 | 38.676 | 1.00 | 11.76 | [symbol] |
| ATOM | 3960 | C | THR | K | 6 | 14.104 | 18.390 | 36.122 | 1.00 | 11.09 | [symbol] |
| ATOM | 3961 | O | THR | K | 6 | 14.998 | 18.679 | 36.930 | 1.00 | 10.66 | [symbol] |
| ATOM | 3962 | N | ALA | K | 7 | 13.884 | 19.030 | 34.973 | 1.00 | 11.81 | [symbol] |
| ATOM | 3964 | CA | ALA | K | 7 | 14.630 | 20.184 | 34.495 | 1.00 | 12.53 | [symbol] |
| ATOM | 3966 | CB | ALA | K | 7 | 14.059 | 20.649 | 33.157 | 1.00 | 12.47 | [symbol] |
| ATOM | 3970 | C | ALA | K | 7 | 14.556 | 21.319 | 35.484 | 1.00 | 13.22 | [symbol] |
| ATOM | 3971 | O | ALA | K | 7 | 13.501 | 21.601 | 36.053 | 1.00 | 13.11 | [symbol] |
| ATOM | 3972 | N | ARG | K | 8 | 15.686 | 21.985 | 35.664 | 1.00 | 14.21 | [symbol] |
| ATOM | 3974 | CA | ARG | K | 8 | 15.771 | 23.093 | 36.588 | 1.00 | 14.87 | [symbol] |
| ATOM | 3976 | CB | ARG | K | 8 | 17.199 | 23.225 | 37.085 | 1.00 | 15.34 | [symbol] |
| ATOM | 3979 | CG | ARG | K | 8 | 17.295 | 23.916 | 38.407 | 1.00 | 16.87 | [symbol] |
| ATOM | 3982 | CD | ARG | K | 8 | 18.715 | 24.179 | 38.833 | 1.00 | 18.48 | [symbol] |
| ATOM | 3985 | NE | ARG | K | 8 | 18.899 | 25.527 | 39.347 | 1.00 | 19.64 | [symbol] |
| ATOM | 3987 | CZ | ARG | K | 8 | 19.763 | 25.839 | 40.293 | 1.00 | 20.69 | [symbol] |
| ATOM | 3988 | NH1 | ARG | K | 8 | 20.502 | 24.888 | 40.845 | 1.00 | 21.86 | [symbol] |
| ATOM | 3991 | NH2 | ARG | K | 8 | 19.885 | 27.095 | 40.703 | 1.00 | 20.91 | [symbol] |
| ATOM | 3994 | C | ARG | K | 8 | 15.329 | 24.395 | 35.926 | 1.00 | 14.75 | [symbol] |
| ATOM | 3995 | O | ARG | K | 8 | 15.383 | 24.535 | 34.715 | 1.00 | 14.65 | [symbol] |
| ATOM | 3996 | N | LYS | K | 9 | 14.897 | 25.343 | 36.745 | 1.00 | 14.92 | [symbol] |
| ATOM | 3998 | CA | LYS | K | 9 | 14.466 | 26.644 | 36.275 | 1.00 | 14.95 | [symbol] |
| ATOM | 4000 | CB | LYS | K | 9 | 13.143 | 27.010 | 36.942 | 1.00 | 15.30 | [symbol] |
| ATOM | 4003 | CG | LYS | K | 9 | 12.515 | 28.277 | 36.408 | 1.00 | 15.99 | [symbol] |
| ATOM | 4006 | CD | LYS | K | 9 | 12.015 | 28.080 | 34.985 | 1.00 | 16.93 | [symbol] |
| ATOM | 4009 | CE | LYS | K | 9 | 11.418 | 29.347 | 34.407 | 1.00 | 17.56 | [symbol] |
| ATOM | 4012 | NZ | LYS | K | 9 | 10.839 | 29.172 | 33.040 | 1.00 | 18.06 | [symbol] |
| ATOM | 4016 | C | LYS | K | 9 | 15.529 | 27.676 | 36.635 | 1.00 | 14.60 | [symbol] |
| ATOM | 4017 | O | LYS | K | 9 | 16.025 | 27.681 | 37.755 | 1.00 | 14.59 | [symbol] |
| ATOM | 4018 | N | TYR | K | 10 | 15.874 | 28.546 | 35.689 | 1.00 | 14.19 | [symbol] |
| ATOM | 4020 | CA | TYR | K | 10 | 16.904 | 29.561 | 35.916 | 1.00 | 14.11 | [symbol] |
| ATOM | 4022 | CB | TYR | K | 10 | 18.127 | 29.273 | 35.050 | 1.00 | 13.87 | [symbol] |
| ATOM | 4025 | CG | TYR | K | 10 | 18.817 | 27.984 | 35.439 | 1.00 | 13.76 | [symbol] |
| ATOM | 4026 | CD1 | TYR | K | 10 | 18.440 | 26.775 | 34.878 | 1.00 | 13.67 | [symbol] |
| ATOM | 4028 | CE1 | TYR | K | 10 | 19.061 | 25.596 | 35.241 | 1.00 | 14.13 | [symbol] |
| ATOM | 4030 | CZ | TYR | K | 10 | 20.074 | 25.616 | 36.175 | 1.00 | 14.21 | [symbol] |
| ATOM | 4031 | OH | TYR | K | 10 | 20.694 | 24.442 | 36.532 | 1.00 | 15.41 | [symbol] |
| ATOM | 4033 | CE2 | TYR | K | 10 | 20.462 | 26.800 | 36.751 | 1.00 | 13.59 | [symbol] |
| ATOM | 4035 | CD2 | TYR | K | 10 | 19.836 | 27.976 | 36.386 | 1.00 | 13.60 | [symbol] |
| ATOM | 4037 | C | TYR | K | 10 | 16.349 | 30.958 | 35.649 | 1.00 | 14.14 | [symbol] |
| ATOM | 4038 | O | TYR | K | 10 | 15.294 | 31.125 | 35.050 | 1.00 | 14.51 | [symbol] |
| ATOM | 4039 | OXT | TYR | K | 10 | 16.895 | 31.990 | 36.032 | 1.00 | 14.07 | [symbol] |

1. A crystal comprising at least a catalytically active portion of a SET 7/9 histone methyltransferase.

2. A crystal according to claim 1, comprising at least residues 117-366, preferably residues 108-366, of SET 7/9.

3. A crystal according to claim 1, comprising SET 7/9 in complex with a lysine-containing substrate peptide or protein and/or a cofactor.

4. A crystal according to claim 3, comprising SET 7/9 in complex with a lysine-containing substrate peptide or protein and a cofactor, wherein the lysine of the substrate which interacts with the SET 7/9 active site is methylated and wherein cofactor is unmethylated at the site whence a methyl group is normally transferred to the substrate.

5. A crystal in accordance with claim 1, having the atomic co-ordinates set out in Annex 1 or having a structure in which the atomic co-ordinates vary by less than 0.2 Å in any direction from those set out in Annex 1.

6. A crystal in accordance with claim 1, comprising at least a catalytically active portion of SET 7/9 in complex with a lysine-containing substrate peptide or protein, wherein the complex comprises one or more of the following interactions:

| SET 7/9 | |
|---|---|
| Residue | Relative Substrate Residue |
| Trp 260 | −3 |
| His 252 | −2 |
| Asp 256 | −2 |
| Ser 268 | −1 |
| Thr 266 | Lys O |
| Ser 268 | Lys O |
| Ser 268 | +1 |
| Val 355 | +1 |
| Lys 317 | +2 |

7. A method of selecting or designing a ligand for SET 7/9, which method comprises use of at least part of the atomic co-ordinate data contained in Annex 1 or data derivable therefrom.

8. A method according to claim 7 comprising use of data relating to at least 50%, preferably 75% and most preferably 95% of the atoms detailed in Annex I, or data derivable therefrom, or data in which the atomic co-ordinates used vary by less than 0.2 Å (preferably less than 0.1 Å) in any direction from the co-ordinates given in Annex 1.

9. A method according to claim 7, which involves the use of a computer system and/or computer readable medium.

10. A method according to claim 7, which comprises at least use of the atomic co-ordinate data contained in Annex 1 in respect of some or all of the amino residues of the SET 7/9 domain selected from the group consisting of: Tyr 245, His 252, Asp 256, Asn 263, Gly 264, Thr 266, Leu 267, Ser 268, Gly 292, His 293, Ala 295, Tyr 305, Lys 317, Tyr 335, and Tyr 337.

11. A method according to claim 7, comprising the step of testing in vitro the selected or designed ligand for ability to bind to SET 7/9, typically by contacting the ligand with SET 7/9 and measuring the amount of ligand, if any, bound to the SET 7/9.

12. A method according to claim 7, comprising the step of testing in vitro the ability of the selected or designed ligand to modulate the methyltransferase activity of SET 7/9 HMT.

13. A method according to claim 7, comprising the step of forming a complex comprising the selected or designed ligand and SET 7/9.

14. A method according to claim 13, comprising the step of forming a crystal comprising the complex and subjecting the crystal to X-ray diffraction analysis to investigate the binding of the ligand to SET 7/9.

15. A method according to claim 14, wherein the structure of the ligand is modified in view of the results of the X-ray diffraction analysis in order to optimise the characteristics of its binding to SET 7/9.

16. A method according to claim 15, wherein the optimised ligand is retested in vitro for ability to bind to SET 7/9 and/or modulate methyltransferase activity of SET 7/9 HMT.

17. A computer system and/or a computer readable medium comprising positional data relating to, or derivable form, at least part of the atomic co-ordinate data contained in Annex 1.

18. A computer system and/or a computer readable medium according to claim 9, comprising at least atomic co-ordinate data from Annex 1 (or data derivable therefrom) in respect of some or all of the amino acid residues of SET 7/9 domain selected from the group consisting of:
Tyr 245, His 252, Hsp 256, Asn 263, Gly 264, Thr 266, Leu 267, Ser 268, Gly 292, His 293, Ala 295, Tyr 305, Lys 317, Tyr 335, and Tyr 337.

19. A method of forming a crystal comprising at least part of a SET 7/9 domain, the method comprising the steps of:
forming a complex comprising at least a catalytically active portion of a SET 7/9 domain and a lysine-containing substrate peptide or protein; and crystallising the complex.

20. A method according to claim 19, wherein the complex further comprises a cofactor.

21. A method according to claim 20, wherein the lysine-containing substrate peptide or protein comprises a target lysine residue which has a methylated side chain and wherein the cofactor is unmethylated.

22. A method of forming a crystal comprising at least part of a SET 7/9 domain, the method comprising the steps of: selecting or designing a ligand in accordance with claim 7; forming a complex comprising the ligand and at least a catalytically active portion of a SET 7/9 domain; and crystallising the complex.

23. A method according to claim 19, wherein the complex comprises at least residues 117-366 of SET 7/9.

24. A ligand for SET 7/9 selected or designed by the method of claim 7.

25. A ligand for SET 7/9 conforming to general formula I or II: 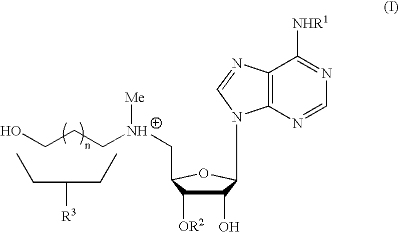
wherein R¹, R² and R³ are, independently: H, alkyl, haloakyl, aralkyl, acyl, cycloalkyl, alkenyl, or oxa-alkyl and wherein n=0, 1 or 2. 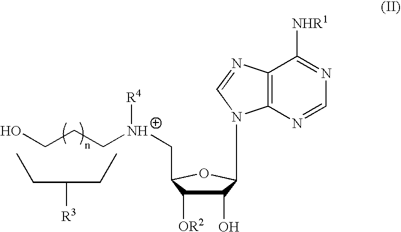
wherein R¹-R³ and are as in general formula I, and wherein R⁴ may be selected from any of the following: a linear, cyclic or branched alkyl group comprising 2 or more carbon atoms (preferably 2-6 carbon atoms, more preferably 2-5 carbon atoms); an alkenyl group having one or more double bonds; the alkyl or alkenyl group optionally being substituted at one or more positions (e.g. hydroxylated, halogenated, or aminated) and optionally comprising one or more oxa- aza- or thia-replacements of CH₂ groups.

26. A ligand for SET 7/9 comprising a bis-bicyclooctane or a 1,4 diphenyl moiety.

27. A ligand for SET 7/9 in accordance with the structure shown in FIG. 5 or FIG. 6.

28. A ligand according to claim 24, comprising an analogue of AdoMet having a stabilised 5′ thioadenosine moiety, and optionally substituted at the 3′-OH of the ribosyl moiety and/or the —NH₂ group at position 4 of the adenyl moiety.

29. A ligand for SET 7/9 according to claim 24 comprising a hydrophobic moiety which occupies the lysine access channel of SET 7/9, forming hydrophobic interactions with the amino acid residues of SET 7/9 which define the lysine access channel.

30. A ligand according to claim 24, comprising a moiety which alkylates the terminal amino group the target lysine side chain of the histone substrate.

31. A ligand according to claim 30, comprising a modified or unmodified 5′-aziridinyl adenosine alkylating agent.

32. A ligand according to claim 24, which modulates the methyltransferase activity of SET 7/9 HMT.

33. A pharmaceutical composition comprising a ligand according to claim 24, in admixture with a physiologically acceptable excipient, carrier or diluent.

34. Use of a ligand in accordance with claim 24 to prepare a pharmaceutical composition for modulating the methylation of histones.

35. A method of altering the substrate specificity of histone methyltransferases, the method comprising the steps of: obtaining a nucleotide sequence encoding at least a catalytically active portion of a SET HMT; and altering the nucleotide sequence so as to introduce a mutation at one or more residues selected from the group consisting of residue numbers; 245; 305; and 335.

36. A method according to claim 35 for altering the substrate specificity of a SET 7/9 HMT, the method comprising the step of introducing at least one of the mutations selected from the group consisting of Y245>A; Y305>; and Y305>P.

37. An SET HMT having altered substrate specificity as a result of alteration of the amino acid sequence of the protein by a method according to claim 35.
